SEQUENCE LISTING (19 sequences)

SEQ ID NO: 1 - DNA, 357 bp, Artificial Sequence
  source: /note=“Description of Artificial Sequence Synthetic polynucleotide”
gaggtgcagctgctggagagcggcggagggctggtccagccaggcggcagcctgaggctgtcctgcgccgccagcggcttcaccttcagccactacgtgatggcctgggtgcggcaggccccaggcaagggcctggaatgggtgtccagcatcagcagcagcggcggctggaccctgtacgccgacagcgtgaagggcaggttcaccatcagcagggacaacagcaagaacaccctgtacctgcagatgaacagcctgagggccgaggacaccgccgtgtactactgcaccaggggcctgaagatggccaccatcttcgactactggggccagggcaccctggtgaccgtgagcagc

SEQ ID NO: 2 - protein, 119 aa, Artificial Sequence [sequence omitted]

SEQ ID NO: 3 - DNA, 333 bp, Artificial Sequence
  source: /note=“Description of Artificial Sequence Synthetic polynucleotide”
cagtccgccctgacccagcccgccagcgtgagcggcagcccaggccagagcatcaccatcagctgcaccggcaccagcagcgacgtgggcagctacaacgtggtgtcctggtatcagcagcaccccggcaaggcccccaagctgatcatctacgaggtgtcccagaggcccagcggcgtgagcaacaggttcagcggcagcaagagcggcaacaccgccagcctgaccatcagcggcctgcagaccgaggacgaggccgactactactgctgcagctacgccggcagcagcatcttcgtgatcttcggcggagggaccaaggtgaccgtccta

SEQ ID NO: 4 - protein, 111 aa, Artificial Sequence [sequence omitted]

SEQ ID NO: 5 - protein, 5 aa, Artificial Sequence
  source: /note=“Description of Artificial Sequence Synthetic peptide”
HYVMA

SEQ ID NO: 6 - protein, 17 aa, Artificial Sequence
  source: /note=“Description of Artificial Sequence Synthetic peptide”
SISSSGGWTLYADSVKG

SEQ ID NO: 7 - protein, 10 aa, Artificial Sequence
  source: /note=“Description of Artificial Sequence Synthetic peptide”
GLKMATIFDY

SEQ ID NO: 8 - protein, 14 aa, Artificial Sequence
  source: /note=“Description of Artificial Sequence Synthetic peptide”
TGTSSDVGSYNVVS

SEQ ID NO: 9 - protein, 7 aa, Artificial Sequence
  source: /note=“Description of Artificial Sequence Synthetic peptide”
EVSQRPS

SEQ ID NO: 10 - protein, 11 aa, Artificial Sequence
  source: /note=“Description of Artificial Sequence Synthetic peptide”
CSYAGSSIFVI

SEQ ID NO: 11 - protein, 1321 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITQGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRSLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 12 - protein, 445 aa, Artificial Sequence
  source: /note=“Description of Artificial Sequence Synthetic polypeptide”
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 217 aa, Artificial Sequence
  source: /note=“Description of Artificial Sequence Synthetic polypeptide”
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 14 - DNA, 22 bp, Artificial Sequence
  source: /note=“Description of Artificial Sequence Synthetic primer”
atgcttgctggtgccgtggtca

SEQ ID NO: 15 - DNA, 25 bp, Artificial Sequence
  source: /note=“Description of Artificial Sequence Synthetic primer”
ggtctttcaccatgaagcactcccc

SEQ ID NO: 16 - DNA, 20 bp, Artificial Sequence
  source: /note=“Description of Artificial Sequence Synthetic primer”
agagctggatgcaccattgg

SEQ ID NO: 17 - DNA, 23 bp, Artificial Sequence
  source: /note=“Description of Artificial Sequence Synthetic primer”
tgaagactctgctgctgtttgtg

SEQ ID NO: 18 - DNA, 23 bp, Artificial Sequence
  source: /note=“Description of Artificial Sequence Synthetic primer”
gttttctccttctccgcacattt

SEQ ID NO: 19 - DNA, 23 bp, Artificial Sequence
  source: /note=“Description of Artificial Sequence Synthetic primer”
tatctcgaggggtttgaaaggtc

(19) United States
(12) Patent Application Publication
(10) Pub. No.: US 2022/0288204 A1
(43) Pub. Date: Sep. 15, 2022

(54) DOSAGE AND ADMINISTRATION OF ANTI-ERBB3 (HER3) MONOCLONAL ANTIBODIES TO TREAT TUMORS ASSOCIATED WITH NEUREGULIN 1 (NRG1) GENE FUSIONS

(71) Applicant: ELEVATION ONCOLOGY, INC., New York, NY (US)

(72) Inventors: Shawn M. LELAND, Aurora, CO (US); Lori Kunkel, San Francisco, CA (US); Doug Plessinger, Cincinnati, OH (US); Valerie Malyvanh Jansen, Nolensville, TN (US)

(21) Appl. No.: 17/691,645

(22) Filed: Mar. 10, 2022

Related U.S. Application Data
(60) Provisional application No. 63/159,575, filed on Mar. 11, 2021.

(51) Int. Cl. A61K 39/395; C07K 16/32; A61K 45/06; G01N 33/574; C12Q 1/6886

(52) U.S. Cl. CPC A61K 39/39558; C07K 16/32; A61K 45/06; G01N 33/57484; C12Q 1/6886; A61K 2039/545

(57) ABSTRACT

Provided are methods for the clinical treatment of tumors associated with NRG1 gene fusions using anti-ERBB3 antibodies.

### CROSS REFERENCE TO RELATED APPLICATION

This application claims the benefit of U.S. Provisional Application No. 63/159,575, filed Mar. 11, 2021, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Mar. 9, 2022, is named FNJ-026_Sequence_Listing.txt and is 24,462 bytes in size.

### BACKGROUND

Neuregulin-1 (NRG1) gene fusions represent an emerging, potentially actionable oncogenic driver across many different cancer types (Drilon A, et al. (2018) Cancer Discov; 8:686-959). NRG1 fusions have been detected in a variety of tumor types and include proteins that retain the extracellular EGF-like domain of NRG1 and the transmembrane domain of the specific fusion partner. These proteins serve as ligands for ERBB3 (HER3) and ERBB4 (HER4) receptors (Fernandez-Cuesta L, et al. (2014) Cancer Discov; 4:415-22). ERBB3 can then be activated through juxtacrine signaling from the EGF-like domain and autocrine signaling of secreted NRG1 (Wen D, et al. (1994) Mol Cell Biol 1994; 14:1909-199). Subsequent heterodimerization of ERBB3 with ERBB2 activates pathologic downstream signaling important in tumorigenesis which is mediated by pathways including ERK, PI3K, AKT, and NFκB.

NRG1 fusions are rare and recurring clinically actionable chromosomal translocations identified in 0.1-0.2% of all tumors (see e.g., Jonna C, et al. (2019) Clin. Cancer Res. 25:4966-4972; Drilon A, et al. (2018) Cancer Discov. 8:686-695). A fusion involving the neuregulin-1 gene (NRG1) was first identified in a breast cancer cell line in 1997 (Schaefer G, et al. (1997) Oncogene 15:1385-1394). Subsequently, fusions of the NRG1 gene with many different upstream partners have been shown to be expressed in lung and other cancers by several groups (Jonna C, et al. (2019) Clin. Cancer Res. 25:4966-4972; Drilon A, et al. (2018) Cancer Discov. 8:686-695). In the largest study that looked at the distribution of NRG1 fusions among different cancer types, 14/21,858 tumors had a fusion and the incidences varied widely by tumor type—0.5% gallbladder cancer, 0.5% renal clear cell carcinoma, 0.5% pancreatic cancer, 0.4% ovarian cancer and 0.2% sarcoma (Jonna C, et al. (2019) Clin. Cancer Res. 25:4966-4972). The incidence in non-small cell lung cancer and breast cancer is approximately 0.2% (Jonna C, et al. (2019) Clin. Cancer Res. 25:4966-4972). NRG1 fusions have also been identified in uterine cancer and head and neck cancer (Drilon A, et al. (2018) Cancer Discov. 8:686-695).

Despite improvements in tumor therapies, there are currently no approved therapies that specifically target NRG1 fusion for patients with advanced NRG1 fusion-positive solid tumors. Available chemotherapy, immunotherapy, and off-label therapies do not offer meaningful clinical benefit for this genomically defined patient population as exemplified in advanced NRG1 fusion-positive NSCLC (see, e.g., Drilon A, et al. (J. Clin. Oncol. 2021 Sep. 1; 39(25):2791-2802). Thus, there remains a critical need to optimize established therapies and develop new, promising therapies which prolong patients' lives while maintaining a high quality of life, particularly in the case of advanced cancers or metastatic solid tumors. Accordingly, it is an object of the present invention to provide methods that effectively inhibit ERBB3 signaling and can be used to treat and diagnose a variety of tumors associated with NRG1 fusions.

### SUMMARY

Provided herein are methods for treating a tumor in a human patient, wherein the tumor comprises an NRG1 fusion gene, by administering to the patient an anti-ERBB3 antibody according to a particular clinical dosage regimen (i.e., at a particular dose amount and according to a specific dosing schedule). The methods described herein are particularly advantageous in that they achieve a steady state concentration of the antibody and deliver maximal inhibition of ERBB3 pathway activity in patients harboring the NRG1 fusion gene, which is a known oncogenic driver associated with poor prognosis. In one embodiment, the human patient suffers from a tumor (e.g., a locally advanced or metastatic solid tumor).

Any suitable anti-ERBB3 antibody can be used in the methods described herein. An exemplary anti-ERBB3 antibody is seribantumab (also known as “FTN001” and “MM-121”). In one embodiment, the antibody comprises a heavy chain variable region (VH) encoded by the nucleic acid sequence set forth in SEQ ID NO:1. In another embodiment, the antibody comprises a light chain variable region (VL) encoded by the nucleic acid sequence set forth in SEQ ID NO:3. In another embodiment, the antibody comprises a VH and VL encoded by the nucleic acid sequences set forth in SEQ ID NOs:1 and 3, respectively. In another embodiment, the antibody comprises a VH comprising the amino acid sequence set forth in SEQ ID NO:2. In another embodiment, the antibody comprises a VL comprising the amino acid sequence set forth in SEQ ID NO:4. In another embodiment, the antibody comprises VH and VL regions comprising the amino acid sequences set forth in SEQ ID NOs: 2 and 4, respectively. In another embodiment, the antibody comprises (in amino-to carboxy-terminal order) CDRH1, CDRH2, and CDRH3 sequences comprising the amino acid sequences set forth in SEQ ID NO: 5 (CDRH1) SEQ ID NO: 6 (CDRH2) and SEQ ID NO: 7 (CDRH3), and/or (in amino-to carboxy-terminal order) CDRL1, CDRL2, and CDRL3 sequences comprising the amino acid sequences set forth in SEQ ID NO: 8 (CDRL1) SEQ ID NO: 9 (CDRL2) and SEQ ID NO: 10 (CDRL3). In another embodiment, the antibody comprises a heavy chain (HC) comprising the amino acid sequence set forth in SEQ ID NO:12. In another embodiment, the antibody comprises a light chain (LC) comprising the amino acid sequence set forth in SEQ ID NO:13. In another embodiment, the antibody comprises a HC and LC comprising the amino acid sequences set forth in SEQ ID Nos: 12 and 13, respectively. In another embodiment, an antibody is used that competes for binding with and/or binds to the same epitope on human ERBB3 as the above-mentioned antibodies. In a particular embodiment, the epitope comprises residues 92-104 of human ERBB3 (SEQ ID NO: 11). In another embodiment, the antibody competes with the antibody for binding to human ERBB3 and has at least 90% variable region amino acid sequence identity with the above-mentioned anti-ERBB3 antibodies (see, e.g., U.S. Pat. Nos. 7,846,440 and 8,691,225, the contents of which are expressly incorporated herein by reference). In another embodiments, the antibody comprises a biosimilar of seribantumab.

In another embodiment, methods for treating a subject (e.g., human patient) having a tumor that comprises an NRG1 fusion gene are provided, wherein the method comprises administering to the subject a therapeutically effective amount of an ERBB3 (HER3) antibody, wherein the antibody is administered at a dose of between about 2,000 mg to about 4,000 (e.g., at a dose of 2,000 mg, 2,250 mg, 2,500 mg, 2,750 mg, 3,000 mg, 3,250 mg, 3,500 mg, 3, 750 mg, or 4,000 mg) once weekly. In one embodiment, the antibody is administered intravenously at a once weekly dose of 3,000 mg unless disease progression or unacceptable toxicity

In another embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of between about 2,000 mg to about 4,000 (e.g., at a dose of 2,000 mg, 2,250 mg, 2,500 mg, 2,750 mg, 3,000 mg, 3,250 mg, 3,500 mg, 3, 750 mg, or 4,000 mg), and wherein the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively.

In another embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of 3,000 mg.

In another embodiment, methods for treating a subject having a tumor that comprises an NRG1 fusion gene are provided, wherein the method comprises administering to the subject a therapeutically effective amount of an 3ERBB3 (HER3) antibody, wherein the antibody is administered at a once weekly dose of 3,000 mg, and wherein the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively.

In another embodiment, methods for treating a subject having a tumor that comprises an NRG1 fusion gene are provided, wherein the method comprises administering to the subject a therapeutically effective amount of an 3ERBB3 (HER3) antibody, wherein the antibody is administered at a once weekly dose of 3,000 mg, and wherein the antibody comprises heavy and light chain variable region amino acid sequences comprising SEQ ID NOs: 2 and 4, respectively.

In another embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of 3,000 mg, and wherein the antibody comprises heavy chain and light chain amino acid sequences comprising SEQ ID NOs: 12 and 13, respectively.

In certain embodiments, the dosage regimen is adjusted to provide the optimum desired response (e.g., an effective response). For example, in some embodiments, administration of the antibody once weekly is discontinued if it is insufficient to effect treatment (e.g., as evidenced by clinical disease progression, increased symptoms, tolerance, and/or no clinical improvement compared to baseline). A determination that administration once weekly is insufficient to effect treatment can be made by any suitable means. In one embodiment, the determination is assessed by radiographic assessment (e.g., via computerized tomography (CT), positron emission tomography (PET) and/or magnetic resonance imaging (MRI)). In another embodiment, the determination is assessed by “Response Evaluation Criteria in Solid Tumors” (RECIST) version 1.1 guidelines (see, e.g., Eisenhauer, E. et al., (2009), “New response evaluation criteria in solid tumors: prevised RECIST guideline (version 1.1),” European Journal of Cancer (Oxford, England: 1990), 45(2), 228-47)). In another embodiment, the determination is assessed by liver function tests (LFT). In another embodiment, the determination is assessed by one or more disease (e.g., tumor) markers (e.g., carbohydrate antigen (CA19-9), cancer embryonic antigen (CEA), cancer antigen 125 (CA-125), and cancer antigen 15-3 (CA 15-3).

In another embodiment, the treatment is discontinued for up to three weeks if the subject experiences a clinically significant adverse event (e.g., Grade ≥3). An exemplary clinically significant adverse event includes, but is not limited to, hematologic toxicity (e.g., febrile neutropenia, neutropenic infection, Grade 4 neutropenia >7 days, Grade ≥3 thrombocytopenia for >7 days, Grade ≥3 thrombocytopenia with clinically significant bleeding, Grade 4 thrombocytopenia, and Grade ≥3 anemia >7 days). Another exemplary clinically significant adverse event is non-hematologic toxicity (e.g., (1) Grade ≥3 nausea, vomiting, or diarrhea lasting more than 72 hours despite optimal medical support with anti-emetics or anti-diarrheals, (2) Grade 4 (life-threatening) vomiting, or diarrhea, irrespective of duration, (3) any other grade ≥3 adverse event, except Grade ≥3 fatigue and anorexia lasting for <7 days or Grade ≤2 infusion related reactions).

In another embodiment, the once weekly antibody dose is reduced upon resuming treatment after the subject experience a clinically significant adverse event (e.g., Grade ≥3). For example, the once weekly antibody dose is reduced by 5%, 10%, 15%, 20%, 25%, or 30% upon resuming treatment after the subject experiences a clinically significant adverse event. In one embodiment, the once weekly antibody dose is reduced by 25% upon resuming treatment after the subject experiences a clinically significant adverse event. In another embodiment, the once weekly antibody dose is reduced to 2,750 mg, 2,500 mg, 2,250 mg, 2,000 mg, 1,750 mg, or 1,500 mg upon resuming treatment after the subject experiences a clinically significant adverse event. In one embodiment, the once weekly antibody dose is reduced to 2,250 mg upon resuming treatment after the subject experiences a clinically significant adverse event.

In another embodiment, the once weekly antibody dose is reduced by 50% upon resuming treatment after the subject experiences two or more clinically significant adverse events (e.g., Grade ≥3). For example, the once weekly antibody dose is reduced by 5%, 10%, 15%, 20%, 25%, 30%, 35%, 40%, 45%, 50%, 55%, 60%, 65%, or 70% upon resuming treatment after the subject experiences two or more clinically significant adverse events. In one embodiment, the once weekly antibody dose is reduced by 50% upon resuming treatment after the subject experiences two or more clinically significant adverse events. In another embodiment, the once weekly antibody dose is reduced to 2,250 mg, 2,000 mg, 1,750 mg, 1,500 mg, 1,250 mg, 1,000 mg, 750 mg, or 500 mg upon resuming treatment after the subject experiences two or more clinically significant adverse events. In one embodiment, the once weekly antibody dose is reduced to 1,500 mg upon resuming treatment after the subject experiences two or more clinically significant adverse events.

In one embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of 3,000 mg, and wherein the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively, and

wherein the once weekly antibody dose is reduced by 25% or more (e.g., reduced to 2,750 mg, 2,500 mg, 2,250 mg, 2,000 mg, 1,750 mg, or 1,500 mg) upon resuming treatment after the subject experiences a clinically significant adverse event.

In one embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of 3,000 mg, wherein the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively, and wherein the once weekly antibody dose is reduced by 50% or more (e.g., reduced to 2,250 mg, 2,000 mg, 1,750 mg, 1,500 mg, 1,250 mg, 1,000 mg, 750 mg, or 500 mg) upon resuming treatment after the subject experiences two or more clinically significant adverse events.

In another aspect, methods for treating a subject (e.g., human patient) having a tumor that comprises an NRG1 fusion gene are provided, wherein the method comprises administering to the subject a therapeutically effective amount of an ERBB3 (HER3) antibody, wherein the antibody is administered at a once weekly dose of between about 2,000 mg to about 4,000 (e.g., at a dose of 2,000 mg, 2,250 mg, 2,500 mg, 2,750 mg, 3,000 mg, 3,250 mg, 3,500 mg, 3,750 mg, or 4,000 mg). For example, in one embodiment, the antibody is administered at a dose of 2,000 mg once a week. In another embodiment, the antibody is administered at a dose of 2,250 mg once a week. In another embodiment, the antibody is administered at a dose of 2,500 mg once a week. In another embodiment, the antibody is administered at a dose of 2,750 mg once a week. In another embodiment, the antibody is administered at a dose of 3,000 mg once a week. In another embodiment, the antibody is administered at a dose of 3,250 mg once a week. In another embodiment, the antibody is administered at a dose of 3,550 mg once a week. In another embodiment, the antibody is administered at a dose of 3,750 mg once a week. In another embodiment, the antibody is administered at a dose of 4,000 mg once a week. In another embodiment, the antibody is administered at a once weekly dose of between about 2,000 mg to about 4,000 (e.g., at a dose of 2,000 mg, 2,250 mg, 2,500 mg, 2,750 mg, 3,000 mg, 3,250 mg, 3,500 mg, 3,750 mg, or 4,000 mg) until intolerance (e.g., unmanageable toxicity). In another embodiment, the antibody is administered at a once weekly dose of between about 2,000 mg to about 4,000 (e.g., at a dose of 2,000 mg, 2,250 mg, 2,500 mg, 2,750 mg, 3,000 mg, 3,250 mg, 3,500 mg, 3,750 mg, or 4,000 mg) until progressive disease (PD). In one embodiment, the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively. In another embodiment, the antibody comprises VH and VL regions comprising the amino acid sequences set forth in SEQ ID NOs: 2 and 4, respectively. In another embodiment, the antibody comprises a HC and LC comprising the amino acid sequences set forth in SEQ ID Nos: 12 and 13, respectively.

The anti-ERBB3 antibody can be administered to a subject by any suitable means. For example, in one embodiment, the antibody is administered intravenously. In another embodiment, the antibody is administered intravenously over about one hour.

The treatment methods described herein can be continued for as long as clinical benefit is observed or until unmanageable toxicity or disease progression occurs. For example, in one embodiment, the treatment is continued for 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, or three years or more.

The efficacy of the treatment methods provided herein can be assessed using any suitable means. In one embodiment, the treatment produces at least one therapeutic effect selected from the group consisting of reduction in size of a tumor, reduction in number of metastatic lesions over time, complete response, partial response, and stable disease.

In another embodiment, the subject has been determined to have a tumor comprising an NRG1 fusion gene, e.g., as measured by a tumor biopsy or liquid biopsy assay. In another embodiment, the assay includes the polymerase chain reaction (PCR), fluorescence in situ hybridization (FISH), or next-generation sequencing (NGS), e.g., an RNA-based or DNA-based testing.

In another embodiment, the subject has locally advanced or metastatic solid tumor. In another embodiment, the subject has an advanced refractory solid tumor. Non-limiting examples of cancers for treatment include squamous cell carcinoma, lung cancer (e.g., invasive mucinous adenocarcinoma (IMA), small-cell lung cancer, non-small cell lung cancer, squamous non-small cell lung cancer (NSCLC), non-squamous NSCLC), glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, pancreatic ductal adenocarcinoma (PDAC), glioblastoma (glioblastoma multiforme), cervical cancer, stomach cancer, bladder cancer, gallbladder cancer (GBC), hepatoma, breast cancer, colon carcinoma, and head and neck cancer (or carcinoma), diffuse large B-cell lymphoma (DLBCL), neuroendocrine tumor of the nasopharynx, gastric cancer, germ cell tumor, sarcoma, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, gastroesophageal junction (GEJ) cancer, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)). In one embodiment, the subject has IMA. In another embodiment, the subject has ovarian cancer.

In another embodiment, the NRG1 fusion comprises a gene selected from the group consisting of, but not limited to: DOC4, CLU, STMN2, PCM1, cluster of differentiation 74 (CD74); solute carrier family 3 member 2 (SLC3A2); syndecan-4 (SDC4); ATPase subunit beta-1 (ATP1B1); rho-associated, coiled-coil-containing protein kinase 1 (ROCK1); forkhead box protein A1 (FOXA1); A-kinase anchor protein 13 (AKAP13); thrombospondin 1 (THBS1); high affinity cAMP-specific 3′,5′-cyclic phosphodiesterase 7A (PDE7A); THAP domain-containing protein 7 (THAP7); SMAD4; RAB3A interacting protein like 1 (RAB3IL1); prostate transmembrane protein, androgen induced 1 (PMEPA1); stathmin 2 (STMN2); solute carrier family 3 member 2 (SLC3A2); vesicle associated membrane protein 2 (VAMP2); RNA-binding protein with multiple splicing (RBPMS); integrator complex subunit 9 (INTS9); WRN RecQ like helicase (WRN); RAB2A interacting protein like 1 (RAB2IL1); store-operated calcium entry associated regulatory factor) (SARAF); amyloid precursor protein (APP); kinesin family member 13B (KIF13B); ADAM metallopeptidase domain 9 (ADAMS); cadherin 1 (CDH1); COX10 antisense RNA 1 (COX10-AS1); disco interacting protein 2 homolog B (DIP2B); dihydropyrimidinase-related protein 2 (DPYSL2); growth/differentiation factor 15 (GDF15); homeobox containing 1 (HMBOX1); midkine (MDK); mitochondrial ribosomal protein L13 (MRPL13); notch receptor 2 (NOTCH2); poly(ADP-ribose) polymerase family member 8 (PARP8); protein O-mannose kinase (POMK); SET domain-containing protein 4 (SETD4); tenascin C (TNC); teashirt zinc finger homeobox 2 (TSHZ2); V-set domain-containing T-cell activation inhibitor 1 (VTCN1); Wolf-Hirschhorn syndrome candidate-1 (WHSC1L1); and zinc finger MYM-type protein 2 (ZMYM2).

In another embodiment, the anti-ERBB3 antibody is administered with a second targeted therapeutic agent, such as a small molecule inhibitor or an antibody, e.g., against ERBB2 (HER2), ERBB3, ERBB4, epidermal growth factor receptor (EGFR), insulin like growth factor 1 receptor (IGF1-R), tyrosine-protein kinase Met (C-MET), Lewis Y, mucin 1 (MUC-1), epithelial cell adhesion molecule (EpCAM), cancer antigen 125 (CA125), prostate specific membrane antigen (PSMA), platelet-derived growth factor receptor alpha (PDGFR-α), platelet-derived growth factor receptor beta (PDGFR-β), proteo-oncogene c-kit (C-KIT), or an fibroblast growth factor (FGF) receptor. In yet another embodiment, the anti-ERBB3 antibody and the second therapeutic agent are administered simultaneously (e.g., in a single formulation or concurrently as separate formulations). Alternatively, in another embodiment, the anti-ERBB3 antibody and the second therapeutic agent are administered sequentially (e.g., as separate formulations). In yet another embodiment, the anti-ERBB3 antibody is linked to a second therapeutic agent, e.g., an ERBB inhibitor. For example, the second therapeutic agent is a targeted therapeutic, such as a small molecule inhibitor or an antibody, e.g., against ERBB2 (HER2), ERBB3, ERBB4, EGFR, IGF1-R, C-MET, Lewis Y, MUC-1, EpCAM, CA125, prostate specific membrane antigen (PSMA), PDGFR-α, PDGFR-β, C-KIT, or an FGF receptor, which is linked to the anti-ERBB3 antibody.

In another embodiment, the methods described herein can be utilized in combination (e.g., simultaneously or separately) with another treatment, e.g., radiation, surgery, chemotherapy, immunotherapy (e.g., monoclonal antibodies and tumor-agnostic treatments (such as checkpoint inhibitors), oncolytic virus therapy, T-cell therapy, and/or cancer vaccines) or chemoimmunotherapy (e.g., one or more drugs to kill or slow the growth of cancer cells combined with treatments to stimulate or restore the ability of the immune system to fight cancer).

In another embodiment, the methods described herein further comprise inhibition (antagonism) of MET signaling pathway activity. Accordingly, in one embodiment, the methods described herein further comprise administration of a MET inhibitor. Exemplary MET inhibitors include, but are not limited to: Crizotinib, PHA-665752, SU11274, SGX-523, BMS-777607, JNJ-38877605, Tivantinib, PF-04217903, MGCD-265, Capmatinib, AMG 208, MK-2461, AMG 458, NVP-BVU972, and Tepotinib.

In another embodiment, the methods described herein further comprise inhibition (antagonism) of mTOR (mammalian target of rapamycin) signaling pathway activity. Accordingly, in one embodiment, the methods described herein further comprise administration of an mTOR inhibitor. In one embodiment, the mTOR inhibitor inhibits mTORC1. In another embodiment, the mTOR inhibitor inhibits mTORC2. In yet another embodiment, the mTOR inhibitor inhibits both mTORC1 and mTORC2. Exemplary mTOR inhibitors include, but are not limited to: gedatolisib, sirolimus, everolimus, temsirolimus, dactolisib, AZD8055, ABTL-0812, PQR620, GNE-493, KU0063794, torkinib, ridaforolimus, sapanisertib, voxtalisib, torin 1, torin 2, OSI-027, PF-04691502, apitolisib, GSK1059615, WYE-354, vistusertib, WYE-125132, BGT226, palomid 529, WYE-687, WAY600, GDC-0349, XL388, bimiralisib (PQR309), omipalisib (GSK2126458, GSK458), onatasertib (CC-223), samotolisib, omipalisib, RMC-5552, and GNE-477.

In another embodiment, the methods described herein further comprise administration of a RET inhibitor. In another embodiment, the methods described herein further comprise administration of a KRAS G12C inhibitor. In another embodiment, the methods described herein further comprise administration of an NTRK inhibitor. In another embodiment, the methods described herein further comprise administration of an EGFR inhibitor. In another embodiment, the methods described herein further comprise administration of an ALK inhibitor. In another embodiment, the methods described herein further comprise administration of a MEK inhibitor. In another embodiment, the methods described herein further comprise administration of an ERK inhibitor. In another embodiment, the methods described herein further comprise administration of an AKT inhibitor. In another embodiment, the methods described herein further comprise administration of a PI3K inhibitor.

In another embodiment, the methods described herein further comprise administration of one or more anti-estrogens, including, but not limited to, fulvestrant, an aromatase inhibitor, tamoxifen, a non-steroidal aromatase inhibitor (letrozole, anastrozole), a steroidal aromatase inhibitor (exemestane), novel selective estrogen receptor degraders (SERDs), and selective estrogen receptor modulators (SERMs)).

In yet another aspect, kits for treating a tumor comprising an NRG1 fusion gene in a subject are provided, wherein the kits comprise: a dose of an anti-ERBB3 antibody (e.g., FTN001) comprising CDRH1, CDRH2, and CDRH3 sequences comprising the amino acid sequences set forth, respectively, in SEQ ID NO: 5 (CDRH1) SEQ ID NO: 6 (CDRH2) and SEQ ID NO: 7 (CDRH3), and CDRL1, CDRL2, and CDRL3 sequences comprising the amino acid sequences set forth, respectively, in SEQ ID NO: 8 (CDRL1) SEQ ID NO: 9 (CDRL2) and SEQ ID NO: 10 (CDRL3), and instructions for using the anti-ERBB3 antibody in any of the methods described herein. In one embodiment, a kit of the invention comprises at least 3,000 mg of the anti-ERBB3 antibody.

### BRIEF DESCRIPTION OF THE DRAWINGS

FIGS. 1A-1H demonstrate that seribantumab inhibits growth of cells harboring NRG1 alterations. Expression of DOC4-NRG1 (FIG. 1A) or SLC3A2-NRG1 (FIG. 2B) fusions in MDA-MB-175-VII and LUAD-0061AS3 cells, respectively, was determined by RT-PCR. The HBECp53 (NRG1 fusion negative) and HBECp53-SLC3A2-NRG1 cells were used as negative and positive controls, respectively. Cells were treated with the indicated concentrations of seribantumab or afatinib for 96 hours and then the relative number of cells was estimated with AlamarBlue viability dye (FIG. 1C and FIG. 1D). Viability results represent the mean±SEM of 2-5 independent experiments in which each condition was assayed in triplicate determinations. Viability data were analyzed by nonlinear regression and the IC50 values for growth inhibition and the 95% confidence interval were determined with GraphPad Prism 8. Cells were treated as indicated with afatinib or seribantumab, and then counted every 24 to 48 hours (FIG. 1E, FIG. 1F, FIG. 1G, and FIG. 1H). Results represent the mean±SD for one experiment in which each condition was assayed in duplicate.

FIGS. 2A-2E show that seribantumab specifically blocks NRG1-dependent growth and induces apoptosis. MCF-7 cells were treated with the indicated concentrations of NRG1-b1 for 10 minutes, and then cell extracts were prepared and subjected to Western blotting for the indicated phosphorylated (p) or total (t) proteins (FIG. 2A). MCF-7 cells were treated with escalating doses of NRG1-b1 and seribantumab for 96 hours, and then growth was determined using AlamarBlue viability dye (FIG. 2B). Data were analyzed by nonlinear regression using GraphPad Prism 8. There were four replicates of each condition and data represent the mean±SD. C, MCF-7 cells were pretreated with 2 mmol/L seribantumab for 1 hour prior to stimulation with 10 ng/mL NRG1-b1. Cells were counted on the days indicated on the graph. Results represent the mean±SD of duplicates of each condition in one representative experiment. MDA-MB-175-VII (FIG. 2D) and LUAD-0061AS3 (FIG. 2E) cells were treated with the indicated concentrations of inhibitors for 48 hours, and then caspase 3/7 enzymatic activity was measured in cell homogenates. Carfilzomib (20S proteasome inhibitor) was used as a positive control for apoptosis. Results are the mean±SEM of three independent experiments in which each condition was assayed in three replicate determinations. The absence of an error bar for any data point indicates an error value too small to be represented on the scale used.

FIGS. 3A-3B show that seribantumab inhibits intracellular signaling in lung cancer cell lines with NRG1 fusion. Serum-depleted LUAD-0061AS3 (FIG. 3A) and HBECp53-CD74-NRG1 (FIG. 3B) cells were treated with the indicated concentrations of seribantumab or afatinib for 1 hour. Whole-cell extracts were prepared after all treatments and subjected to SDS-PAGE, followed by immunoblotting for the phosphorylated (p) or total (t) proteins shown in each panel. All Western blotting studies were conducted at least two times and representative immunoblots of phosphorylated (p) and total (t) proteins are shown.

FIGS. 4A-4D show that seribantumab inhibits intracellular signaling in breast cancer cells with NRG1 fusion. Serum-depleted MDA-MB-175-VII cells were treated with the indicated concentrations of seribantumab for 3 hours (FIG. 4A). Serum-depleted MDA-MB-175-VII cells were treated with 2 mmol/L seribantumab for up to 24 hours (FIG. 4B, FIG. 4C, and FIG. 4D). Whole-cell extracts were prepared after all treatments and subjected to SDS-PAGE, followed by immunoblotting for the phosphorylated (p) or total (t) proteins shown in each panel. All Western blotting studies were conducted at least two times and representative immunoblots of phosphorylated (p) and total (t) proteins are shown.

FIGS. 5A-5C demonstrate the efficacy of seribantumab in a NSCLC PDX model with NRG1 fusion. Characterization of the LUAD-0063AS1 PDX model. H&E staining, TTF-1, and phospho-HER3 IHC (FIG. 5A, left to right). Mice bearing LUAD-0061AS3 PDX tumors (seven animals/group) were treated with the indicated doses of afatinib [once daily (QD)] or seribantumab [twice weekly (BIW)] (FIG. 5B). Tumor volumes were measured twice weekly and plotted overtime. Results represent the mean±SEM. A zoom-in on the last 14 days of treatment (shaded area) to show that 1 mg seribantumab is as effective as the highest dose of afatinib (right). Mice bearing LUAD-0061AS3 PDX tumors were treated with a single administration of vehicle, afatinib, or seribantumab, and then tumors were collected at 2, 24, or 168 hours. Western blotting was performed twice for each protein and representative immunoblots of phosphorylated (p) and total (t) proteins are shown (FIG. 5C).

FIGS. 6A-6C show the efficacy of seribantumab in an ovarian cancer PDX model with NRG1 fusion. IHC characterization of the OV-10-0050 PDX model (FIG. 6A). H&E staining, WT1, and TP53 IHC (left to right). Mice bearing OV-10-0050 PDX tumors (5-8 animals/group) were treated with vehicle, afatinib [5 mg/kg, once daily (QD)], or seribantumab [twice weekly (BIW)] (FIG. 6B). Treatment was terminated on day 27 and animals were monitored for tumor regrowth until tumors reached maximum allowable size or until 90 days after treatment initiation. Results represent the mean tumor volume ±SEM. A zoom-in view on tumor volumes during the last 40 days of monitoring of seribantumab-treated groups (right). The highest dose of seribantumab blocked tumor regrowth after cessation of treatment. Change in the volume of individual tumors (day 27 vs. volume at start of treatment) (FIG. 6C).

FIGS. 7A-7E demonstrate that seribantumab inhibits phospho-HER3 and phospho-AKT activated by overexpression of NRG1 fusions in immortalized H6C7 human pancreatic ductal epithelial cells. H6C7-EV (empty vector) and H6C7-ATP1B1-NRG1 cells were profiled for activated intracellular kinases using phospho-proteomic arrays (FIG. 7A). H6C7-EV and H6C7 cells with the indicated NRG1 fusions were profiled for activated receptor tyrosine kinases (RTK) using phospho-RTK arrays (FIG. 7B). Quantitation of phosphorylated EGFR, HER2, and HER3 is shown (FIG. 7C). Western blot analysis of cell extracts from H6C7-EV and H6C7 cells expressing NRG1 fusions (FIGS. 7D-E). Cells were treated with the indicated concentrations of seribantumab and then whole-cell lysates profiled for the indicated phospho- and total proteins by Western blotting. All cells were serum-started for 24 hours prior to experimentation.

FIGS. 8A-8D demonstrate that seribantumab inhibits growth of NRG1-rearranged pancreatic adenocarcinoma PDX model (CTG-0943, APP-NRG1). Mice bearing CTG-0943 PDX tumors (5-8 mice per group) were treated with indicated agents (FIG. 8A). Representative H&E-stained slides of a vehicle-treated tumor (FIG. 8B). Tumor volume, results represent the mean±SEM. Animals in the seribantumab 5 mg and 10 mg groups were administered seribantumab 5 mg/kg and 10 mg/kg, respectively, for the first two doses (FIG. 8C). Change in the volume of individual tumors (vehicle: day 20; treated groups: day 31) compared with day 0(%) (FIG. 8D). Western blot analysis of vehicle, and afatinib and seribantumab-treated tumors. Tumor residues extracted day 24 for vehicle-, day 31 for seribantumab-, and day 32 for afatinib-treated groups. Antibody reactivity with human (H) and/or mouse (M) proteins is indicated.

FIGS. 9A-9E demonstrate that targeted combinations inhibit growth of NRG1-rearranged cholangiocarcinoma PDX model harboring additional known driver alterations (CH-17-0068, RBPMS-NRG1). Representative H&E-stained CH-17-0068 PDX tumor (FIG. 9A). Genomic alterations identified by RNAseq and corresponding investigational targeted therapies (FIG. 9B). Mice bearing CH-17-0068 PDX tumors (5-6 mice per group) were treated with seribantumab or afatinib monotherapy for 30 days (FIG. 9C). Afatinib (5 mg/kg QD) or AG-120 (isocitrate dehydrogenase [IDH] inhibitor, 150 mg/kg twice daily [BID]) were then added to the indicated groups. Results represent the mean±SEM. Change in the volume of individual tumors on day 30 (FIG. 9D) or at the end of the study (FIG. 9E).

FIGS. 10A-10B are representative computed tomography (CT) images of a liver metastasis in a patient with KRAS WT pancreatic cancer that harbors an ATP1B1-NRG1 gene fusion. The patient was treated with seribantumab. Liver metastasis prior to starting seribantumab from November 2020 (NTL1 liver seg 8 26×26 mm; FIG. 10A) and the same liver metastasis in June 2021 while the patient received seribantumab (NTL1 liver seg 8 resolved; FIG. 10B).

FIG. 11 is a plot of CA19-9 tumor marker and sum of target lesions in a patient with KRAS WT pancreatic cancer that harbors an ATP1B1-NRG1 gene fusion treated with seribantumab.

### DETAILED DESCRIPTION

### I. Definitions

As used herein, the term “subject” or “patient” is a human having a tumor that comprises an NRG1 fusion gene, e.g., a human determined to have a tumor (such as a locally advanced or metastatic solid tumor) which comprises an NRG1 fusion gene.

The term “neuregulin 1” or “NRG1” (also referred to as glial growth factor (GGF), HGL, HRG, NDF; acetylcholine receptor inducing activity (ARIA), GGF2, HRG1, HRGA, SMDF, MST131, MSTP131, or NRG1-IT2) is a membrane glycoprotein and one of four proteins in the neuregulin family that act on the EGFR family of receptors. The term “NRG1” includes variants, isoforms, homologs, orthologs and paralogs. NRG1 mediates cell-cell signaling and plays a critical role in the growth and development of multiple organ systems. A variety of different isoforms are produced from the NRG1 gene through alternative promoter usage and splicing. These isoforms are expressed in a tissue-specific manner and differ significantly in their structure, and are classified as types I, II, III, IV, V and VI. (Mei and Xiong (2008) Nat Rev Neurosci 9(6): 437-452). Dysregulation of this gene has been linked to diseases such as cancer, schizophrenia, cardiac disease, and bipolar disorder (BPD).

The term “fusion gene” refers to a hybrid gene comprising two previously separate genes, i.e., the two separate genes have become fused together. Fusion genes can occur as a result of translocation, interstitial deletion, or chromosomal inversion. Fusion genes are known to contribute to tumor formation by producing (i.e., expressing) the proteins encoded by the genes to form a fusion protein.

The term “NRG1 fusion gene” refers to a fusion gene comprising a gene encoding NRG1 (i.e., neuregulin 1), or a portion thereof, and a second gene encoding a second protein, or a portion thereof (i.e., a fusion partner). Expression of the two genes results in the formation of an NRG1 fusion protein (also referred to herein as “NRG1 fusion”). For example, an NRG1 fusion can include the extracellular EGF-like domain of NRG1 and the transmembrane domain of the fusion partner. In another embodiment, an NRG1 fusion gene lacks the EGF-like domain of NRG1. These proteins then serve as ligands for ERBB3 (HER3) and ERBB4 (HER4) receptors. ERBB3 can then be activated through juxtacrine signaling from the EGF-like domain and autocrine signaling of secreted NRG1. Subsequent heterodimerization of ERBB3 with ERBB2 activates downstream signaling important in tumorigenesis mediated by pathways including ERK, PI3K, AKT, and NFκB, described in cell models.

As used herein, the term “ERBB3” refers to the ERBB3 receptor which is a 148 kD transmembrane receptor belonging to the ErbB/EGFR receptor tyrosine kinase family although lacks intrinsic kinase activity. The ErbB receptors form homo- and heterodimeric complexes that impact the physiology of cells and organs by mediating ligand-dependent (and in some cases ligand independent) activation of multiple signal transduction pathways. ERBB3-containing heterodimers (such as ERBB2/ERBB3) in tumor cells have been shown to be the most mitogenic and oncogenic receptor complex within the ErbB family. Upon binding to its physiological ligand, the ERBB3 receptor dimerizes with other ErbB family members, predominantly ERBB2. ERBB3/ERBB2 dimerization results in transphosphorylation of ERBB3 on tyrosine residues contained within the cytoplasmic tail of the protein. Phosphorylation of these sites creates SH2 docking sites for SH2-containing proteins, including PI3-kinase. ERBB3-containing heterodimeric complexes are therefore potent activators of AKT, as ERBB3 possesses six tyrosine phosphorylation sites with YXXM motifs that, when phosphorylated, serve as excellent binding sites for phosphoinositol-3-kinase (PI3K), the action of which results in subsequent downstream activation of the AKT pathway. These six PI3K sites serve as a strong amplifier of ERBB3 signaling. Activation of this pathway further elicits several important biological processes involved in tumorigenesis, such as cell growth, migration and survival.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a tumor having an NRG1 fusion gene. Effective treatment may refer to alleviation of at least one symptom of cancer associated with the tumor. Such effective treatment may, e.g., reduce patient pain, reduce the size and/or number of lesions, may reduce or prevent metastasis of a tumor, and/or may slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to cancers, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay tumor development. In some embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of the anti-ERBB3 antibody clinically proven to result in a significant decrease in the growth of the tumor and/or slow progression of the cancer.

As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore, not provided as a mg/kg dose, but rather as an absolute amount of the agent (e.g., the anti-ERBB3 antibody).

The term “antibody” describes polypeptides comprising at least one antibody derived antigen binding site (e.g., VH/VL region or Fv, or complementarity determining region—CDR) that specifically binds to ERBB3. Accordingly, the term “antibody” as used to herein includes whole antibodies and any antigen binding fragments (i.e., “antigen-binding portions”) or single chains thereof. Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may to include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

### II. Anti-ERBB3 Antibodies

Any suitable anti-ERBB3 antibody can be used in the methods described herein. An exemplary anti-ERBB3 antibody suitable for use in the invention is seribantumab (also referred to as FTN001, MM-121, and “Ab #6” in U.S. Pat. No. 7,846,440), as well as functionally and/or structurally equivalent antibodies, i.e., variants of seribantumab which have the same activity as seribantumab. Antibodies for use in the invention can be generated using methods well known in the art.

In one embodiment, the antibody comprises a heavy chain variable region (VH) encoded by the nucleic acid sequence set forth in SEQ ID NO:1. In another embodiment, the antibody comprises a light chain variable region (VL) encoded by the nucleic acid sequence set forth in SEQ ID NO:3. In another embodiment, the antibody comprises a VH and VL encoded by the nucleic acid sequences set forth in SEQ ID NOs:1 and 3, respectively. In another embodiment, the antibody comprises a VH comprising the amino acid sequence set forth in SEQ ID NO:2. In another embodiment, the antibody comprises a VL comprising the amino acid sequence set forth in SEQ ID NO:4. In another embodiment, the antibody comprises VH and VL regions comprising the amino acid sequences set forth in SEQ ID NOs: 2 and 4, respectively. In another embodiment, the antibody comprises (in amino-to carboxy-terminal order) CDRH1, CDRH2, and CDRH3 sequences comprising the amino acid sequences set forth in SEQ ID NO: 5 (CDRH1) SEQ ID NO: 6 (CDRH2) and SEQ ID NO: 7 (CDRH3), and/or (in amino-to carboxy-terminal order) CDRL1, CDRL2, and CDRL3 sequences comprising the amino acid sequences set forth in SEQ ID NO: 8 (CDRL1) SEQ ID NO: 9 (CDRL2) and SEQ ID NO: 10 (CDRL3). In another embodiment, the antibody comprises a heavy chain (HC) comprising the amino acid sequence set forth in SEQ ID NO:12. In another embodiment, the antibody comprises a light chain (LC) comprising the amino acid sequence set forth in SEQ ID NO:13. In another embodiment, the antibody comprises a HC and LC comprising the amino acid sequences set forth in SEQ ID Nos: 12 and 13, respectively. In another embodiments, the antibody comprises a biosimilar of seribantumab. As used herein, a biosimilar is a product which is highly similar (e.g., in structure, function and property) to another already approved biological medicine (e.g., a reference medicine).

In other embodiments, the antibody is a fully human monoclonal antibody, such as an IgG2, that binds to ERBB3 and prevents the HRG and EGF-like ligand-induced intracellular phosphorylation of ERBB3.

Anti-ERBB3 antibodies, such as seribantumab, can be generated, e.g., in prokaryotic or eukaryotic cells, using methods well known in the art. In one embodiment, the antibody is produced in a cell line capable of glycosylating proteins, such as CHO cells. Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246: 1275-1281 (1989).

### III. Pharmaceutical Compositions

Pharmaceutical compositions suitable for administration to a patient are typically in forms suitable for parenteral administration, e.g., in a in liquid carrier, or suitable for reconstitution into liquid solution or suspension, for intravenous administration.

In general, compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions (e.g., comprising an anti-ERBB3 antibody). Liquid compositions for parenteral administration can be formulated for administration by injection or continuous infusion. Routes of administration by injection or infusion include intravenous, intraperitoneal, intramuscular, intrathecal and subcutaneous. In one embodiment, the anti-ERBB3 antibody is administered intravenously (e.g., over the course of one hour).

Seribantumab is supplied as a sterile clear liquid solution in single-use vials for injectable use at a concentration of 25 mg/mL (1,000 mg per 40 mL vials; 250 mg per 10 mL vials).

### IV. Patient Populations

Provided herein are effective methods for treating a subject (i.e., a human subject) having a tumor that comprises an NRG1 fusion gene using an anti-ERBB3 antibody according to a particular dosage regimen.

In one embodiment, a human patient for treatment using the subject methods has a locally advanced or metastatic solid tumor comprising an NRG1 fusion gene, e.g., as assessed by a tumor biopsy or liquid biopsy assay, including molecular assays, such as PCR, NGS (RNA or DNA) or FISH testing.

In another embodiment, the subject has a locally advanced or metastatic solid tumor. In another embodiment, the subject has an advanced refractory solid tumor. Non-limiting examples of cancers for treatment include squamous cell carcinoma, lung cancer (e.g., invasive mucinous adenocarcinoma (IMA), small-cell lung cancer, non-small cell lung cancer, squamous non-small cell lung cancer (NSCLC), non-squamous NSCLC), glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, pancreatic ductal adenocarcinoma (PDAC), glioblastoma (glioblastoma multiforme), cervical cancer, stomach cancer, bladder cancer, gallbladder cancer (GBC), hepatoma, breast cancer, colon carcinoma, and head and neck cancer (or carcinoma), diffuse large B-cell lymphoma (DLBCL), neuroendocrine tumor of the nasopharynx, gastric cancer, germ cell tumor, sarcoma, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, gastroesophageal junction (GEJ) cancer, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)). In one embodiment, the subject has IMA. In another embodiment, the subject has ovarian cancer.

In another embodiment, the subject has a tumor that comprises an NRG1 fusion wherein the NRG1 fusion comprises a gene (i.e., a fusion partner) selected from the group consisting of, but not limited to: DOC4, CLU, STMN2, PCM1, CD74; SLC3A2; SDC4; ATP1B1; ROCK1; FOXA1; AKAP13; THBS1; PDE7A; THAP7; SMAD4; RAB3IL1; PMEPA1; STMN2; SLC3A2; VAMP2; RBPMS; WRN; RAB2IL1; SARAF; APP; KIF13B; INTS9; ADAM9; CDH1; COX10-AS1; DIP2B; DPYSL2; GDF15; HMBOX1; MDK; MRPL13; NOTCH2; PARP8; POMK; SETD4; TNC; TSHZ2; VTCN1; WHSC1L1; and ZMYM2.

Patients can be tested or selected for one or more of the above described clinical attributes prior to, during, or after treatment.

### V. Treatment Protocols

Provided herein are methods for treating a tumor in a human patient, wherein the tumor comprises an NRG1 fusion gene, by administering to the patient an anti-ERBB3 antibody according to a particular clinical dosage regimen (i.e., at a particular dose amount and according to a specific dosing schedule). In one embodiment, methods for treating a subject (e.g., human patient) having a tumor that comprises an NRG1 fusion gene are provided, wherein the method comprises administering to the subject a therapeutically effective amount of an ERBB3 (HER3) antibody, wherein the antibody is administered at a once weekly dose of between about 2,000 mg to about 4,000 (e.g., at a dose of 2,000 mg, 2,250 mg, 2,500 mg, 2,750 mg, 3,000 mg, 3,250 mg, 3,500 mg, 3, 750 mg, or 4,000 mg). In one embodiment, the antibody is administered intravenously at a once weekly dose of 3,000 mg.

In another embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of between about 2,000 mg to about 4,000 (e.g., at a dose of 2,000 mg, 2,250 mg, 2,500 mg, 2,750 mg, 3,000 mg, 3,250 mg, 3,500 mg, 3, 750 mg, or 4,000 mg) and wherein the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively.

In another embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of 3,000 mg.

In another embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of 3,000 mg, and wherein the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively.

In another embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of 3,000 mg, and wherein the antibody comprises heavy and light chain variable region amino acid sequences comprising SEQ ID NOs: 2 and 4, respectively.

In another embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of 3,000 mg, and wherein the antibody comprises heavy chain and light chain amino acid sequences comprising SEQ ID NOs: 12 and 13, respectively.

In certain embodiments, the dosage regimen is adjusted to provide the optimum desired response (e.g., an effective response). For example, in some embodiments, administration of the antibody once weekly is discontinued if it is insufficient to effect treatment (e.g., as evidenced by clinical disease progression, increased symptoms, and/or no clinical improvement compared to baseline). A determination that administration once weekly is insufficient to effect treatment can be made by any suitable means. In one embodiment, the determination is assessed by radiographic assessment (e.g., via computerized tomography (CT), positron emission tomography (PET) and/or magnetic resonance imaging (MRI)). In another embodiment, the determination is assessed by “Response Evaluation Criteria in Solid Tumors” (RECIST) version 1.1 guidelines. In another embodiment, the determination is assessed by liver function test (LFT). In another embodiment, the determination is assessed by one or more disease (e.g., tumor) markers (e.g., carbohydrate antigen (CA19-9), cancer embryonic antigen (CEA), cancer antigen 125 (CA-125), and/or cancer antigen 15-3 (CA 15-3).

In another embodiment, the treatment is discontinued for up to three weeks if the subject experiences a clinically significant adverse event (e.g., Grade ≥3). An exemplary clinically significant adverse event includes, but is not limited to, hematologic toxicity (e.g., febrile neutropenia, neutropenic infection, Grade 4 neutropenia >7 days, Grade ≥3 thrombocytopenia for >7 days, Grade ≥3 thrombocytopenia with clinically significant bleeding, Grade 4 thrombocytopenia, and Grade ≥3 anemia >7 days). Another exemplary clinically significant adverse event is non-hematologic toxicity (e.g., (1) Grade ≥3 nausea, vomiting, or diarrhea lasting more than 72 hours despite optimal medical support with anti-emetics or anti-diarrheals, (2) Grade 4 (life-threatening) vomiting, or diarrhea, irrespective of duration, (3) any other grade ≥3 adverse event, except Grade ≥3 fatigue and anorexia lasting for <7 days or Grade ≤2 infusion related reactions).

In another embodiment, the once weekly antibody dose is reduced upon resuming treatment after the subject experience a clinically significant adverse event (e.g., Grade ≥3). For example, the once weekly antibody dose is reduced by 5%, 10%, 15%, 20%, 25%, or 30% upon resuming treatment after the subject experiences a clinically significant adverse event. In one embodiment, the once weekly antibody dose is reduced by 25% upon resuming treatment after the subject experiences a clinically significant adverse event. In another embodiment, the once weekly antibody dose is reduced to 2,750 mg, 2,500 mg, 2,250 mg, 2,000 mg, 1,750 mg, or 1,500 mg upon resuming treatment after the subject experiences a clinically significant adverse event. In one embodiment, the once weekly antibody dose is reduced to 2,250 mg upon resuming treatment after the subject experiences a clinically significant adverse event.

In another embodiment, the once weekly antibody dose is reduced by 50% upon resuming treatment after the subject experiences two or more clinically significant adverse events (e.g., Grade ≥3). For example, the once weekly antibody dose is reduced by 5%, 10%, 15%, 20%, 25%, 30%, 35%, 40%, 45%, 50%, 55%, 60%, 65%, or 70% upon resuming treatment after the subject experiences two or more clinically significant adverse events. In one embodiment, the once weekly antibody dose is reduced by 50% upon resuming treatment after the subject experiences two or more clinically significant adverse events. In another embodiment, the once weekly antibody dose is reduced to 2,250 mg, 2,000 mg, 1,750 mg, 1,500 mg, 1,250 mg, 1,000 mg, 750 mg, or 500 mg upon resuming treatment after the subject experiences two or more clinically significant adverse events. In one embodiment, the once weekly antibody dose is reduced to 1,500 mg upon resuming treatment after the subject experiences two or more clinically significant adverse events.

In one embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of 3,000 mg, and wherein the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively, and wherein the once weekly antibody dose is reduced by 25% or more (e.g., reduced to 2,750 mg, 2,500 mg, 2,250 mg, 2,000 mg, 1,750 mg, or 1,500 mg) upon resuming treatment after the subject experiences a clinically significant adverse event.

In one embodiment, methods for treating a subject having a tumor that comprises an NRG1 fusion gene are provided, wherein the method comprises administering to the subject a therapeutically effective amount of an ERBB3 (HER3) antibody, wherein the antibody is administered at a once weekly dose of 3,000 mg, and wherein the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively, and wherein the once weekly antibody dose is reduced by 50% or more (e.g., reduced to 2,250 mg, 2,000 mg, 1,750 mg, 1,500 mg, 1,250 mg, 1,000 mg, 750 mg, or 500 mg) upon resuming treatment after the subject experiences two or more clinically significant adverse events.

In another aspect, methods for treating a subject (e.g., human patient) having a tumor that comprises an NRG1 fusion gene are provided, wherein the method comprises administering to the subject a therapeutically effective amount of an ERBB3 (HER3) antibody, wherein the antibody is administered at a once weekly dose of between about 2,000 mg to about 4,000 (e.g., at a dose of 2,000 mg, 2,250 mg, 2,500 mg, 2,750 mg, 3,000 mg, 3,250 mg, 3,500 mg, 3,750 mg, or 4,000 mg). For example, in one embodiment, the antibody is administered at a dose of 2,000 mg once a week. In another embodiment, the antibody is administered at a dose of 2,250 mg once a week. In another embodiment, the antibody is administered at a dose of 2,500 mg once a week. In another embodiment, the antibody is administered at a dose of 2,750 mg once a week. In another embodiment, the antibody is administered at a dose of 3,000 mg once a week. In another embodiment, the antibody is administered at a dose of 3,250 mg once a week. In another embodiment, the antibody is administered at a dose of 3,550 mg once a week. In another embodiment, the antibody is administered at a dose of 3,750 mg once a week. In another embodiment, the antibody is administered at a dose of 4,000 mg once a week. In another embodiment, the antibody is administered at a once weekly dose of between about 2,000 mg to about 4,000 (e.g., at a dose of 2,000 mg, 2,250 mg, 2,500 mg, 2,750 mg, 3,000 mg, 3,250 mg, 3,500 mg, 3,750 mg, or 4,000 mg) until intolerance (e.g., unmanageable toxicity). In another embodiment, the antibody is administered at a once weekly dose of between about 2,000 mg to about 4,000 (e.g., at a dose of 2,000 mg, 2,250 mg, 2,500 mg, 2,750 mg, 3,000 mg, 3,250 mg, 3,500 mg, 3,750 mg, or 4,000 mg) until progressive disease (PD). In one embodiment, the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively. In another embodiment, the antibody comprises VH and VL regions comprising the amino acid sequences set forth in SEQ ID NOs: 2 and 4, respectively. In another embodiment, the antibody comprises a HC and LC comprising the amino acid sequences set forth in SEQ ID Nos: 12 and 13, respectively.

The anti-ERBB3 antibody can be administered to a subject by any suitable means. For example, in one embodiment, the antibody is administered intravenously. In another embodiment, the antibody is administered intravenously over about one hour.

The treatment methods described herein can be continued for as long as clinical benefit is observed or until unmanageable toxicity or disease progression occurs. For example, in one embodiment, the treatment is continued for 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, or three years or more.

### VI. Combination Therapy

As herein provided, anti-ERBB3 antibodies (e.g., seribantumab) can be co-administered with a second therapeutic agent to effect improvement in subjects having a tumor that comprises an NRG1 fusion gene. In one embodiment, the second therapeutic agent is a targeted therapeutic, such as a small molecule inhibitor or an antibody, e.g., against ERBB2 (HER2), ERBB3, ERBB4, EGFR, IGF1-R, C-MET, Lewis Y, MUC-1, EpCAM, CA125, prostate specific membrane antigen (PSMA), PDGFR-α, PDGFR-β, C-KIT, or an FGF receptor. For example, in one embodiment, the second therapeutic agent is the antibody is FTN002 (also referred to as MM-111) which targets the HER2/HER3 pathway (see, e.g., PCT/US2012/029292, the contents of which are expressly incorporated herein by reference).

As used herein, co-administration (combined administration) includes simultaneous administration of the compounds in the same or different dosage form, or separate administration of the compounds (e.g., sequential administration). For example, the anti-ERBB3 antibody (e.g., seribantumab) can be simultaneously administered with the second therapeutic agent (e.g., a small molecule inhibitor or a second antibody), wherein both the antibody and the second agent are formulated together. Alternatively, the anti-ERBB3 antibody can be administered in combination with the second agent, wherein both the antibody and the second agent are formulated for separate administration and are administered concurrently or sequentially. For example, the antibody can be administered first followed by the administration of the second agent, or vice versa. Such concurrent or sequential administration preferably results in both seribantumab and the second therapeutic agent being simultaneously present in treated patients.

In another embodiment, the methods described herein can be utilized in combination (e.g., simultaneously or separately) with another treatment, e.g., radiation, surgery, immunotherapy (e.g., monoclonal antibodies and tumor-agnostic treatments (such as checkpoint inhibitors), oncolytic virus therapy, T-cell therapy, and/or cancer vaccines), chemoimmunotherapy (e.g., one or more drugs to kill or slow the growth of cancer cells combined with treatments to stimulate or restore the ability of the immune system to fight cancer), or chemotherapy (e.g., camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, 5-fu, or camptothecin+apo21/TRAIL (a 6× combo)), one or more proteasome inhibitors (e.g., bortezomib or MG132), one or more Bc1-2 inhibitors (e.g., BH3I-2′ (bcl-xl inhibitor), indoleamine dioxygenase-1 inhibitor (e.g., INCB24360, indoximod, NLG-919, or F001287), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002; 8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., Avastin), synthetic triterpenoids (see Hyer et al., Cancer Research 2005; 65:4799-808), c-FLIP (cellular FLICE-inhibitory protein) modulators (e.g., natural and synthetic ligands of PPARγ (peroxisome proliferator-activated receptor γ), 5809354 or 5569100), kinase inhibitors (e.g., Sorafenib), Trastuzumab, Cetuximab, Temsirolimus, mTOR inhibitors such as rapamycin and temsirolimus, Bortezomib, JAK2 inhibitors, HSP90 inhibitors, PI3K-AKT inhibitors, Lenalildomide, GSK3β inhibitors, IAP inhibitors and/or genotoxic drugs.

The methods described herein can further be used in combination with one or more anti-proliferative cytotoxic agents. Classes of compounds that may be used as anti-proliferative cytotoxic agents include, but are not limited to, the following:

Alkylating agents (including, without limitation, nitrogen mustards, ethylenimine derivatives, alkyl sulfonates, nitrosoureas and triazenes): Uracil mustard, Chlormethine, Cyclophosphamide (CYTOXAN™) fosfamide, Melphalan, Chlorambucil, Pipobroman, Triethylenemelamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Suitable anti-proliferative agents for combining with the methods described herein, include without limitation, taxanes, paclitaxel (paclitaxel is commercially available as TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone B1, [17]-dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone B10, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5(10)-trien-3-ol, cyclostreptin, isolaulimalide, laulimalide, 4-epi-7-dehydroxy-14,16-didemethyl-(+)-discodermolides, and cryptothilone 1, in addition to other microtubuline stabilizing agents known in the art.

In cases where it is desirable to render aberrantly proliferative cells quiescent in conjunction with or prior to treatment with the methods described herein, hormones and steroids (including synthetic analogs), such as 17a-Ethinylestradiol, Diethylstilbestrol, Testosterone, Abiraterone, Enzalutamide, Androgen receptor degraders (ARDs), Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX™, or anti-estrogens (e.g., fulvestrant, non-steroidal aromatase inhibitor (letrozole, anastrozole), steroidal aromatase inhibitor (exemestane), and novel selective estrogen receptor degraders (SERDs), and selective estrogen receptor modulators (SERMs)) can also be administered to the patient. When employing the methods or compositions described herein, other agents used in the modulation of tumor growth or metastasis in a clinical setting, such as antimimetics, can also be administered as desired.

Methods for the safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto. The chemotherapeutic agent(s), immunotherapeutic agent(s), chemoimmunotherapeutic agent(s) and/or radiation therapy can be administered according to therapeutic protocols well known in the art. It will be apparent to those skilled in the art that the administration of such agent(s) and/or radiation therapy can be varied depending on the disease being treated and the known effects of the agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

In another embodiment, the methods described herein further comprise inhibition (antagonism) of MET signaling pathway activity. Exemplary MET inhibitors include, but are not limited to: Crizotinib, PHA-665752, SU11274, SGX-523, BMS-777607, JNJ-38877605, Tivantinib, PF-04217903, MGCD-265, Capmatinib, AMG 208, MK-2461, AMG 458, NVP-BVU972, and Tepotinib.

In another embodiment, the methods described herein further comprise inhibition (antagonism) of mTOR (mammalian target of rapamycin) signaling pathway activity. The term “mTOR” refers to the protein mammalian target of rapamycin, which is a serine/threonine kinase related to the PI3K family and is a downstream effector of the PI3K/AKT signaling pathway. mTOR functions as a regulator of cell growth and metabolism, and exists in two complexes, mTORC1 and mTORC2.

Accordingly, in one embodiment, the methods described herein further comprise administration of an mTOR inhibitor. In one embodiment, the mTOR inhibitor inhibits mTORC1. In another embodiment, the mTOR inhibitor inhibits mTORC2. In yet another embodiment, the mTOR inhibitor inhibits both mTORC1 and mTORC2. mTOR inhibitors are well known in the art and include, for example, gedatolisib, sirolimus, everolimus, temsirolimus, dactolisib, AZD8055, ABTL-0812, PQR620, GNE-493, KU0063794, torkinib, ridaforolimus, sapanisertib, voxtalisib, torin 1, torin 2, OSI-027, PF-04691502, apitolisib, GSK1059615, WYE-354, vistusertib, WYE-125132, BGT226, palomid 529, WYE-687, WAY600, GDC-0349, XL388, bimiralisib (PQR309), omipalisib (GSK2126458, GSK458), onatasertib (CC-223), samotolisib, omipalisib, RMC-5552, and GNE-477.

In another embodiment, the methods described herein further comprise administration of a RET inhibitor. In another embodiment, the methods described herein further comprise administration of a KRAS G12C inhibitor. In another embodiment, the methods described herein further comprise administration of an NTRK inhibitor. In another embodiment, the methods described herein further comprise administration of an EGFR inhibitor. In another embodiment, the methods described herein further comprise administration of an ALK inhibitor. In another embodiment, the methods described herein further comprise administration of a MEK inhibitor. In another embodiment, the methods described herein further comprise administration of an ERK inhibitor. In another embodiment, the methods described herein further comprise administration of an AKT inhibitor. In another embodiment, the methods described herein further comprise administration of a PI3K inhibitor.

In another embodiment, the methods described herein further comprise administration of one or more anti-estrogens, including, but not limited to, fulvestrant, non-steroidal aromatase inhibitor (letrozole, anastrozole), steroidal aromatase inhibitor (exemestane), and novel selective estrogen receptor degraders (SERDs), and selective estrogen receptor modulators (SERMs)).

### VII. Outcomes

With respect to target lesions, responses to therapy may include:

Complete Response (CR): Disappearance of all target lesions. Any pathological lymph nodes (whether target or non-target) must have reduction in short axis to <10 mm;

Partial Response (PR): At least a 30% decrease in the sum of the diameters of target lesions, taking as reference the baseline sum diameters;

Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression); and

Stable Disease (SD): Neither sufficient shrinkage to qualify for PR nor sufficient increase to qualify for PD, taking as reference the smallest sum diameters while on study. (Note: a change of 20% or less that does not increase the sum of the diameters by 5 mm or more is coded as stable disease). To be assigned a status of stable disease, measurements must have met the stable disease criteria at least once after study entry at a minimum interval of 6 weeks.

With respect to non-target lesions, responses to therapy may include:

Complete Response (CR): Disappearance of all non-target lesions and normalization of tumor marker level. All lymph nodes must be non-pathological in size (<10 mm short axis). If tumor markers are initially above the upper normal limit, they must normalize for a patient to be considered in complete clinical response;

Non-CR/Non-PD: Persistence of one or more non-target lesion(s) and/or maintenance of tumor marker level above the normal limits; and

Progressive Disease (PD): Appearance of one or more new lesions and/or unequivocal progression of existing non-target lesions. Unequivocal progression should not normally trump target lesion status. It must be representative of overall disease status change, not a single lesion increase.

In exemplary outcomes, patients treated according to the methods disclosed herein may experience improvement in at least one sign of responsiveness to treatment. For example, in one embodiment, the patient so treated exhibits CR, PR, or SD. In another embodiment, the patient so treated experiences tumor shrinkage and/or decrease in growth rate, i.e., suppression of tumor growth. In another embodiment, unwanted cell proliferation is reduced or inhibited. In yet another embodiment, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other embodiments, such improvement is measured by a reduction in the quantity and/or size of measurable tumor lesions. Measurable lesions are defined as those that can be accurately measured in at least one dimension (longest diameter is to be recorded) as ≥10 mm by CT scan (CT scan slice thickness no greater than 5 mm), 10 mm caliper measurement by clinical exam or ≥20 mm by chest X-ray. The size of non-target lesions, e.g., pathological lymph nodes can also be measured for improvement. In one embodiment, lesions can be measured on chest x-rays or CT or MRI films.

In other embodiments, cytology or histology can be used to evaluate responsiveness to a therapy. The cytological confirmation of the neoplastic origin of any effusion that appears or worsens during treatment when the measurable tumor has met criteria for response or stable disease can be considered to differentiate between response or stable disease (an effusion may be a side effect of the treatment) and progressive disease.

In some embodiments, administration of an effective amount of the anti-ERBB3 antibody according to any of the methods provided herein produce at least one therapeutic effect selected from the group consisting of reduction in size of a tumor, reduction in number of metastatic lesions appearing over time, complete remission, partial remission, stable disease, increase in overall response rate, or a pathologic complete response. In some embodiments, the provided methods of treatment produce a comparable clinical benefit rate (CBR=CR+PR+SD≥6 months) better than that achieved without administration of the anti-ERBB3 antibody. In other embodiments, the improvement of clinical benefit rate is about 20% 20%, 30%, 40%, 50%, 60%, 70%, 80% or more.

### VIII. Kits and Unit Dosage Forms

Also provided are kits that include a pharmaceutical composition containing an anti-ERBB3 antibody, such as seribantumab, and a pharmaceutically-acceptable carrier, in a therapeutically effective amount adapted for use in the preceding methods. The kits can optionally also include instructions, e.g., comprising administration schedules, to allow a practitioner (e.g., a physician, nurse, or patient) to administer the composition contained therein to administer the composition to a patient having a tumor that comprises an NRG1 fusion gene. In one embodiment, the kit further comprises instructions for use. In another embodiment the kit includes a syringe.

Optionally, the kits include multiple packages of the single-dose pharmaceutical composition(s) each containing an effective amount of the antibody (e.g., seribantumab) for a single administration in accordance with the methods provided above. Optionally, instruments or devices necessary for administering the pharmaceutical composition(s) may be included in the kits. For instance, a kit may provide one or more pre-filled syringes containing an amount of seribantumab that is about 100 times the dose in mg/kg indicated for administration in the above methods.

The following examples are merely illustrative and should not be construed as limiting the scope of this disclosure in any way as many variations and equivalents will become apparent to those skilled in the art upon reading the present disclosure.

All patents, patent applications and publications cited herein are incorporated herein by reference in their entireties.

### EXAMPLES

### Example 1

Novel patient-derived and isogenic models of NRG1-rearranged cancers were developed and used to examine the effect of seribantumab on growth, apoptosis, and intracellular signaling in vitro and in vivo, as discussed in detail below.

1. Materials and Methods Patient-derived cell lines and xenografts were developed under institutional review board approved biospecimen protocols and written informed consent was obtained from patients for collection of tumor material. Mice were cared for, and experiments were conducted in accordance with a protocol approved by the Memorial Sloan Kettering Cancer Center (New York, N.Y.) Institutional Animal Care and Use Committee and Research Animal Resource Center.

The LUAD-0061AS3 PDX model was generated from samples obtained from a patient with an SLC3A2-NRG1 fusion-driven lung cancer. The patient exhibited disease progression while on treatment with afatinib (40 mg/day) at the time of sample collection. A thoracentesis was performed and pleural effusion fluid sample was obtained. Heparin was added to a final concentration of 1 mg/L fluid. All cells were isolated by centrifugation (300×g, 5 minutes, in a tabletop centrifuge) and red blood cells were removed by incubating for 5 minutes in ACK (ammonium-chloride-potassium) Lysis Buffer (Thermo Fisher Scientific, A1049201). A total of 20×10⁶ cells were then implanted into the subcutaneous flank of 6-week-old female NSG (NOD/SCID gamma) mice (Envigo). The LUAD-0061AS3 cell line was generated from LUAD-0061AS3 PDX tumor tissue obtained after seven serial passages. Briefly, fresh tumors were cut into small pieces and then digested in a cocktail of tumor dissociation enzymes obtained from Miltenyi Biotec (130-095-929) in 5 mL serum-free DME:F12 media for 1 hour at 37° C., with vortexing every 5-10 minutes. Digested samples were resuspended in 45 mL complete growth media to inactivate the dissociation enzymes, and then cells were pelleted by centrifugation. Finally, cells were plated in complete growth media and allowed to propagate over multiple generations in the absence of afatinib, trypsinized when necessary to subculture, and eventually only single cells remained. The OV-10-0050 PDX model was established from a surgically resected clinical sample with a CLU-NRG1 fusion (CLU exon 8 fused to NRG1 exon 6) by WuXi AppTec (Drilon A, et al., Cancer Discov 2018; 8:686-95). PDX tumors were serially transplanted three times before a model was considered established.

The breast cancer epithelial cell lines, MDA-MB-175-VII (cata-log no. HTB-25, RRID: CVCL_1400) and MCF-7 (catalog no. HTB-22, RRID: CVCL_0031), were obtained from the ATCC. MDA-MB-175-VII cells express a DOC4-NRG1 fusion (Drilon A, et al. 2018 and Trombetta D, et al., Oncotarget 2018; 9:9661-71). MCF-7 cells were derived from pleura effusion isolated from a patient with breast cancer and are estrogen receptor positive (Bowtell D D, et al., Nat. Rev. Cancer 2015; 15:668-79). This cell line has been profiled by the Broad Institute Depmap program and does not have any NRG1 rearrangements (Mitra A K, et al., Gynecol. Oncol. 2015; 138:372-7). Human bronchial epithelial cells were immortalized by overexpression of CDK4 and TERT (HBEC-3KT cell line) and were obtained from Dr. John Minna (UT South Western, Dallas, Tex.; Kobel, et al., Int. J. Gynecol. Pathol. 2016; 35:430-41). A p53 C-terminal mutant was introduced into HBEC-3KT (HBECp53) as described previously (Ishikawa F, et al., Blood 2005; 106: 1565-73) and a CD74-NRG1 fusion was expressed in these cells by lentiviral-mediated transduction of the cDNA. Cells expressing the fusion were selected using 200 mg/mL hygromycin. The HBECp53-SLC3A2-NRG1 cells are an unselected population in which the SLC3A2-NRG1 fusion has been introduced by CRISPR-Cas9—mediated genome editing, as we have described previously for ROS1 and BRAF fusions (Cadranel J, et al., Oncologist 2021; 26:7-16 and Geuijen C A W, et al., Cancer Cell 2018; 33: 922-36). HCC-95 cells were obtained from Dr. William Lockwood (BC Cancer Center, Vancouver, British Columbia, Canada, RRID: CVCL_5137) and these cells were found to have NRG1 amplification by whole-exome sequencing (Drilon A, et al., 2018). Cell lines were tested for Mycoplasma every 6 months (MycoAltert Kit, Lonza), with the most recent testing conducted 6 months prior to completion of the studies described herein. Authenticated cell lines purchased from the ATCC 1 year prior to the studies were expanded and stocks were frozen. A new vial of cells was thawed and used for 10-15 passages (every 2 months) and the known oncogene was verified by RT-PCR each time. The identity of cell lines that were created was routinely confirmed by testing for the known oncogene fusion.

The MDA-MB-175-VII cell line was maintained in DMEM: Ham F12 (1:1) medium supplemented with 20% FBS. For experiments, MDA-MB-175-VII cells were plated and grown in DMEM: Ham F12 medium containing 10% FBS. MCF-7 cells were grown in DMEM supplemented with 10% FBS. HBECp53 cells were grown in KSM supplemented with bovine pituitary extract and EGF. Isogenic HBECp53 cell lines expressing NRG1 fusions were grown in DMEM: Ham F12 (1:1) medium supplemented with 10% FBS. HCC-95 cells were grown in RPMI 1640 medium supplemented with 10% FBS. All growth media were supplemented with 1% antibiotic (penicillin/FNJ-026 streptomycin mixture). Cells were subcultured using trypsin (0.25%)/EDTA (1 mmol/L) when stock flasks reached 75% confluency and replated at a 1:3 dilution. Cells were kept in a humidified incubator infused with 5% CO2 and maintained at 37° C.

For the time-course experiments, cells were plated at a density of 5,000 (HCC-95) or 10,000 (all others) cells per well in 12-well tissue culture plates, and then treated 24 hours later (time 0) with the respective agents. For the MCF-7 growth assay, cells were treated with 1 mmol/L seribantumab for 1 hour prior to incubation with 10 ng/mL NRG1-b1. Cells were trypsinized and counted at the relevant time-point shown on the graphs. For dose-response studies, cells were plated at a density of 7,500-10,000 cells in white clear-bottom 96-well plates in a volume of 90 mL complete growth media and 10 mL chemicals added at 10× concentration (to achieve 1× concentration) in a final volume of 100 mL. After 96-hour incubation, 10 mL AlamarBlue cell viability reagent was added to achieve a final concentration of 10%. AlamarBlue is a cell permeable pH-sensitive dye that is reduced when it enters the mitochondria and emits fluorescence at a different wavelength (Gloeckner H, J. Immunol. Methods 2001; 252:131-8). Fluorescence was measured (excitation, 530 nm and emission, 585 nm) using a Molecular Dynamics Spectramax M2 Fluorescence Plate Reader, as described previously (Somwar R, J. Biomol. Screen 2009; 14:1176-84). In each experiment, background fluorescence was determined in cells treated with 1 mmol/L of the 20S proteasome inhibitor, carfilzomib, which is toxic to most cells at high concentrations, and was subtracted from all values. There were 3-4 replicates of each condition. Relative IC50 values and 95% confidence interval values were determined by nonlinear regression analysis using GraphPad Prism 8 software using either a variable slope model or in cases where inhibition was only partial, a three-parameter fit was used. The curve fitting resulted in R2 >0.8 for the datasets. Each condition was assayed in triplicate in 2-5 independent experiments.

Crushed PDX tumor samples were mixed with Matrigel (50%) and injected into the subcutaneous flank of 6-week-old female NSG (LUAD-0061AS3) or Balb/c nude (OV-10-0050) mice. When tumors reached approximately 100-150 mm3, mice were randomly assigned to groups of 5-8 and treatment was commenced. There were 2 mice per group with bilateral flank tumors for the protein phosphorylation/expression study in the LUAD-0061AS3 PDX model. Drugs were administered once, and then tumors were collected at 2, 24, and 168 hours posttreatment. Afatinib was administered by oral gavage once daily as a suspension (in 0.5% methylcellulose-0.4% Tween-80) on a 5-days-on and 2-days-off schedule. Seribantumab was administered in PBS by injection into the peritoneal cavity once every 3 days for a twice weekly dosing (BIW) schedule. Mice were observed daily throughout the treatment period for signs of morbidity and mortality. Tumor length and width and animal weights were measured twice weekly. Tumor volume was calculated using the empirical formula V=length×width 2×0.52. The percentage change in tumor volume of each tumor was calculated using the formula [(V2−V1)/V1)]×100, where V1 is the starting tumor volume and V2 is the final tumor volume.

For detection of the SLC3A2-NRG1 fusion transcript, RNA was extracted using a Qiagen RNA Mini Kit and cDNAs were synthesized using SuperScript IV VILO (Thermo Fisher Scientific) according to the manufacturer's instructions. The SLC3A2-NRG1 fusion was detected by RT-PCR using 5′-ATGCTTGCTGGTGC-CGTGGTCA-3′ (forward, SLC3A2 exon 4) and 5′-GGTCTTTCAC-CATGAAGCACTCCCC-3′ (reverse, NRG1 exon 6) primers. For detection of the CD74-NRG1 fusion, forward primers targeting CD74 exon 6 (5′-AGAGCTGGATGCACCATTGG-3′) were used. For detection of the CLU-NRG1 fusion, forward primer targeting CLU (5′-TGAAGACTCTGCTGCTGTTTGTG-3′) and two reverse primers targeting NRG1 (R1: 5′-GTTTTCTCCTTCTCCGCACA-TTT and R2: 5′-TATCTCGAGGGGTTTGAAAGGTC-3′) were used. For expression of NRG1 splice variants by qPCR, TaqMan Gene Expression Master Mix was used (Thermo Fisher Scientific, 4369016) with the following expression assays: NRG1a (Hs01103794_m1), NRG1b (Hs00247624_m1), and GAPDH (Hs02786624_G1). NRG1 mRNA levels are expressed relative GAPDH mRNA level. All cell line values were normalized to the HBECp53 cells.

Histology and IHC were performed as described previously (Liu Z, Clin. Cancer Res. 2015; 21:1752-63). Briefly, xenograft tissues were collected, fixed in 4% buffered formalin saline at room temperature for 24 hours, embedded in paraffin blocks, and then sections of 4 mm thickness were mounted on glass slides. After deparaffinization, the tissue sections were subjected to hematoxylin and eosin (H&E) staining, or antigen retrieval for IHC staining. For IHC assays, slides were immersed in 3% H2O2 for 5 minutes, washed, and then blocked for 15 minutes in 5% BSA. Slides were incubated in primary antibodies overnight at 4° C., washed, and then incubated with biotinylated anti-rabbit secondary antibody using a Diamino-benzidine (DAB) Kit (Dako) for 30 minutes at 37° C. The positive signals from IHC staining were detected using a DAB Detection Kit according to the manufacturer's instructions. Slides were stained with antibodies against WT1 (6F-H2, Dako), p53 (318-6-11, Dako), phospho-HER3 Y1289 (21D3, Cell Signaling Technology), and TTF-1 (8G7G3/1, Dako), and counterstained with hematoxylin.

Tumor datasets were compared by two-way ANOVA, with Dunnett or Tukey multiple comparison test to determine significance. P<0.05 was considered a statistically significant difference between two values or datasets. All statistical analyses were conducted using GraphPad Prism 8 software (RRID: SCR_002798). The AUC was calculated by the trapezoid rule (Gagnon R C, J. Pharmacokinet. Biopharm. 1998; 26:87-102) and groups were compared using one-way ANOVA. Caspase 3/7 activity was compared using Student t test. All experiments consisted of 2-3 replicates per condition and data are expressed as mean±SD or SEM.

2. Results
a. Expression of NRG1 Alpha and Beta Isoforms in Patient-Derived Cell Lines with NRG1 Alterations

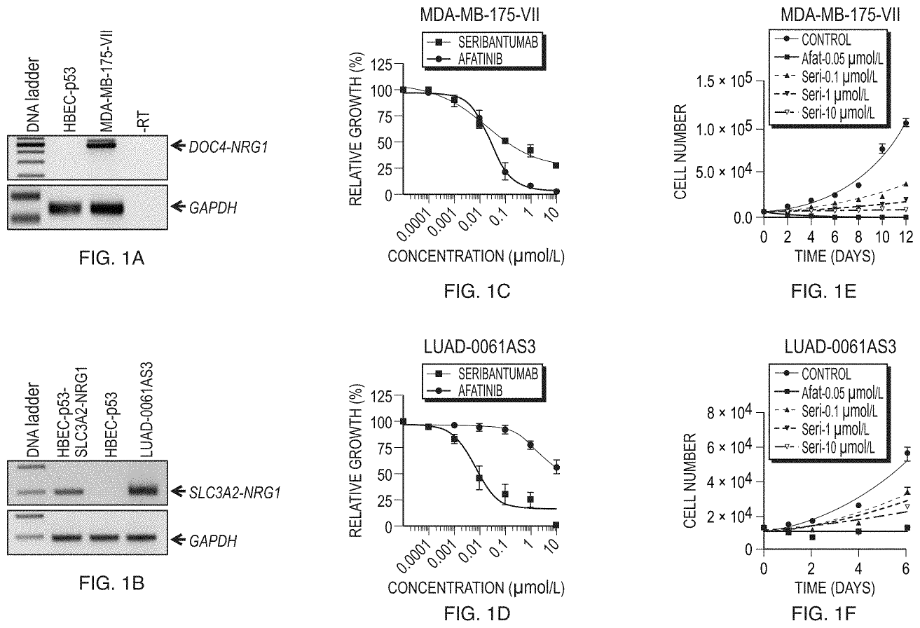

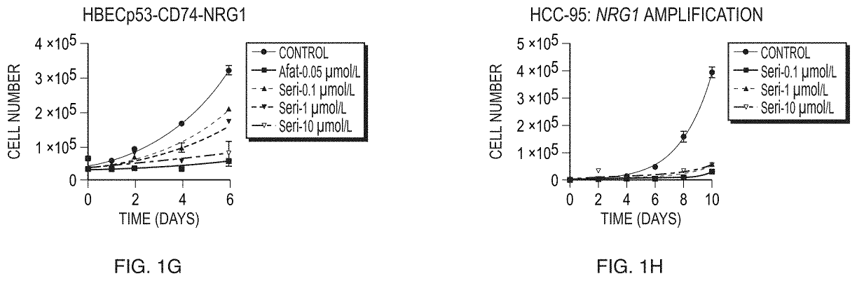

Oncogenic NRG1 fusions retain only a small part of NRG1 and this portion invariably includes the EGF-like domain. This domain in NRG1 exists in two forms, namely the alpha and beta isoforms. To comparatively assess the expression level of NRG1 in the different cell lines, the EGF-like domain was the focus, as this is required for transformation and used isoform-specific qPCR assays. Cancer cell lines with NRG1 fusion or NRG1 amplification were compared with cells without an NRG1 alteration. This was achieved by qPCR analysis using TaqMan assays that were specific for each of the alpha and beta splice variants of NRG1. The breast cancer cell line, MDA-MB-175-VII, harbors a chromosomal translocation between NRG1 and DOC4 and the lung cancer cell line, LUAD-0061AS3, harbors a translocation between NRG1 and SLC3A2. Expression of the DOC4-NRG1 and SLC3A2-NRG1 fusions in the cell lines was confirmed by RT-PCR (FIGS. 1A and B). The HCC-95 cell line is a lung cancer cell line that has amplification of NRG1. For comparison, the MCF-7 breast cancer cell line was used and HBECp53 cell line (untransformed immortalized human bronchiolar epithelial cells); neither cell lines are known to harbor any NRG1 alterations. All cell lines expressed NRG1a and NRG1β mRNAs at varying levels. The mRNA level in each cell line was expressed relative to corresponding mRNA in HBECp53 cells. The MCF-7 cells were found to have the lowest expression of NRG1 isoforms. HCC-95 cells expressed very high levels of NRG1a and NRG1β mRNA, likely due to the NRG1 amplification. Whereas HCC-95 cells had the highest level of NRG1a mRNA expression compared with cell lines with NRG1 fusions and the control cells, the LUAD-0061AS3 cell line had the highest level of NRG1β mRNA. HCC-95 cells had 14-fold more NRG1β mRNA than the MDA-MB-175-VII cells. These results suggest that cell lines with NRG1 alterations express both NRG1 isoforms. However, the limited number of cell lines analyzed suggests that caution should be exercised in interpreting these results.

b. Seribantumab Inhibits Growth of Cells Harboring NRG1 Alterations

Cells expressing NRG1 fusions rely on activation of HER3 for growth and survival. Here, the ability of seribantumab to inhibit the growth of two cell lines that harbor NRG1 rearrangements (MDA-MB-175-VII, DOC4-NRG1 fusion and LUAD-0061AS3, SLC3A2-NRG1 fusion) was evaluated in comparison with tumor and nontumor cell lines without an NRG1 fusion (MCF-7 and HBECp53, respectively). Treatment of the two NRG1 fusion-positive cell lines with seribantumab or afatinib reduced growth in a dose-dependent manner (FIGS. 1C and D). Both seribantumab and afatinib had minimal effect on the growth of MCF-7 breast cancer cells or HBECp53 cells. The estimated IC₅₀ values obtained for growth inhibition are determined. MDA-MB-175-VII (IC₅₀=0.02 μmol/L) and LUAD-0061AS3 (IC₅₀=1.4 μmol/L) cells were approximately 2,260- and 32.3-fold more sensitive to seribantumab than MCF-7 cells (IC₅₀=45.2 μmol/L), respectively. Similarly, MDA-MB-175-VII and LUAD-0061AS3 cells were approximately 10,000- and 145-fold more sensitive to seribantumab than the nontumor HBECp53 cells (IC₅₀=203 μmol/L).

To further explore the temporal nature of cell growth inhibition by seribantumab, cells were treated for up to 12 days with vehicle, seribantumab (0.1, 1, and 10 μmol/L), or afatinib (0.05 μmol/L), and then proliferation was estimated. In these experiments, the MDA-MB-175 and LUAD-0061AS3 cell lines, isogenic HBECp53 cells ectopically expressing a CD74-NRG1 fusion, and the NRG1-amplified lung cancer cell line, HCC-95 (FIGS. 1E-H) were used. RT-PCR confirmed the presence of the CD74-NRG1 fusion in the HBECp53-CD74-NRG1 cells Seribantumab slowed the growth of the MDA-MB-175-V11 cells as early as 24 hours after treatment was initiated, and growth was blocked for the entire 12-day period of the experiment by the 1 and 10 μmol/L concentrations (FIG. 1E). Similar results were obtained with the LUAD-0061AS3 cells (FIG. 1F). Although the HBECp53-CD74-NRG1 cells were less sensitive to seribantumab than LUAD-0061AS3 and MDA-MB-171-VII cells, an almost complete inhibition of growth at the highest concentration of seribantumab (FIG. 1G) was nevertheless observed. The HCC-95 cells were the most sensitive to seribantumab, with growth completely inhibited at the lowest antibody concentration (FIG. 1H). Afatinib treatment (0.05 μmol/L) was also effective at inhibiting growth of the three cell lines with NRG1 rearrangements (FIGS. 1C-G). Afatinib sensitivity of HCC-95 cells was not examined in this study. These results suggest that seribantumab effectively inhibits growth of tumor cell lines that harbor NRG1 fusions or NRG1 amplification.

c. Seribantumab Specifically Inhibits NRG1-Dependent Cell Growth

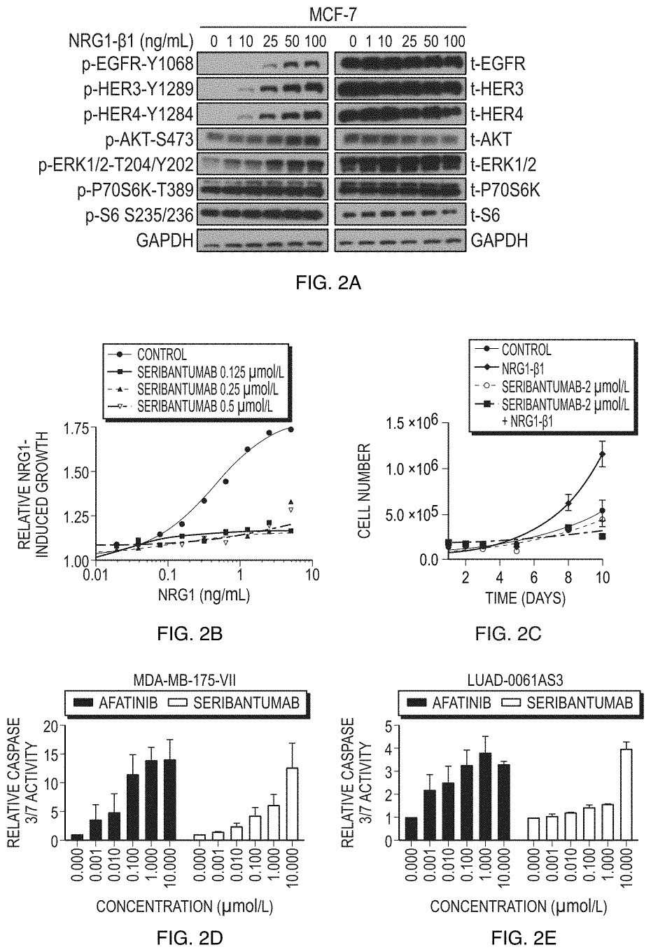

A first goal was to confirm that NRG1 could activate known mitogen-activated pathways in MCF-7 cells. To this end, cells were treated with increasing concentrations of NRG1-β1 (the EGF-like domain) for 10 minutes, and then protein phosphorylation was determined by Western blotting (FIG. 2A). Treatment of MCF-7 cells with NRG1-β1 caused a dose-dependent increase in phosphorylation of EGFR, HER3, and HER4. Increased phosphorylation of the three receptors was observed with as little as 10 ng/mL NRG1-β1, with phosphorylation of EGFR being the least sensitive. This was accompanied by an increase in phosphorylation of AKT, ERK1/2, and elements of the mTOR pathway, including ribosomal protein S6 (FIG. 2A). Next, the ability of seribantumab to block NRG1-stimulated growth of MCF-7 cells was examined. Cells were simultaneously treated with varying concentrations of NRG1-β1 (0-5 ng/mL) and seribantumab (0-0.5 μmol/L) for 96 hours, and then viability was determined. Treatment of MCF-7 cells with NRG1-β1 resulted in a significant increase in cell viability likely because of enhanced proliferation (FIG. 2B). The lowest concentration of seribantumab used (0.125 μmol/L) largely suppressed growth of NRG1-β1-stimulated MCF-7 cells. This was further explored in temporal studies in which MCF-7 cells were pretreated with 2 μmol/L seribantumab for 1 hour prior to addition of 10 ng/mL NRG1-β1 for up to 10 days and growth was assessed. Seribantumab pretreatment prevented NRG1-β1-stimulated growth completely (FIG. 2C). The results demonstrate that inhibiting HER3 with seribantumab effectively blocks NRG1-dependent cell proliferation.

d. Seribantumab Induces Apoptosis in Cells Harboring NRG1 Rearrangements

To examine whether seribantumab can induce cell death, caspase 3/7 enzymatic activity was measured in cell homogenates as a surrogate for apoptosis. MDA-MB-175-VII and LUAD-0061AS3 cells were treated with 0-10 μmol/L seribantumab or afatinib for 48 hours. As a positive control for activation of caspase 3/7, 1 μmol/L carfilzomib was used. A dose-dependent increase in caspase 3/7 activity in cells treated with afatinib or seribantumab was observed (FIG. 2D). Afatinib was more effective at activating caspase 3/7 than seribantumab at lower concentrations in MDA-MB-175-VII. However, at the 10 μmol/L concentration, afatinib and seribantumab were equally effective at activating caspase 3/7 (afatinib, 14.1±3.6-fold above control and seribantumab, 12.7±4.2-fold above control) and comparable with the level of caspase 3/7 activity stimulated by carfilzomib (16.6±1.9-fold above control). Although afatinib and seribantumab stimulated caspase 3/7 activity to a similar extent at the highest concentration used in LUAD-0061AS3 (FIG. 2E), the magnitude of the response was much less than that observed in MDA-MB-175-VII cells (afatinib, 3.3±0.1-fold above control and seribantumab, 4.0±0.3-fold above control). This may reflect a less active apoptosis pathway in LUAD-0061AS3 cells because carfilzomib also stimulated less caspase 3/7 activity in this cell line (5.8±1.3-fold above control) compared with MDA-MB-175-VII cells. These results suggest that seribantumab can induce apoptosis in a dose-dependent manner in NRG1 fusion-positive breast and lung cancer cell lines.

e. Seribantumab Inhibits Phosphorylation of Downstream Mediators in Cells with NRG1 Alterations

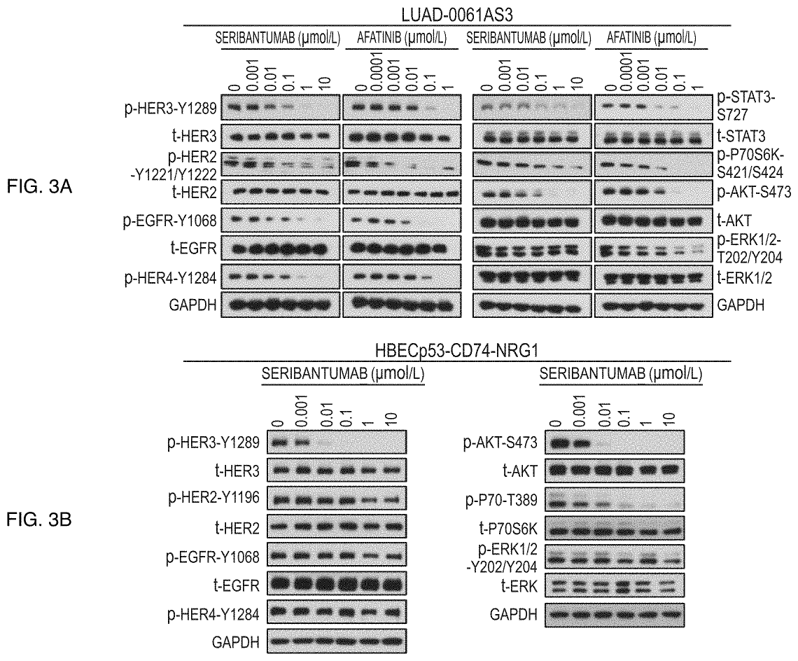

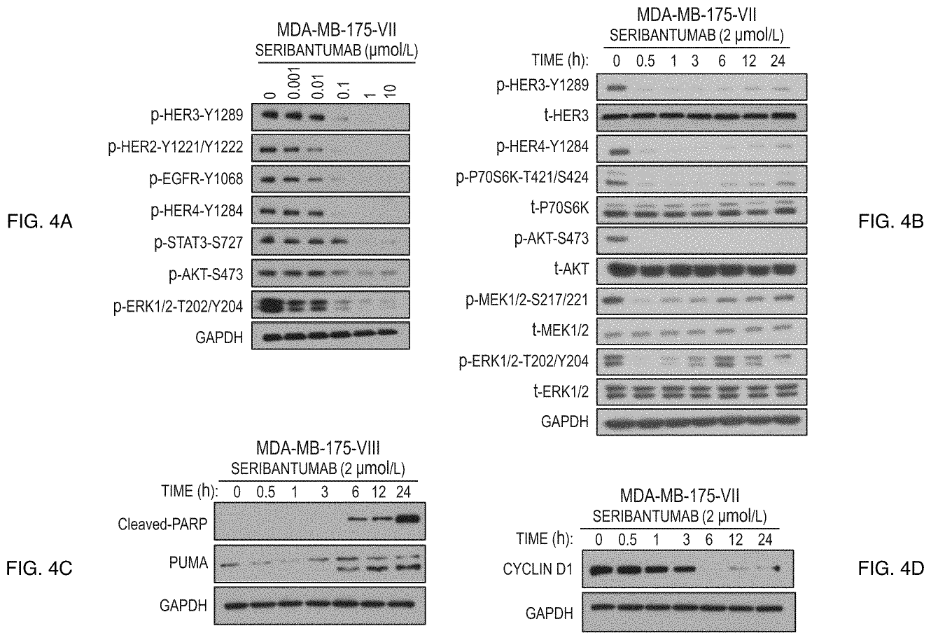

To investigate the cellular signaling networks affected by seribantumab, the phosphorylation states of EGFR, HER2, HER3, HER4, and elements of the PI3K, mTOR, and MAPK pathways were examined by Western blotting, following treatment of serum-starved LUAD-0061AS3, HBECp53-CD74-NRG1, and MDA-MB-175-VII cells with the indicated concentrations of seribantumab (FIGS. 3 and 4A). Treatment of LUAD-0061AS3 cells with seribantumab resulted in almost complete inhibition of phosphorylation of EGFR, HER2, HER3, HER4, AKT, and STAT3 (FIG. 3A). Phosphorylation of ERK1/2 was less sensitive to seribantumab treatment. The inhibitory effect of seribantumab on protein phosphorylation was similar to that obtained with afatinib in most instances (FIG. 3A). In HBECp53-CD74-NRG1 cells, seribantumab treatment completely inhibited HER3 phosphor-ylation and reduced phosphorylation of HER2, EGFR, and HER4 to a lesser extent (FIG. 3B). Similar to observations in LUAD-0061AS3 cells, phosphorylation of AKT, p70S6K, and STAT3 was almost completely inhibited by seribantumab treatment (FIG. 3B). In MDA-MB-175-VII cells, seribantumab fully inhibited phosphorylation of HER3, HER2, EGFR, and HER4 and reduced phosphorylation of AKT, ERK1/2, and STAT3 to a large extent (FIG. 4A). Neither seribantumab nor afatinib had any effect on expression of any protein after the treatment, suggesting that loss of phosphorylation observed in response to seribantumab treatment was due entirely to a block in signal transduction. In HCC-95 cells, seribantumab treatment also inhibited phosphorylation of HER2, HER3, and downstream effectors, with little effect on EGFR phosphorylation. Taken together, these results suggest that treatment with seribantumab can disrupt HER3-dependent signaling, block phosphorylation of ERBB receptors and downstream signaling, reduce expression of cell-cycle proteins, and induce expression of proapoptotic proteins. These events likely culminate in inhibition of growth and impaired survival.

To obtain a more comprehensive understanding of the mechanistic action of seribantumab, the temporal relationship between seribantumab treatment and phosphorylation of signaling proteins or expression of proteins that regulate apoptosis and the cell cycle was evaluated. Serum-deprived MDA-MB-175-VII cells were treated with 2 mmol/L seribantumab for up to 24 hours, and then whole-cell extracts were prepared and subjected to Western blotting. Seribantumab treatment rapidly reduced phosphorylation of HER3, HER4, and downstream signaling, with full inhibition observed 30 minutes after treatment was initiated (FIG. 4B). Phosphorylation of AKT remained completely inhibited for the entire 24-hour treatment period, even though there appeared to be a slight increase in HER3, HER4, and p70S6 kinase phosphorylation at the 12- and 24-hour timepoints. Phosphorylation of MEK1/2 and ERK1/2 was inhibited rapidly, but reactivation was seen earlier than was observed for HER3 and HER4 (FIG. 4B). Expression of the proapoptotic proteins, cleaved-PARP and PUMA, was elevated by seribantumab treatment in a time-dependent manner (FIG. 4C) and remained elevated from 6-24 hours. This is in agreement with observations shown in FIG. 2D, highlighting that seribantumab induced activation of caspase 3/7 by 48 hours of treatment. The level of cyclin D1, a protein that permits transit through the G1-phase of the cell cycle, was reduced in seribantumab-treated cells by 1 hour and was undetectable by 6 hours (FIG. 4D). As observed with phosphor-ylation of some proteins, cyclin D1 level began to be restored by 12 hours (FIG. 4D).

f. Seribantumab Treatment Induces Tumor Regression in NSCLC PDX Model with SLC3A2-NRG1 Rearrangement

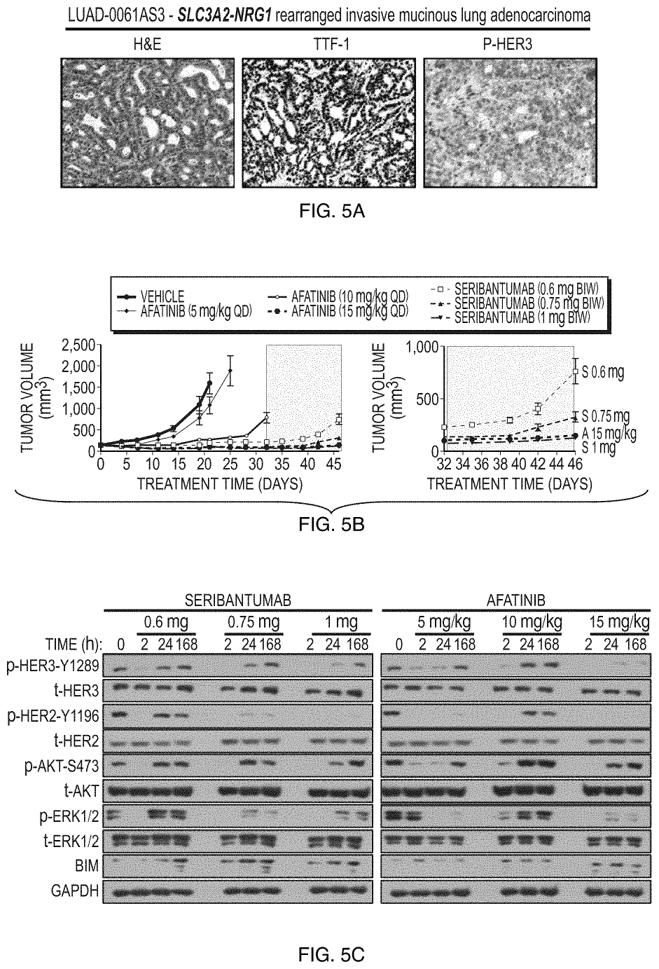

The inhibition of growth of cell lines with NRG1 fusions and NRG1-stimulated MCF-7 cells by seribantumab supported the evaluation of seribantumab efficacy in vivo. An NSCLC PDX model was generated from a patient with invasive mucinous adenocarcinoma harboring an SLC3A2-NRG1 fusion. Histologic characterization of the PDX tumors is shown in FIG. 5A. As expected, the tumor was positive for TTF-1 (lung adenocarcinoma marker) and showed membranous phospho-HER3 staining as demonstrated previously (Trombetta D, et al., Oncotarget 2018; 9:9661-71).

LUAD-0061AS3 PDX tumors were implanted into the subcutaneous flank of immunocompromised mice (seven animals/group) and treatment was initiated 2 weeks later with seribantumab (0.6, 0.75, or 1 mg per dose, twice weekly) or afatinib (5, 10, or 15 mg/kg, once daily). The 5 mg/kg daily dose of afatinib is equivalent to the human dose of 50 mg daily, which is the maximum approved dose for patients. Seribantumab is being evaluated at 3,000 mg weekly in a phase II clinical study (CRESTONE, NCT04383210), which is equivalent to a dose of 11.5 mg two times per week in mice.

The tumor volume as a function of time is illustrated in FIG. 5B, and the AUC computed for each group to facilitate comparison of tumor volume between groups at the last date all groups had surviving animals (day 35) was determined. The 5 mg/kg afatinib dose caused a small, but significant reduction in tumor growth (FIG. 5B). However, higher doses of afatinib and all doses of seribantumab tested caused a bigger decrease in tumor volume (FIG. 5B). Treatment with 0.75 or 1 mg seribantumab resulted in regression of four of seven and six of seven tumors, respectively. Tumors in the 1 mg seribantumab group continued to shrink, resulting in a maximum tumor reduction of 57.2%±2.6% by day 42 and this was maintained for another 2 weeks. The highest dose of afatinib (15 mg/kg) was also effective at causing regression of six of seven tumors in the. However, the maximum response to the 15 mg/kg afatinib dose was not sustained for more than a few days and a slow tumor regrowth was observed while the animals were still receiving treatment. The 1 mg BIW seribantumab dosage, which is lower than the human dosage, was as effective as the 15 mg/kg daily dose of afatinib (FIG. 5B, right). No treatment caused any statistically significant reduction in animal weight or negatively influenced animal health in any noticeable way. Taken together, these results suggest that seribantumab is more effective at reducing tumor growth than afatinib.

g. Seribantumab Treatment Blocks Phosphorylation of Known Growth Modulators and Induces Expression of Apoptosis Markers In Vivo

The data presented above indicate that seribantumab effectively reduced growth and blocked activation of growth-promoting pathways in cancer cell lines with NRG1 fusions and abrogated growth of NRG1 fusion-positive cell lines and LUAD-0061AS3 PDX tumors, irrespective of tissue of origin or fusion partner. Phosphorylation of HER2, HER3, AKT, and ERK1/2 was examined to evaluate the ability of seribantumab and afatinib to interfere with NRG1-dependent signaling. Animals bearing LUAD-0061AS3 PDX tumors were given a single administration of seribantumab (0.6, 0.75, or 1 mg) or afatinib (5, 10, or 15 mg/kg), and then tumors were removed at 2, 24, or 168 hours post-drug administration. Protein phosphorylation was then detected by Western blotting of PDX tumor lysates.

As shown in FIG. 5C (left), all doses of seribantumab resulted in reduced phosphorylation of HER2, HER3, AKT, and ERK1/2 by the 2-hour timepoint, with higher doses being more effective at the longer timepoints. Although there was some reactivation of phosphorylation of HER3, AKT, and ERK1/2 at the later timepoints, HER2 phosphorylation remained inhibited even after 168 hours of treatment at higher doses. Similarly, afatinib reduced phosphorylation of HER2,

HER3, AKT, and ERK1/2, with the best effect seen with the highest dose studied (15 mg/kg). With the exception of HER2, reactivation of protein phosphorylation was observed at the later timepoints (FIG. 5C, right). At 5 mg/kg in mice, a dose that is equivalent to that used clinically (mouse to human dose equivalency is estimated allometrically using FDA guidelines), afatinib was able to inhibit HER2 phosphorylation completely by 2 hours and caused a major loss in HER3 phosphorylation.

Next, the ability of seribantumab and afatinib to induce expression of the proapoptotic protein, BIM, in the same tumor lysates probed above for protein phosphorylation was examined. Induction of BIM expression was clearly seen after 24 hours of treatment with all doses of seribantumab. Tumors isolated from mice treated with 0.75 and 1 mg seribantumab had higher levels of BIM than vehicle-treated tumors by 2 hours after drug administration. Elevated BIM was present at the 168-hour timepoint following administration of all doses of seribantumab studied. Only the higher doses of afatinib were able to induce sustained BIM expression, but to a lesser degree than that elicited by seribantumab (FIG. 5C, right). Importantly, the clinically relevant dose of afatinib (5 mg/kg once daily in mice) caused a small, but transient increase in BIM level.

h. Seribantumab Treatment Induces Complete Tumor Regression in HGSOC PDX Model with CLU-NRG1 Rearrangement

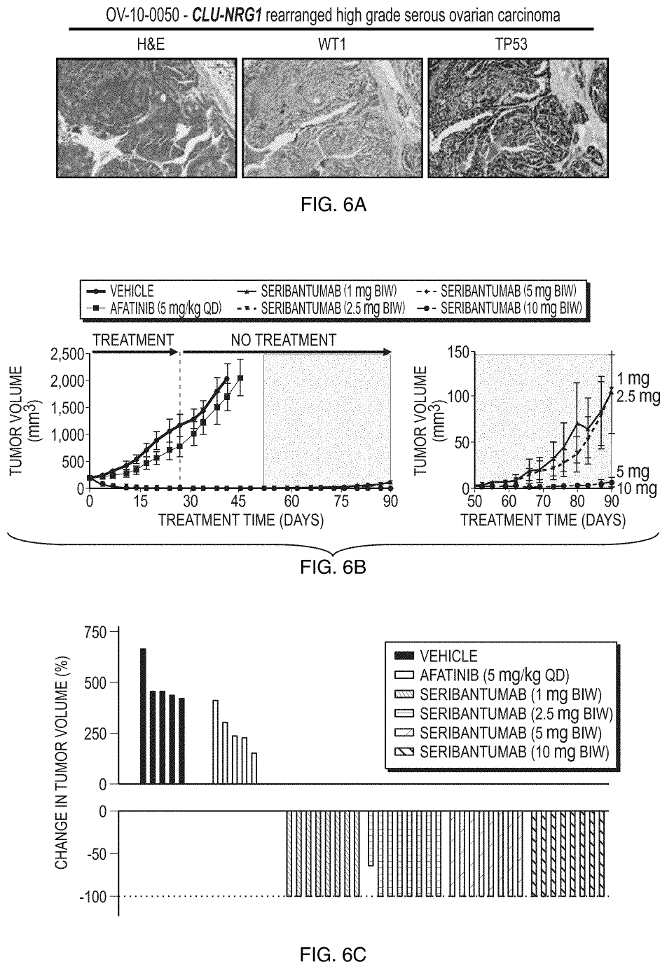

High-grade serous ovarian cancer (HGSOC) accounts for 70%-80% of ovarian cancer related deaths, and overall survival has not changed significantly for several decades (Bowtell D D, Nat. Rev. Cancer 2015; 15:668-79). Seribantumab was shown previously to block growth of xenograft tumors generated from OVCAR8 cells (Sheng Q, et al., Cancer Cell 2010; 17:298-310), which exhibit HGSOC histology (Mitra A K, et al., Gynecol. Oncol. 2015; 138:372-7). Here, the efficacy of seribantumab in an ovarian PDX model (0V-10-0050), which was derived from a surgically resected ovarian tumor and harbors a CLU-NRG1 fusion, was examined. RT-PCR confirmed the presence of the CLU-NRG1 fusion. Xenograft tissue morphology and IHC markers (positive for WT1 and strong nuclear staining for TP53) were consistent with HGSOC histology (FIG. 6A; Kobel M, et al., Int. J. Gynecol. Pathol. 2016; 35:430-41). Mice bearing OV-10-0050 PDX tumors (5-8 animals/group) were treated with 1, 2.5, 5, or 10 mg seribantumab (twice weekly) or 5 mg/kg afatinib (once daily) and tumor growth was assessed. As with the above experiments in the NSCLC PDX model, the doses of seribantumab used here were lower than the dose that is used in patients. Treatment was terminated at day 27 and tumor growth was monitored for an additional 63 days (90 days after initiation of treatment or once tumors reached the maximum allowable size). The tumor volume as a function of time is illustrated in FIG. 6B, and the AUC was computed for each group to compare tumor volumes between groups at the last date of treatment. Afatinib treatment caused a small, but significant decrease in tumor growth (P=0.003; FIG. 6B). Seribantumab administration rapidly inhibited growth of OV-10-0050 PDX tumors, leading to significant tumor shrinkage at all doses tested (FIG. 6B). The average tumor volumes at the time of the last treatment were: 983.7±254.5 (vehicle); 786.4±190.5 (afatinib); 1.3±0.3 (1 mg seribantumab); 1.9±0.6 (2.5 mg seribantumab); 17.9±14.5 (5 mg seribantumab); and 2.1±0.6 (10 mg seribantumab), and are illustrated in FIG. 6C as the percentage change in tumor size. Following treatment cessation, tumors that were previously treated with seribantumab continued to shrink, while tumors in the vehicle- and afatinib-treated groups continued to grow (FIG. 6B, right). Forty-one days after treatment began, mice bearing vehicle- and afatinib-treated tumors were sacrificed because of the high tumor burden. By day 73 (46 days after treatment termination), tumors started to regrow in the 1, 2.5 and 5 mg seribantumab groups. However, only one of eight tumors started to regrow in the two highest dose groups at the end of the study, suggesting that seribantumab likely eliminated the vast majority of tumor cells. No treatment caused any significant change in overall animal health or weight.

3. Discussion

The NRG1 fusion gene encodes a chimeric protein that engages HER3 to drive tumorigenesis irrespective of histology, and therefore targeting HER3 for therapy of NRG1 fusion-positive cancers constitutes a rational therapeutic strategy that can be exploited. Currently, no FDA-approved therapy for patients with NRG1 fusion-driven cancers exists. The only HER3-specific targeted agent in clinical trials for this group of malignancies is the monoclonal anti-HER3 antibody, seribantumab.

By utilizing novel disease models that represent NRG1 fusion-driven lung, breast, and ovarian cancers (each with a different NRG1 fusion partner), the effect of seribantumab on growth, apoptosis, and activation state of signaling molecules that regulate proliferation, cell-cycle progression, and survival was analyzed. As demonstrated herein, anti-HER3 antibody (seribantumab) is able to block activation of all ERBB family members in NRG1 fusion-positive cell lines, similar to observations with afatinib. This prominent blockade of the ERBB family resulted in loss of downstream activation of the PI3K-AKT, mTOR, and ERK pathways, culminating in a significant reduction of proliferation and induction of apoptosis. In cells in culture, afatinib was more effective at inhibiting growth than seribantumab. The reason for this difference is unclear. It is possible that the presence of other growth factors in culture media may dampen the in vitro efficacy of seribantumab.

In two in vivo PDX models, seribantumab administration, at a dosage lower than that used in human trials, led to substantial tumor regression of more than 50% in a NSCLC PDX model and 100% regression in the PDX model of HGSOC harboring a CLU-NRG1 fusion. HGSOC accounts for 70%-80% of ovarian cancer-related deaths (Bowtell D D, et al., Nat. Rev. Cancer, 2015; 15:668-79). In the HGSOC model, tumor growth was largely repressed for 63 days after treatment was stopped and animals were monitored for tumor regrowth. In contrast to the effectiveness of seribantumab, a 5 mg/kg afatinib daily dose (human equivalent dose of 50 mg daily), was a poor antagonist of tumor growth, despite full inhibition of HER2 phosphorylation (indicating that tumor penetration of afatinib was not an issue). These observations were contrary to the in vitro results, where afatinib was more effective at blocking cell growth. It is unlikely that the higher efficacy of seribantumab observed in the in vivo studies is due to any antibody-dependent cell-mediated cytotoxicity (ADCC) activity because the NSG strain of mice lacks mature T cells, B cells, and natural killer cells (Ishikawa F, et al., Blood 2005; 106: 1565-73), precluding any ADCC-mediated effects. Instead, the higher in vivo potency of seribantumab could be partially attributed to the sustained increase in expression of proapoptotic proteins, such as BIM, in PDX tumors, compared with afatinib. Afatinib (5 mg/kg once daily) administration did not induce BIM expression in PDX tumors. The lack of cell death may contribute to the poor response to afatinib seen in the two PDX models in this study and in clinical reports where there have been stable disease, short duration of response, or no response in the majority of cases reported. These results suggest that specific inhibition of HER3 with the mAb, seribantumab, in a tumor agnostic fashion should be explored as a therapy for NRG1 fusion-dependent cancers.

One major drawback of preclinical studies attempting to develop therapies for NRG1 fusion-driven cancers is a lack of patient-derived models. Most studies employ either murine NIH-3T3 cells or cancer cell lines in which NRG1 fusions are artificially expressed. In this study, patient-derived breast, lung, and ovarian cancer cell lines and PDX models were used to assess the efficacy of seribantumab. The preclinical results presented here demonstrate that seribantumab is an effective therapeutic agent for cancers arising from NRG1 rearrangements, perhaps aided by its ability to block activation of all ERBB family members, thereby inhibiting the cell cycle and inducing apoptosis. Importantly, it was shown that seribantumab can block growth of a lung cancer cell line with NRG1 amplification. It is possible that NRG1 amplification may emerge as a molecularly defined cancer subset as more diagnostic platforms begin to profile for NRG1 alterations. These novel models can be used to thoroughly compare other potential therapies for cancers with NRG1 fusions, such as the HER2-HER3 bispecific antibody, MCLA-128, and other HER3 antibodies, to be able to better recognize the best-in-class drugs.

In sum, seribantumab was effective at blocking NRG1-stimulated growth of MCF-7 cells. In cells with endogenous NRG1 fusions, blockade of HER3 with seribantumab reduced activation of other ERBB family members (HER2, HER4, and EGFR) and the PI3K-AKT-mTOR, RAS-MAPK, and STAT3 pathways. Importantly, seribantumab blocked growth and induced apoptosis in NRG1 fusion models derived from breast, lung, and ovarian cancers in vitro and in vivo. Stated another way, seribantumab, reduces growth and induces apoptosis in disease models derived from three different histologic cancer subtypes with NRG1 rear-rangements at dosages that are clinically achievable and lower than the human dosage. These results provide a clear preclinical rationale for a tumor agnostic trial of seribantumab to treat NRG1 gene fusion-positive solid tumors.

### Example 2

Oncogenic rearrangements of the neuregulin 1 gene (NRG1) consist of a 5′ partner fused to a 3′ NRG1 sequence that retains the epidermal growth factor (EGF)-like domain, and are found in approximately 0.2% of solid tumors including lung, breast, and gastrointestinal (GI) cancers (Jonna S. et al., Clin. Cancer Res. 2019; 25:4865-4867). Carcinomas of GI origin, including pancreatic and cholangiocarcinoma, represent around 20% of solid tumors harboring NRG1 fusions and there is no approved therapy for this group of cancers (Jonna S. et al., J. Clin. Oncol. 2020; 38(15_suppl):3113). The chimeric NRG1 oncoproteins bind to human epidermal growth factor receptor 3 (HER3/ERBB3) leading to trans-activation of other ERBB family members and trigger a signaling cascade that culminates in oncogenesis. Although targeting HER3 represents a rational therapeutic strategy for cancers harboring NRG1 fusions, this has remained relatively unexplored for GI malignancies with NRG1 alterations. In this study, the efficacy of the anti-HER3 monoclonal antibody seribantumab in preclinical models of NRG1-driven GI cancers was investigated.

Models of isogenic pancreatic cancer cells with NRG1 fusions by lentiviral-mediated cDNA expression of ATP1B1-NRG1 and SLC3A2-NRG1 fusions in immortalized human pancreatic ductal cells (H6c7) were developed. Seribantumab efficacy was evaluated in isogenic cell lines and in patient-derived xenograft (PDX) models of pancreatic adenocarcinoma (CTG-0943, APP-NRG1 fusion) and intrahepatic cholangiocarcinoma (CH-07-0068, RBPMS-NRG1 fusion). Western blotting analysis was used to evaluate protein phosphorylation and expression. The presence of NRG1 fusions was confirmed by reverse transcription polymerase chain reaction (RT-PCR) and next-generation sequencing (NGS).

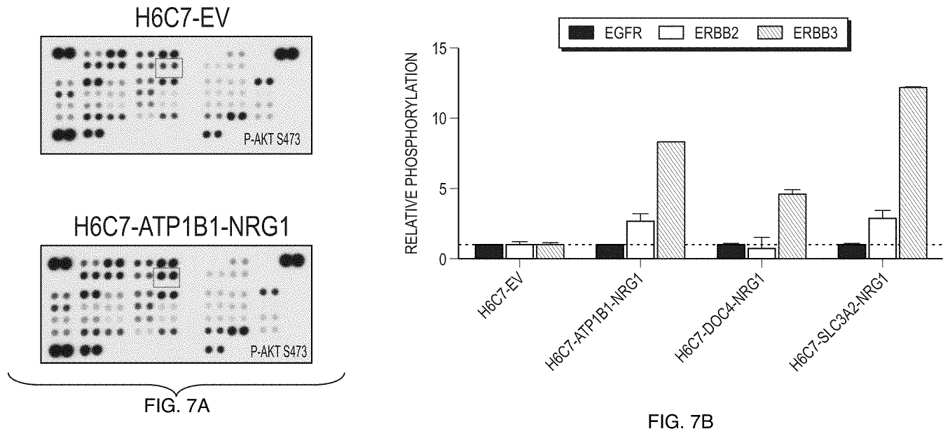

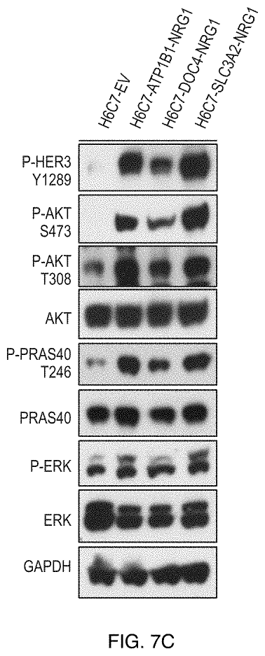

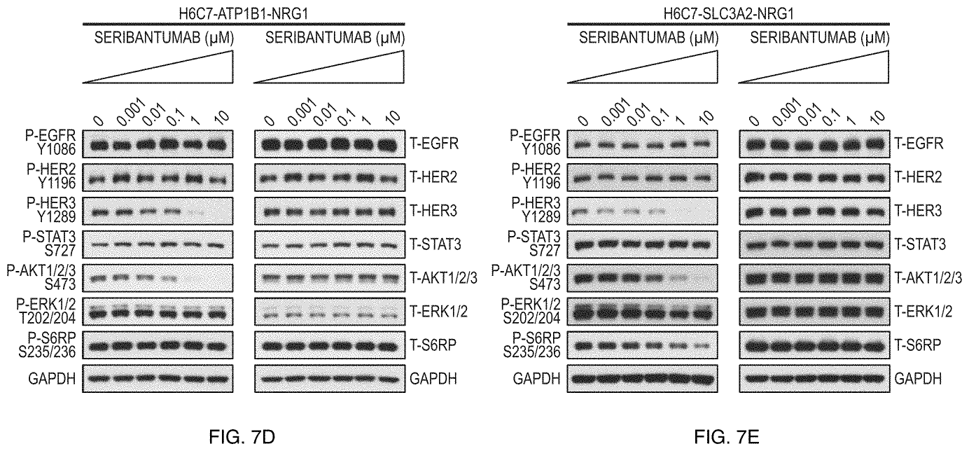

Expression of NRG1 fusions in H6c7 cells resulted in enhanced phosphorylation of HER3 and AKT when compared with empty vector control cells (H6c7-EV). Treatment of H6c7-ATP1B1-NRG1 and H6c7-SLC3A2-NRG1 pancreatic cells with seribantumab resulted in a dose-dependent inhibition of HER3 and AKT phosphorylation (FIGS. 7A-7E). Tumor growth inhibition was observed after administration of 5 mg or 10 mg twice weekly [BIW] seribantumab to a PDX mouse model of pancreatic adenocarcinoma with an APP-NRG1 rearrangement (CTG-0943). The two doses of seribantumab were more effective than afatinib (5 mg/kg QD), a pan-ERBB inhibitor, in this model, causing tumor shrinkage of up to 55% (23-77% range). There was no regression of afatinib-treated pancreatic PDX tumors. After treatment, residual CTG-0943 tumors were extracted for Western blotting analysis (day 24 for vehicle, and day 31 or 32 for the seribantumab and afatinib-groups, respectively). Loss of phosphorylated and total EGFR, HER2 and HER3, cyclin D1, etc. in seribantumab-treated tumors at the end of the study suggests loss of the majority of human tumor cells in the xenograft tumors. This was confirmed using a human-specific GAPH antibody.

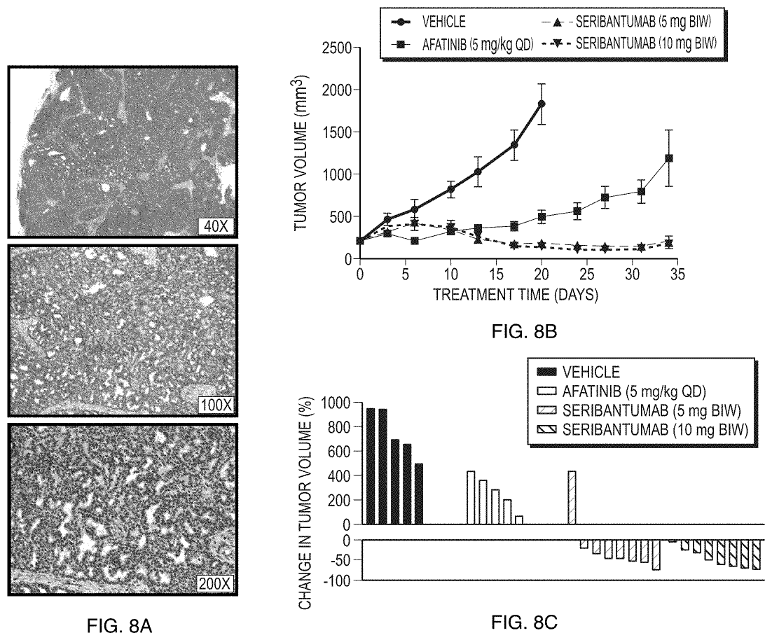

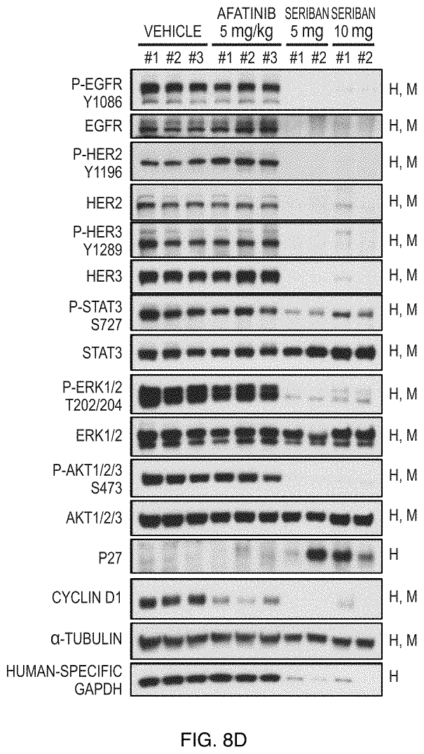

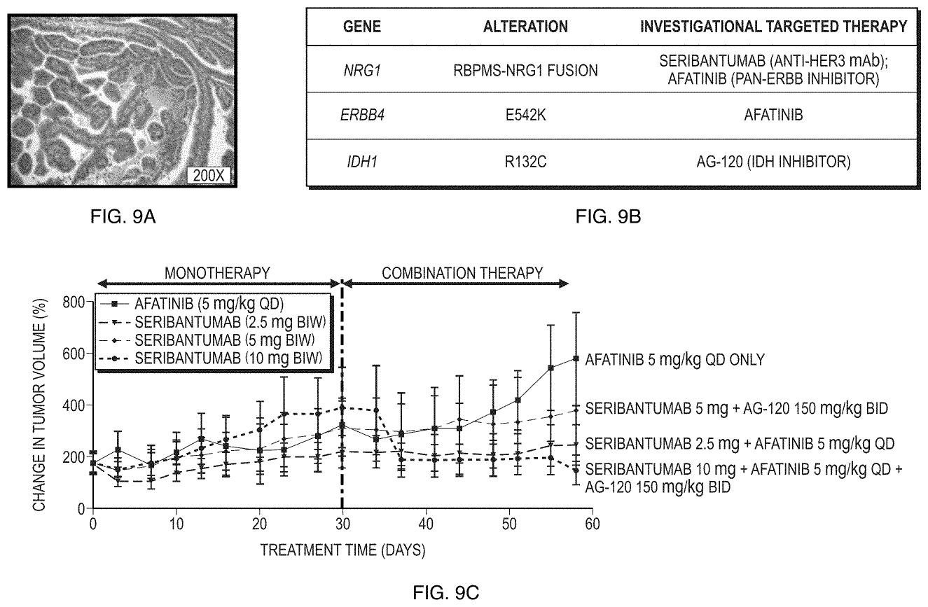

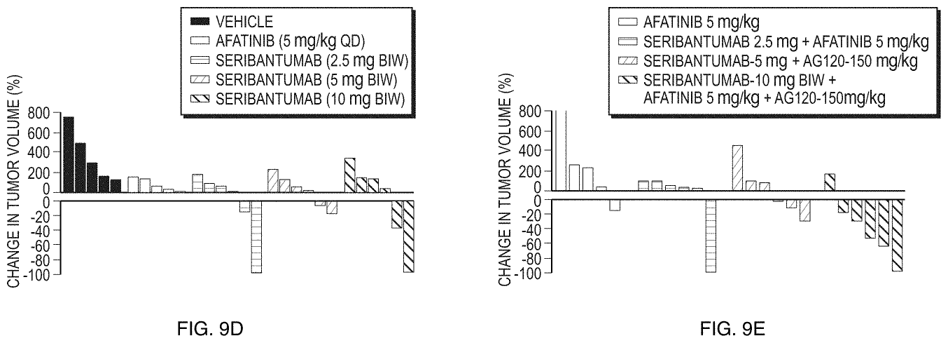

Seribantumab was further evaluated in an intrahepatic cholangiocarcinoma PDX model with an RPBMS-NRG1 fusion (FIGS. 8A-8D), as well as mutations in ERBB4 and IDH1 (CH-17-0068) (FIGS. 9A-9E). While monotherapy seribantumab (5 mg and 10 mg per dose, BIW) was equally effective as afatinib (5 mg/kg once daily [QD]) in this model, enhanced tumor regression was observed with combination therapy. The triple combination of seribantumab 10 mg BIW with afatinib and AG-120, an IDH inhibitor, led to regressions in the majority of tumors. Allometric scaling (based on FDA guidelines) indicates that 5 mg/kg afatinib in mice is equivalent to a human dose of approximately 50 mg daily.

In sum, NRG1 fusions are rare but recurrent oncogenic drivers in GI cancers (see, e.g., Jonna S. et al., Clin. Cancer Res. 2019; 25:4865-4867 and Jonna S. et al., J. Clin. Oncol. 2020; 38(15_suppl):3113). Overexpression of NRG1 fusions in immortalized human pancreatic ductal epithelial H6C7 cells activated HER3 and AKT. Seribantumab inhibits HER3 and AKT phosphorylation in H6C7 cells with NRG1 fusions. Treatment of NRG1 fusion-positive pancreatic PDX model with seribantumab inhibits tumor growth at clinically achievable doses. Residual tumor xenografts show depleted human tumor cell content when assessed by Western blotting. Investigation of a cholangiocarcinoma PDX model with three genomic alterations (NRG1 fusion, and ERBB4 and IDH1 mutations) suggests that treatment of NRG1 fusion-driven tumors harboring additional oncogenic drivers may require combination therapy to address the contribution of each genomic alteration in disease progression. These data support the use of monotherapy seribantumab to treat GI and other cancers uniquely driven by an NRG1 fusion in the ongoing phase 2 CRESTONE study (NCT #04383210).

### Example 3

Comprehensive genomic profiling (CGP) can reveal targetable oncogenic drivers to inform treatment options beyond traditional chemotherapeutics based purely on tumor histology and origin. This is a case series from the Cancer Molecular Screening and Therapeutics (MoST) program, which employs molecular screening of patients with advanced solid tumors of any histology to identify potential actionable mutations and corresponding biomarker-drive therapies. The objective of this study was to illustrate how genomic findings can offer novel therapeutic opportunities and improve outcomes for patients with treatment-refractory gastrointestinal (GI) cancer.

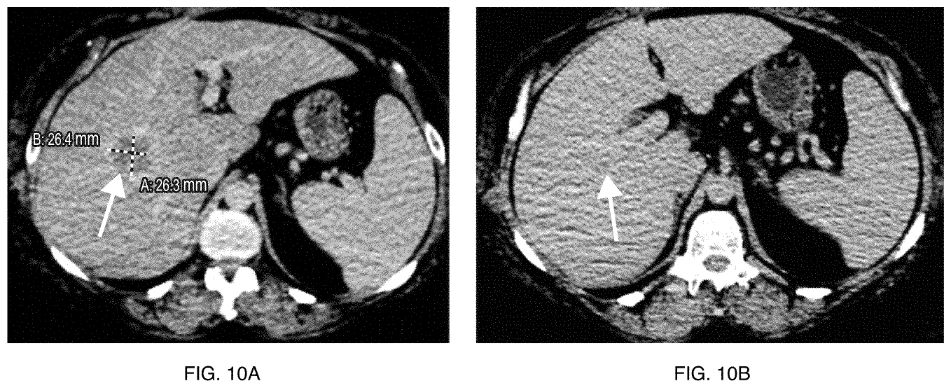

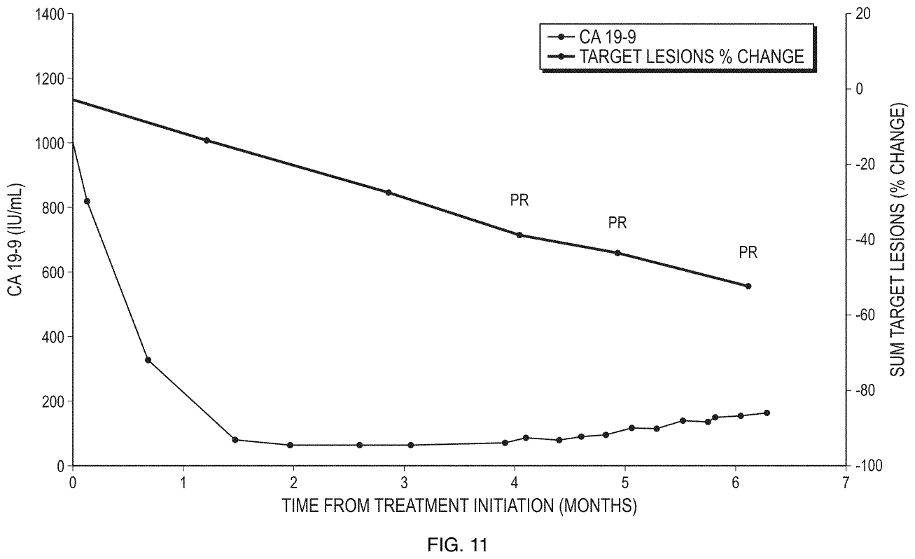

In brief, patients with advanced solid tumors of any histology undergo molecular screening. Genomic alterations are reviewed at molecular tumor boards to identify appropriate biomarker-matched therapeutic sub-studies. Seven GI cancer patients with durable clinical benefit were identified by case review, including a patient with KRAS WT pancreatic cancer harboring an ATP1B1-NRG1 gene fusion. Neuregulin 1 (NRG1) gene fusion proteins are an important oncogenic driver, enriched amongst KRAS wild-type PDAC (8-10%) (Aguirre A J., Clin. Cancer Res. 2019 August; 25(15)). NRG1 is the predominant ligand of ERBB3. These fusion proteins drive tumor progression through aberrant ERBB3 activation. Identification of a 38-year-old female with de novo KRAS wt pancreatic ductal adenocarcinoma (with liver metastases) with ATP1B1-NRG1 gene fusion permitted compassionate access to seribantumab. The patient had previously exhausted available therapies and had been treated as follows: (1) October 2019 to January 2020, FOLFIRINOX, (2) January 2020 to June 2020, FOLFIRI, (3) June 2020 to August 2020, Gemcitabine/Abraxane, (4) August 2020 to December 2020, FOLFIRI. In December 2020, she was treated with seribantumab given her tumor harbored an NRG1 fusion. Treatment with seribantumab resulted in a partial response and ongoing treatment for 8 months at the time of data cutoff for presentation (FIG. 10A, FIG. 10B, and FIG. 11).

In conclusion, CGP can identify rare, but therapeutically relevant genomic alterations with the potential to improve clinical outcomes for advanced, GI cancer patients. Future research should focus on how best to identify patients who will derive the greatest benefit from this precision oncology approach.

### Example 4

A Phase 2 clinical study of seribantumab (referred to as “CRESTONE, Protocol Version 4.0”) is conducted in adult patients with neuregulin-1 (NRG1) fusion positive locally advanced or metastatic solid tumors.

1. Objectives

The primary objective of the study is to determine the Objective Response Rate (ORR) by independent radiologic review to single agent seribantumab in patients with confirmed NRG1 gene fusion positive advanced cancer according to “Response Evaluation Criteria in Solid Tumors” (RECIST 1.1; see, e.g., Eisenhauer, E. et al., (2009), “New response evaluation criteria in solid tumors: revised RECIST guideline (version 1.1),” European Journal of Cancer (Oxford, England: 1990), 45(2), 228-47)).

Secondary objectives of the study include (1) determining the overall efficacy of single agent seribantumab in NRG1 gene fusion positive patients with various advanced cancers through the assessment of the following clinical outcome parameters (e.g., Duration of Response (DoR), Progression-free Survival (PFS), Overall Survival (OS), and Clinical Benefit Rate (Complete Response (CR), Partial Response (PR), and Stable Disease (SD) >24 weeks)) and (2) describing the safety profile of seribantumab in NRG1 gene fusion positive patients.

Exploratory objectives include evaluating (1) the pharmacokinetics of the seribantumab dosing schedule in patients with NRG1 gene fusion positive advanced solid tumors and (2) if mechanistically linked exploratory biomarkers from tumor tissue or blood samples correlate with clinical outcomes.

2. Study Design

This study is an open-label, international, multi-center, Phase 2 study in adult patients with recurrent, locally-advanced or metastatic solid tumors, which harbor the NRG1 gene fusion based upon local testing. Patients have locally advanced or metastatic solid tumors that have progressed after one or more prior standard therapies and for which no available curative therapy exists.

All patients are determined to be NRG1 gene fusion positive based on local testing of tumor tissue per local laboratory directed analyses prior to initiating further screening procedures. After all screening procedures and determination of eligibility for study treatment have been completed, eligible patients are assigned to the appropriate cohort, based upon prior treatment history and local NRG1 gene fusion testing results. Patients are assigned to Treatment Cohorts as follows:

Cohort 1: A minimum of 55 patients with centrally confirmed NRG1 gene fusions who are ERBB/HER2/HER3 treatment-naïve AND harbor NRG1 gene fusions with an EGF-like domain intact

Cohort 2: Up to 10 patients with NRG1 gene fusions with an EGF-like domain intact, who have progressed after prior standard therapy, including prior ERBB/HER2/HER3 directed treatment

Cohort 3: Up to 10 patients with NRG1 fusions without an EGF-like domain (including but not limited to NRG1-PMEPA1, NRG1-STMN2, PCM1-NRG1 and INTS9-NRG1); patients with other NRG1 alterations (i.e., rearrangements); patients with NRG1 fusions and other molecular aberrations lacking standard treatment options; AND patients unable to provide sufficient tissue for central confirmation of NRG1 gene fusion status.

More than 10 patients can enroll under Cohorts 2 and 3 upon approval.

One cycle of treatment consists of 28 days. Dosing begins at the Cycle 1 Week 1 (C1W1) visit. Treatment for all patients assigned to Cohorts 1, 2 and 3 consists of seribantumab 3,000 mg 1-h intravenously (IV) once weekly, until patients meet one or more protocol-specific treatment discontinuation criteria. Dose modifications and/or treatment interruptions to manage treatment related toxicities are permitted during weekly dosing.

For all consented patients assigned to one of the treatment cohorts after eligibility confirmation, treatment starts within 7 days following cohort assignment. Patients are expected to be treated until progressive disease or unacceptable toxicity. Tumor assessments are measured and recorded by the local radiologist beginning at weeks 6 (C2W2), 12 (C3W4), 18 (C5W2) and 24 (C7W2) (+/−2 weeks) and subsequently every 8 weeks (+/−2 weeks) through Year 1, followed by every 12 weeks (+/−2 weeks) thereafter until disease progression and evaluated using the RECIST guidelines (version 1.1). All patients that discontinue treatment for reasons other than disease progression (PD) have a scan performed at the time of the End of Treatment visit. In addition, an independent central review of scans is conducted. All images are submitted to a central imaging facility for this purpose and are assessed by independent reviewers. After patients discontinue seribantumab treatment, survival information and information about subsequent therapies is collected until death or study closure, whichever occurs first. An optional biopsy can be obtained at the time of progression to explore mechanisms of seribantumab resistance.

3. Study Population

The target population for this study is NRG1 gene fusion positive patients with locally advanced or metastatic solid tumors. Such patients have progressed after standard or curative therapy for their tumor type.

A. Inclusion Criteria

To be eligible for participation in the study, patients must meet the following criteria.

i. Locally-advanced or metastatic solid tumor with an NRG1 gene fusion identified through molecular assays, such as PCR, NGS (RNA or DNA) or FISH, by a CLIA certified or similarly accredited laboratory;

ii. Availability of fresh or archived FFPE tumor sample for submission to a central laboratory for post-enrollment confirmation of NRG1 gene fusion status (Cohort 1 only; not a requirement for Cohorts 2 and 3);

iii. Patients have received and progressed after a minimum of one prior standard therapy appropriate for their tumor type and stage of disease, with no further available curative therapy options;

iv. ≥18 years of age

v. Eastern Cooperative Oncology Group (ECOG) performance status (PS) 0, 1 or 2;

vi. Patients must have at least one measurable extra-cranial lesion as defined by RECIST v1.1;

vii. Adequate hepatic function defined as: Serum AST and serum ALT <2.5× upper limit of normal (ULN), or AST and ALT <5×ULN if liver function abnormalities due to underlying malignancy and total bilirubin <2.0×ULN. Subjects with a known history of Gilberts Disease and an isolated elevation of indirect bilirubin are eligible. Subjects with documented hepatic involvement are eligible if total bilirubin ≤3.0×ULN;

viii. Adequate hematologic status, defined as: absolute neutrophil count (ANC) ≥1.5×10⁹/L not requiring growth factor support for at least 7 days prior to Screening, a platelet count ≥100.0×10⁹/L not requiring transfusion support for at least 7 days prior to Screening, and hemoglobin ≥8 g/dL, not requiring transfusion support for at least 7 days prior to screening;

ix. Able to provide informed consent or have a legal representative able and willing to do so;

x. Ability to comply with outpatient treatment, laboratory monitoring, and required clinic visits for the duration of study participation; and

xi. Willingness of men and women of reproductive potential to observe conventional and effective birth control for the duration of treatment and for 3 months following study completion.

B. Exclusion Criteria

Patients who meet any of the following criteria are excluded from participation in this study:

i. Known, actionable oncogenic driver mutation other than NRG1 fusion where available standard therapy is indicated (Cohorts 1 and 2 only; not a requirement for Cohort 3);

ii. Life expectancy <3 months;

iii. Pregnant or lactating;

iv. Prior treatment with ERBB3/HER3 directed therapy (Cohort 1 only);

v. Prior treatment with pan-ERBB or any ERBB/HER2/HER3 directed therapy (Cohort 1 only);

vi. Symptomatic or untreated brain metastases. Patients with asymptomatic brain metastases treated with radiation or surgery and without evidence of progression by imaging at screening are eligible to participate in the study, including those on a stable (e.g., same dose for ≥2 weeks) low-dose corticosteroid regimen;

vii. Received other systemic anticancer therapy (investigational or standard chemotherapy, immunotherapy, or targeted therapy) within 28 days prior to planned start of seribantumab or 5 half-lives, whichever is shorter;

viii. Prior to initiation of seribantumab treatment, patients must have recovered from clinically significant toxicities from prior anticancer or investigational therapy or acute radiation toxicities;

ix. Any other active malignancy requiring systemic therapy;

x. Known hypersensitivity to any of the components of seribantumab or previous “common terminology criteria for adverse events” (CTCAE) grade 3 or higher hypersensitivity reactions to fully human monoclonal antibodies;

xi. Clinically significant cardiac disease, including symptomatic congestive heart failure, unstable angina, acute myocardial infarction within 12 months of planned first dose, or unstable cardiac arrhythmia requiring therapy (including torsades de pointes);

xii. Active uncontrolled systemic bacterial, viral, or fungal infection; and

xiii. Patients who are not appropriate candidates for participation in this clinical study for any other reason as deemed by the investigator.

C. Archival or Fresh Tumor Specimen Requirements

This study only enrolls patients with NRG1 gene fusion positive tumors. A patient's tumor NRG1 status is identified by a molecular assay such as PCR, NGS (RNA or DNA) or FISH as routinely performed by a CLIA certified or other similarly accredited laboratory. For patients who do not have adequate archived tumor tissue available for testing, a fresh tumor biopsy is obtained in advance of study participation, if it can be safely performed. The local NRG1 fusion testing methodologies, which may vary for qualified sites, are performed by CLIA-certified or similarly-accredited laboratories.

In addition, an adequate archival or fresh tumor sample is also required for NRG1 gene fusion confirmation using a qualified RNA-based NGS test performed by a central laboratory, following enrollment and assignment to Cohort 1. Archival and/or fresh tumor samples are collected from Cohort 2 and Cohort 3 patients if available.

D. Study Treatment Discontinuation

A patient can withdraw from the study at any time and for any reason. Over the course of the study, a patient can withdraw from treatment for any of the following reasons:

i. Disease progression (as assessed using RECIST v1.1); with the exception of patients who are deriving clinical benefit, who may be allowed to continue treatment with seribantumab;

ii. Clinically significant drug-related toxicity requiring a recovery period of longer than 3 weeks, unless there is compelling, objective radiological evidence of response, no alternative treatment, and continuation of seribantumab is in the best interest of the patient, and the patient agrees;

iii. Intercurrent illness compromising the ability to fulfill protocol requirements;

iv. Requirement for alternative treatment;

v. Significant non-compliance to protocol;

vi. Withdrawal of consent by the patient;

vii. Patient is lost to follow-up; and

viii. Death.

When a patient is discontinued from treatment for any reason, they are to undergo the assessments in the End of Treatment visit within 4 weeks of the last dose. All patients who discontinue treatment as a result of an adverse event are followed until resolution or stabilization of the adverse event. At the time a patient withdraws from study treatment, an attempt is made to determine the reason(s) for discontinuation.

Upon withdrawal from treatment, the patient continues to be followed for survival and subsequent disease and treatment information every 3 months after completion of the End of Treatment visit.

4. Study Treatment

If a patient is determined to be NRG1 gene fusion positive based upon local testing, investigators determine if the patient meets all other eligibility criteria. A copy of the redacted molecular pathology report, identifying the NRG1 gene fusion per local testing used for eligibility, is submitted for review prior to patient enrollment. Once all study enrollment criteria have been fulfilled, patients are assigned to the appropriate treatment cohort, based upon prior ERBB treatment history and NRG1 fusion testing results. Following enrollment, investigators and/or site staff submit the required tumor samples to a central laboratory for confirmation of NRG1 fusion status per the laboratory manual.

A. Seribantumab

Seribantumab is supplied for IV administration as a sterile, colorless liquid at 25 mg/mL. It is packaged in sterile, single-use, clear borosilicate Type 1 glass vials that are closed with a coated rubber stopper and flip-off cap with flange.

Multiple vials of seribantumab are packaged in a cardboard container. The individual vials, as well as the outside of the cardboard container, are labeled in accordance with regulatory requirements and in compliance with country-specific guidelines.

Seribantumab drug product is stored refrigerated (2-8° C.) with protection from light. Light protection is not required during preparation or infusion. Seribantumab must not be frozen.

Based on available stability data, the concentrate for solution for injection is stable for at least 36 months when stored according to conditions specified in the clinical supply label. Continued stability data are being generated, and longer stability may be available during the course of the study. The date of expiration is noted on the drug label, or via other pharmacy notifications as required by local regulation. Seribantumab is not used beyond the date of expiration. Administration of seribantumab requires multiple vials, all of which should originate from the same lot number. Seribantumab is brought to room temperature prior to mixing with 0.9% normal saline. Vials are not shaken. The appropriate quantity of study drug is removed from the vial and further diluted with 0.9% normal saline to a final total volume of 250 mL and administered over 60 minutes (±15 minutes) using a low protein binding 0.20 or 0.22 micron in-line filter. All infusions are administered over 60 minutes (±15 minutes) in the absence of infusion related reactions. The line is flushed before and after the study drug infusion. Study drug is not administered as a bolus or a push. Seribantumab is administered no less than 7 days after the previous dose.

B. Seribantumab Dosing

Patients enrolled initiate treatment with seribantumab 3,000 mg 1-h IV once weekly until patients meet one or more protocol-specific treatment discontinuation criteria. Dose modifications and/or treatment interruptions to manage treatment related toxicities are permitted during weekly dosing.

C. Management of Toxicity Related to Seribantumab

Patients are monitored during weekly dosing for a period of 28 days (i.e., throughout C1W1, C1W2, C1W3, and C1W4) for the occurrence of DLTs. Any Grade 3 or 4 hematologic or non-hematologic toxicity considered related to seribantumab are considered dose limiting. A DLT is defined as any adverse event (AE) meeting the criteria listed below, occurring during weekly treatment with seribantumab, where the relationship to seribantumab cannot be ruled out. The grading of AEs is based on the “common terminology criteria for adverse events” (CTCAE) version 5.0. Hematologic toxicity includes febrile neutropenia, neutropenic infection, grade 4 neutropenia >7 days, grade ≥3 thrombocytopenia for >7 days, grade ≥3 thrombocytopenia with clinically significant bleeding, grade 4 thrombocytopenia, and grade ≥3 anemia >7 days. Non-hematologic toxicity includes (1) grade ≥3 nausea, vomiting, or diarrhea lasting more than 72 hours despite optimal medical support with anti-emetics or anti-diarrheals, (2) grade 4 (life-threatening) vomiting, or diarrhea are considered DLTs irrespective of duration, (3) any other grade ≥3 AE, except Grade ≥3 fatigue and anorexia lasting for <7 days or Grade ≤2 infusion related reactions (for grade 3 or higher infusion-related reactions (IRRs), seribantumab is permanently discontinued)

Any toxicity, regardless of CTCAE grade, resulting in discontinuation or dose reduction of seribantumab treatment, with the exception of symptoms related to disease progression [PD], is considered a DLT.

D. Management of Infusion Related Reactions

Like other IV infusions of monoclonal antibodies, seribantumab administration may be associated with infusion related reactions (IRRs). Infusion related reactions are defined according to the National Cancer Institute CTCAE (Version 5.0) definition of an allergic reaction/infusion reaction and anaphylaxis. In past clinical studies (n=847 patients treated), IRRs with seribantumab have been rare with <1% of patients experiencing an IRR, of which all were Grade 1 or 2. Study site policies or the following treatment guidelines set forth in Table 2 are used for the management of infusion reactions.

TABLE 2
Treatment Guidelines

Grade 1
Slow infusion rate by 50%
Monitor patient every 15 minutes for worsening of condition
Grade 2
Stop infusion
Administer diphenhydramine hydrochloride 50 mg IV, acetaminophen
500-650 mg orally, and oxygen
Resume infusion at 50% of the prior rate once infusion reaction has
resolved
Monitor patient every 15 minutes for worsening of condition
For all subsequent infusions, pre-medicate with dexamethasone 10 mg
orally or IV
Grade 3
Stop infusion and disconnect infusion tubing from patient
Administer diphenhydramine hydrochloride 50 mg IV, dexamethasone
10 mg IV, bronchodilators for bronchospasm, and other medications
or oxygen as medically necessary
No further treatment with seribantumab is permitted
Grade 4
Stop the infusion and disconnect infusion tubing from patient
Administer epinephrine, bronchodilators or oxygen as indicated for
bronchospasm
Administer diphenhydramine hydrochloride 50 mg IV, dexamethasone
10 mg IV
Consider hospital admission for observation
No further treatment with seribantumab is permitted

For patients who experience a Grade 1 or Grade 2 infusion reaction, future infusions can be administered over 90 minutes. In addition, for patients who experience a subsequent Grade 1 or 2 infusion reaction, administer dexamethasone 10 mg IV. All subsequent infusions are pre-medicated with diphenhydramine hydrochloride 50 mg IV, dexamethasone 10 mg IV, and acetaminophen 500-650 mg orally.

For patients who experience a Grade 3 or 4 infusion reaction, an anti-seribantumab antibody titer is taken as close to the onset of the infusion reaction as possible. An anti-seribantumab antibody titer is also obtained at the resolution of the event and 28 days (+/−2 days) following the event.

E. Toxicity Management Guidelines

When a patient experiences any Grade 3 or Grade 4 hematologic or non-hematologic toxicity, excluding infusion related reactions related to seribantumab, the following toxicity management guidelines are followed:

Dose Interruptions and Reductions for Hematologic Toxicity: for ≥Grade 3 hematologic toxicity, seribantumab dosing is held until resolving to ≤Grade 2 or the patient's baseline. Once the hematologic toxicity resolves to ≤Grade 2 or the patient's baseline, seribantumab is restarted at a 25% reduction of the original dose.

For recurrence of a ≥Grade 3 hematologic toxicity, seribantumab is held again until resolving to ≤Grade 2 or the patient's baseline. Once the hematologic toxicity resolves to ≤Grade 2 or the patient's baseline, seribantumab is restarted at a 50% reduction of the original dose.

For patients who have had dose reductions of seribantumab due to hematologic toxicity, investigators can restart seribantumab at the original assigned dose, provided the toxicity has resolved to ≤Grade 1 on the reduced dose for at least one cycle of treatment.

Seribantumab is permanently discontinued if the patient experiences a recurrent grade 3 or higher treatment related hematologic toxicity, despite a 50% dose reduction.

Dose Interruptions and Reductions for Non-Hematologic Toxicity: for ≥Grade 3 non-hematologic toxicity, seribantumab dosing is held until resolving to ≤Grade 1 or the patient's baseline. Once the non-hematologic toxicity resolves to ≤Grade 1 or the patient's baseline, seribantumab is restarted at a 25% reduction of the original dose.

For recurrence of a ≥Grade 3 non-hematologic toxicity, seribantumab is held again until resolving to ≤Grade 1 or the patient's baseline. Once the non-hematologic toxicity resolves to ≤Grade 1 or the patient's baseline, seribantumab is restarted at a 50% reduction of the original dose.

For patients who have had dose reductions of seribantumab due to non-hematologic toxicity, seribantumab can be restarted at the original assigned dose, provided the toxicity has resolved to ≤Grade 1 on the reduced dose for at least one cycle of treatment.

Seribantumab is permanently discontinued if the patient experiences a recurrent grade 3 or higher treatment related non-hematologic toxicity, despite a 50% dose reduction.

Dose re-escalation is not permitted for patients who experience (1) recurrent grade 3 adverse events determined to be clinically significant despite dose-reduction or (2) recurrent grade 4 adverse events despite dose-reduction.

Seribantumab is permanently discontinued for patients who experience life-threatening grade 4 adverse events.

F. Potential Toxicities with Seribantumab

In a previously conducted Phase I dose escalation and expansion study evaluating seribantumab as a monotherapy in 43 advanced solid tumor patients refractory to standard therapy, the most commonly reported adverse events included Grade 1 or 2 rash, nausea, diarrhea and fatigue. Grade 1 hypomagnesemia was also observed. Overall, a maximum tolerated dose was not reached in this study and there were no dose limiting toxicities observed in the 22 patients treated at the highest dose level studied, during the dose escalation and dose expansion phases of the study. The highest dose level studied, which was the 40/20 weekly dosing regimen (40 mg/kg loading dose followed by 20 mg/kg once weekly), was identified as the recommended dosing regimen for subsequently conducted phase 1 and 2 studies, in which seribantumab was combined with standard chemotherapy, hormonal or targeted therapies.

Adverse events in this seribantumab monotherapy study, involving a total of 43 patients with advanced solid tumors, were initially defined as any treatment-emergent adverse event (TEAE) having >20% incidence (all grades and regardless of relationship). Based upon this definition, the following adverse events were observed, with the majority being mild to moderate in severity: fatigue, nausea, diarrhea, vomiting, decreased appetite, hyperglycemia, hypokalemia, and rash.

Overall, no dose limiting toxicities related to seribantumab were observed in the 22 patients treated with the 40/20 mg/kg weekly dosing regimen level in this monotherapy study. In the current study, 6 patients were treated in a safety run-in phase with an induction regimen consisting of a 3,000 mg loading dose followed by 2,000 mg weekly for 3 weeks. No patients in the safety run-in cohort experienced a dose limiting toxicity and TEAEs primarily consisted of Grade 1 diarrhea, nausea, fatigue, rash and pruritis. Enrollment continued under Induction Regimen 2 consisting of 3,000 mg once weekly for 4 weeks. The next 6 patients enrolled were treated with Induction Regimen 2. Overall, none of the first 6 patients treated with Induction Regimen 2 experienced a DLT, and TEAEs primarily consisted of Grade 1 diarrhea, nausea, fatigue, rash and pruritis.

G. Dose Modifications

Patients who experience a clinically significant adverse event (Grade ≥3), can have seribantumab dosing held for up to 3 weeks to allow for recovery and, upon restarting, have the seribantumab dose reduced by either 25% (first occurrence) or 50% (recurrence), as follows:

TABLE 3
Dose Modification - Weekly Dosing

| | |
|---|---|
| Original dose level | 3,000 mg |
| 25% dose reduction | 2,250 mg |
| 50% dose reduction | 1,500 mg |

Seribantumab is permanently discontinued for patients who experience life-threatening Grade 4 adverse events. Seribantumab is permanently discontinued for patients who experience clinically significant, drug-related adverse events requiring a recovery period of longer than 3 weeks, unless there is compelling, objective radiological evidence of response and no alternative treatment.

H. Clinical Procedures and Assessments

All clinical procedures are performed in accordance with the schedule of assessments set forth in Table 4.

TABLE 4
Schedule of Assessments

| | Evaluation/ Procedure | | | |
|---|---|---|---|---|
| | | | End of | |
| | | | Treatment | Survival Follow-Up |
| | Screening | | Visit | Every 3 months |
| | (D-28 | Treatment ¹⁶ | (EOT)¹³ | after EOT¹⁵ |
| Visit Window | to 0) | ±2 days | ±3 days | ±1 month |
| Informed consent | X | | | |
| Medical, surgical, | X | | | |
| malignancy history | | | | |
| Archived tumor | X | | X¹⁴ | |
| tissue or fresh | | | | |
| biopsy | | | | |
| Urine or serum pregnancy test | X | Every 28 days | X | |
| Physical Exam and | X | X | X | |
| ECOG PS¹ | | | | |
| Vital signs | X⁸ | | X | |
| CBC | X^{2, 8} | Weekly through C1, then every 14 days starting | X | |
| Serum chemistry | X^{3, 8} | C2W1 then every 28 days starting after 1 year on | X | |
| | | treatment | | |
| Blood sample(s) | | C1W1, C1W3, C2W3, C3W3, | | |
| for PK⁴ | | C4W1, C5W1, and C6W1 | | |
| 12-lead ECG | X⁵ | | X⁵ | |
| Serum for Ig⁶ | | C1W1, C1W3, C2W1, C2W3, C3W1 and C3W3, | X⁶ | |
| | | then every 28 days starting at C4W1 | | |
| Whole blood for | | C1W1 and C2W1 | X | |
| cfDNA⁷ | | | | |
| Concomitant | X | X | X | |
| meds⁸ | | | | |
| Seribantumab | | | | |
| dosing⁹ | | | | |
| AE/SAE | X | | | |
| assessment and | | | | |
| reporting¹⁰ | | | | |
| Disease evaluation | X¹¹ | Week 6, Week 12, Week 18, Week 24; | X¹² | |
| | | then every 8 weeks (Year 1); | | |
| | | then every 12 weeks | | |

| | |
|---|---|
| Overall Survival | X |
| Reporting¹⁵ | |

¹Review of systems: HEENT, extremities, GI/abdomen, lymph nodes, musculoskeletal, respiratory/pulmonary, and skin, body weight, and height during Screening. Symptom-directed physical and neurological examinations, including measurement of weight, are performed at other time points. After screening, physical exams occur weekly during Cycle 1 and at Week 1 of every cycle thereafter.
²Hemoglobin, hematocrit, RBC count, WBC count with differential (neutrophils [count and percent] and lymphocytes, monocytes, eosinophils, basophils [percent], and platelet count. Ad hoc samples may be requested.
³Serum or plasma chemistries (non-fasting), including alkaline phosphatase, albumin, ALT, AST, blood urea nitrogen (BUN), cholesterol, creatinine, glucose, LDH, uric acid, total and direct bilirubin, total protein, sodium, potassium, calcium, chloride, bicarbonate, magnesium and phosphate. Ad hoc samples may be requested.
⁴Seribantumab PK sampling: At C1W1 sampling occurs immediately prior to dosing, at the end of infusion (EOI) and 1 hour after EOI. At C1W3, C2W1, C2W3, C3W1, C3W3, C4W1, C5W1, and C6W1: Samples are collected immediately prior to each dose and at the End of infusion (EOI). Sampling occurs within 15 minutes of starting or completing the seribantumab infusion. Ad hoc PK samples can be requested.
⁵Screening ECG is performed in triplicate. EOT ECG reading is repeated only if the initial reading at the EOT visit showed treatment emergent abnormalities. Ad hoc ECGs can be requested.
⁶Immunogenicity samples are collected prior to dosing at scheduled time points. If a patient experiences an infusion reaction on study, an anti-seribantumab antibody assay is taken within 24 hours of the event. For patients who experience a grade 3 or 4 infusion reaction, an anti-seribantumab antibody titer is taken as close to the onset of the infusion reaction as possible, upon resolution and 28 days (±2 days) following the event.
⁷Whole blood for cfDNA analysis is obtained prior to treatment on C1W1 and C2W1. Whole blood for cfDNA analysis is obtained at the EOT visit even if radiographic disease assessment is not performed.
⁸Performed prior to seribantumab administration.
⁹Seribantumab dosing consists of weekly 1-h infusions until study treatment discontinuation criteria are met. Seribantumab doses are administered no less than 7 days apart.
¹⁰All AEs and SAEs are collected and reported from the time of informed consent through the EOT visit.
¹¹Baseline Disease Assessment: Radiographic tumor measurements using CT (computerized tomography) or CT/PET (positron emission tomography) or magnetic resonance imaging (MRI) of the chest, abdomen, and pelvis, with additional regions affected by disease as appropriate, and CT or MRI of brain (if brain involvement is suspected) within 28 days of seribantumab dosing (first dose). Contrast should be utilized (excluding CT of the chest) unless there is a clear contraindication (e.g., decreased renal function or allergy that cannot be addressed with standard prophylactic treatments). Disease assessments utilize RECIST v1.1. Disease assessments occur prior to seribantumab administration on dosing days and occur within a ±14-day window.
¹²All patients that come off treatment for reasons other than progressive disease have a disease assessment performed at the EOT visit.
¹³End of Treatment (EOT) visit is completed within 4 weeks of the last dose of study drug administration.
¹⁴An optional fresh tumor biopsy is performed at the time of progression and prior to the completion of the EOT assessments if feasible to evaluate potential patterns of resistance to seribantumab.
¹⁵Every survival follow-up should include collection of any new anti-cancer therapies and procedures taken after EOT visit. Should patients refuse or drop out of survival follow-up, attempts should be made to obtain any death information available via public records.
¹⁶ If the Investigator adjusts to once weekly dosing after a period of Q2W dosing, procedures should be performed according to the schedule of assessments for weekly dosing.

I. Concomitant and Prohibited Therapies

Standard supportive medications can be used in accordance with institutional guidelines and Investigator discretion. These can include hematopoietic growth factors to treat neutropenia, thrombocytopenia or anemia in accordance with American Society for Clinical Oncology (ASCO) Guidelines (but not for prophylaxis in Cycle 1), transfusions, anti-emetics, anti-diarrheals, antibiotics, antipyretics, and corticosteroids (up to 10 mg per day prednisone or equivalent, unless a compelling clinical rationale for a higher dose is articulated; permitted corticosteroid uses include topical/cutaneous, ophthalmic, nasal and inhalational steroids, as well as short courses to treat asthma, chronic obstructive pulmonary disease, or other non-cancer related conditions).

Concomitant therapy (non-investigational products) includes any prescription medication, over-the-counter preparation, herbal therapy, or radiotherapy used by a patient between the 28 days preceding study treatment initiation and the study treatment discontinuation visit. After the End of Treatment Visit, only anti-cancer therapies are collected in addition to survival information.

The following therapies are not permitted while on study treatment: (1) other anti-neoplastic therapy, including cytotoxics, targeted agents, endocrine therapy or other antibodies (patients who have been on GnRH analogues for more than 90 days prior to study entry may continue while on study), (2) radiotherapy (patients who require a short course of palliative radiotherapy may continue on the study treatment after discussion between the investigator and sponsor), and (3) any other investigational therapy.

J. Adverse Event and Hospitalization Assessment Reporting

Investigators complete all routine and standard of care assessments to evaluate for toxicity and symptoms of drug-induced adverse events. This can include, but is not limited to, verbal reports from the patient and/or caregiver, physical examination and laboratory findings. Adverse events are collected and reported throughout the course of the study.

Vital signs are collected at Screening and prior to seribantumab administration at dosing visits and include height (screening only), weight, resting blood pressure, pulse, respiratory rate, and temperature.

The Eastern Cooperative Oncology Group (ECOG) performance status (PS) is obtained by questioning the patient about their functional capabilities.

A 12-lead electrocardiogram (ECG) includes a description of the cardiac rate, rhythm, interval durations, and an overall impression. The corrected QT interval (QTc) is calculated using the Fridericia method (QTcF).

Tumor response is evaluated by the local radiologist according to RECIST version 1.1 to establish disease progression by CT or MRI. In addition, other radiographic or scintigraphic procedures (such as radionuclide bone scans), as deemed appropriate by the investigator, are performed to assess sites of neoplastic involvement. The same method of assessment is used throughout the study. Independent retrospective central reviews of all scans can be conducted in addition to review performed by the local radiologist. Investigators choose target and non-target lesions in accordance with RECIST v1.1 guidelines. Follow-up measurements and overall response is also in accordance with these guidelines. To be assigned a status of confirmed partial response (PR) or complete response (CR), changes in tumor measurements must be confirmed by repeated assessments that are performed ≥30 days after the criteria for response are first met.

Disease is assessed every 6 weeks (±2 weeks) from treatment assignment through week 24, then every 8 weeks (±2 weeks) through the end of Year 1, followed by every 12 weeks (±2 weeks) until the patient is removed from study directed treatment and completes the End of Treatment visit. All patients that come off treatment for reasons other than progressive disease have a scan at the time of the End of Treatment visit.

K. Laboratory Procedures and Assessments

The complete blood count (CBC) includes the following: hemoglobin, hematocrit, platelet count, RBC, WBC with differential (neutrophils, lymphocytes, monocytes, eosinophils, basophils and other cells).

Serum chemistry (non-fasting) includes electrolytes (sodium, potassium, calcium, chloride, bicarbonate, magnesium and phosphate), BUN, serum creatinine, cholesterol, glucose, total and direct bilirubin, AST, ALT, alkaline phosphatase, LDH, uric acid, total protein and albumin.

A urine or serum pregnancy test is obtained during Screening, every 28 days, and the End of Treatment visit for all females of childbearing potential. Exempt female patients include those who have undergone a bilateral oophorectomy or hysterectomy, or those who are menopausal (defined as absence of a menstrual cycle for at least 12 consecutive months).

Plasma samples for pharmacokinetic (PK) samples are obtained from patients. At C1W1 sampling occurs immediately prior to dosing, at the end of infusion (EOI) and 1 hour after EOI. At C1W3, C2W1, C2W3, C3W1, C3W3, C4W1, C5W1, and C6W1: Samples are collected immediately prior to each dose and at the End of infusion (EOI).

Serum samples are collected prior to dosing at the scheduled time points to determine the presence of an immunologic reaction to seribantumab (i.e., human anti-human antibodies; HAHA) and for any patients who experience a Grade 3 or higher infusion reaction during seribantumab administration. A laboratory manual is provided with instructions for collecting, processing, and shipping these samples.

Biomarker data is explored from collected tissue (prior to treatment and at the EOT visit for those patients who undergo an optional biopsy) and whole blood samples for cell free DNA (cfDNA) to assess potential associations with tumor response. Efficacy outcomes considered for pre-specified mechanistic biomarker analysis include, but not be limited to OS, PFS, and ORR.

If archived tumor tissue is available, it is submitted in lieu of obtaining a fresh tumor biopsy, for central confirmation of NRG1 gene fusion status at the time of study enrollment for patients assigned to Cohort 1. For patients where adequate archival tissue is not available, tumor biopsy procedures performed on an outpatient basis and associated with a low risk of major complications, per the recent guidance from ASCO (Levit et al., J Clin Oncol. 2019; 37:2368-2377), can be considered by the treating physician, in accordance with site specific consent and standard procedures.

For Cohort 2 patients, in cases where recent progression on HER pathway therapy (e.g., afatinib, HER2-based treatment, MCLA128) has been documented or observed, a fresh tumor biopsy that can be performed on an outpatient basis and that is associated with a low risk of major complications per ASCO guidance is preferred in addition to available archival tissue in order to better understand the mechanism for progression on prior treatment.

Central confirmation of NRG1 gene fusion status is performed on a prospective basis by Caris Life Sciences, utilizing their RNA-based NGS test, MI Transcriptome™. Immediately following enrollment, Investigators and study staff are required to obtain, process and ship the required archival tumor tissue for central confirmatory testing. A minimum of 55 patients, with centrally confirmed NRG1 gene fusion positive tumors, based on an RNA-based NGS testing method, are enrolled into Cohort 1. Central confirmatory testing for patients initially enrolled and assigned to Cohort 2 or Cohort 3 is not required.

If central laboratory testing fails to confirm the presence of an NRG1 fusion, or insufficient tumor tissue is provided for central testing after enrollment, patients are permitted to continue participating in the study at the Investigator's discretion, as long as they do not meet criteria for treatment discontinuation.

Whole blood samples are collected prior to dosing at specified time points. The samples are used to conduct exploratory studies to further characterize and correlate possible biomarkers that may help to predict or evaluate response to seribantumab in NRG1 fusion positive advanced solid tumor patients. In the event that there is remaining sample available after conducting these analyses, it is used for future analysis of biomarkers that may be mechanistically linked to seribantumab activity.

5. Adverse Events and Reporting

An adverse event (AE) is any untoward medical occurrence in a patient or clinical investigation patient administered a pharmaceutical product and does not necessarily have to have a causal relationship with this treatment. An adverse event can therefore be any unfavorable and unintended sign, including abnormal laboratory findings, symptoms, or diseases temporally associated with the use of a medicinal product, whether or not considered related to the medicinal product.

All adverse events, complaints, or symptoms that occur from the time that written informed consent has been obtained through the end of treatment visit are recorded (events that occur prior to first dose of study drug are considered medical history, events that occur on or after the first dose of study drug are considered treatment emergent adverse events). Documentation is supported by an entry in the patient's source medical records. Clinically significant abnormal laboratory or other examination (e.g., ECG) findings that are detected during the study, or are present at Screening and significantly worsen during the study, are reported as adverse events. Each adverse event is evaluated for duration, severity, and causal relationship with the investigational product, underlying disease or other factors.

Worsening of a pre-existing medical condition, (i.e., diabetes, migraine headaches) is considered an adverse event if there is either an increase in severity, frequency, or duration of the condition or an association with significantly worse outcomes. Disease progression in and of itself is not considered an adverse event or serious adverse event.

Interventions for pretreatment conditions (i.e., elective cosmetic surgery) or medical procedures that were planned prior to study enrollment are not considered adverse events.

For an adverse event leading to death, the outcome is recorded with the event causing death. Adverse events that are ongoing at the end of study or time of death are to be noted as “continuing”.

The Investigator exercises his or her medical and scientific judgment in deciding whether an abnormal laboratory finding or other abnormal assessment is clinically significant. Clinically significant abnormal laboratory values occurring during the clinical study are followed until repeat tests return to normal, stabilize, or are no longer clinically significant. Any abnormal test that is determined to be an error does not require reporting as an adverse event.

Each adverse event is graded according to the National Cancer Institute (NCI) “common terminology criteria for adverse events” (CTCAE) version 5.0. For events not listed in the CTCAE, severity is designated as mild, moderate, severe, or life-threatening, or fatal which correspond to Grades 1, 2, 3, 4, and 5, respectively on the NCI CTCAE, with the following definitions as set forth in Table 5:

TABLE 5
Definitions

Mild/Grade 1:
An event not resulting in disability or incapacity and which resolves
without intervention.
Moderate/Grade 2:
An event not resulting in disability or incapacity, but which requires
intervention.
Severe/Grade 3:
An event resulting in temporary disability or incapacity and which
requires intervention.
Life-threatening/Grade 4:
An event in which the patient was at risk of death at the time of the
event.
Fatal/Grade 5:
An event that results in the death of the patient.

A serious adverse event (SAE) is any untoward medical occurrence that at any dose: (1) results in death, (2) is life-threatening, (3) requires in-patient hospitalization or prolongation of existing hospitalization, (4) results in persistent or significant disability/incapacity, (5) is a congenital anomaly or birth defect, or (6) is an important medical event.

While the term “severe” is often used to describe the intensity (severity) of an event, the event itself may be of relatively minor significance (such as a severe headache). This is not the same as “serious”, which is based on a patient/event outcome or action criteria usually associated with events that pose a risk to a patient's life or functioning.

6. Statistical Methods

A. Endpoints

The primary endpoint is objective response rate as assessed by independent radiologic review according to RECIST 1.1. Secondary endpoints include Duration of Response (DoR), safety of seribantumab in NRG1 gene fusion positive patients, Progression-free Survival (PFS), Overall Survival (OS), and Clinical Benefit Rate (CR, PR, SD>24 weeks). Exploratory endpoints include pharmacokinetic parameters following weekly, Q2W and Q3W dosing and exploring the association between mechanistically linked biomarkers and clinical outcomes.

B. Analysis Populations

The safety population is used primarily for the analysis of safety data and consists of all enrolled patients who receive 1 or more doses of seribantumab.

The Intent to Treat Population (ITT) includes all eligible centrally confirmed NRG1 gene fusion patients assigned and enrolled to Cohort 1 who receive at least one dose of seribantumab therapy with the 12-Week Target Induction Regimen or the weekly dosing regimen.

C. Determination of Sample Size

The trial is designed to provide statistically persuasive evidence of a clinically meaningful effect of seribantumab if the lower boundary of a 2-sided 95% exact binomial confidence interval (CI) about the estimated ORR exceeds a minimal threshold of 30%. This threshold for level of evidence for benefit would be consistent with the standard used for approved targeted therapies for genomically defined populations of patients who stop responding to previous standard therapies. Under the planned primary efficacy analysis, if the observed ORR is ≥50% when seribantumab is administered to patients with NRG1 gene fusion positive cancers, then a trial with a sample size of 55 patients with central confirmation of NRG1 gene fusion status, who have been enrolled and treated with either the 12-Week Target Induction Regimen and transitioned without interruption to weekly dosing, or once weekly dosing from the time of enrollment, would have more than 80% power to achieve the pre-specified targeted threshold for positivity.

D. Statistical Considerations

Categorical variables are summarized by frequency distributions (number and percentages of patients) and continuous variables are summarized by descriptive statistics (mean, standard deviation, median, minimum, maximum).

Disposition of patients is summarized, including those screened, treated, and discontinued. Reason for discontinuation is summarized. Demographic and baseline characteristics are summarized. Medical history and prior medications are tabulated.

Due to the ongoing COVID-19 pandemic, additional analyses (e.g., sensitivity analysis) are performed to assess the impact of COVID-19 on clinical trial data. Documentation for reasons for failing to obtain a protocol specified assessment and/or alternative procedures used to collect safety and efficacy data is required.

Objective response rate (ORR) is determined by RECIST v1.1 (CR+PR) and is assessed by independent radiographic review. An estimate of the ORR and its 95% CI is calculated. The primary efficacy analysis is performed on the ITT population for Cohort 1. Predefined factors for response assessment include: (1) The number of prior systemic treatments for locally advanced and/or metastatic disease (1, 2 or 3); (2) Tumor type; and (3) NRG1 gene fusion partner.

Duration of response is based on independent radiographic review. DOR is calculated for subjects who achieve a confirmed CR or PR. For such subjects, DOR is defined as the number of months from the start date of CR or PR (whichever response status is observed first and subsequently confirmed), to the date of first documented radiographical progression of disease using RECIST v1.1, or death from any cause, whichever comes first. The duration of response is determined at the time of first radiographic progression for all patients, including those patients who undergo weekly re-induction dosing with seribantumab.

Progression-free Survival (PFS) is based on investigator assessment. PFS is defined as the time from the date of seribantumab treatment initiation (Dose 1) to the first documented radiographical progression of disease using RECIST 1.1, or death from any cause, whichever comes first. The Kaplan-Meier method is used to estimate PFS for each treatment cohort. In addition, an analysis of PFS following initiation of seribantumab is compared to the PFS observed for each patient during their most recent line of therapy prior to initiating seribantumab. PFS is determined at the time of first radiographic progression for all patients, including those patients who undergo weekly re-induction dosing with seribantumab.

Overall Survival (OS) is defined as the time from the date of treatment seribantumab treatment initiation (Dose 1) to the date of death from any cause. The Kaplan-Meier method is used to estimate OS for each treatment cohort. In addition to estimating the overall distribution for OS, the median, 6-month and 12-month survival rates are estimated.

Additional OS, PFS, ORR and TTP sensitivity analyses are conducted using various study populations. In addition, various censoring rules are applied in the analysis of PFS to assess sensitivity to changes in specifications. These are clearly detailed in the statistical analysis plan (SAP).

Safety analyses (adverse events and laboratory analyses) are performed using the safety population. Adverse events are coded using the latest MedDRA dictionary. Severity is graded according to the NCI CTCAE version 5.0.

Treatment-emergent adverse events (TEAEs), TEAE grade 3 and higher, TEAE-related, SAEs, and discontinuation due to AE are reported by frequency and percent summaries. Adverse events are summarized by System Organ Class and preferred term. All adverse event data are listed by patient. TEAEs are defined as any event that occurred after the first dose of study drug and was not present prior to study drug administration or worsened in severity after study drug administration. TEAEs are collected through the end of treatment visit.

Laboratory, vital signs, and ECG data are summarized according to parameter type.

Biomarker data from collected tissue and serum is used to search for enriched populations having the potential of higher rates of tumor response. Efficacy outcomes considered in these exploratory analyses include ORR, OS, and PFS. Kaplan-Meier methods are used in these descriptive analyses.

Plasma concentrations by subject, cycle, day, and time are obtained and documented at various time points during treatment with seribantumab An independent retrospective central review of scans is conducted to assess ORR (primary endpoint) and Duration of Response (secondary endpoint). All images are submitted to a central imaging facility for this purpose and are assessed by independent reviewers in accordance with the Imaging Charter.

7. Previous CRESTONE Dosing Schedules

Prior to approval for CRESTONE, Protocol Version 4.0 (described in detail above), treatment for all eligible patients consisted of initial Induction (once weekly dosing for 4 weeks) followed by every two weeks (Q2W) maintenance dosing with seribantumab.

A. Induction Regimen 1

Seribantumab 3,000 mg 1-h IV as a loading dose at the C1W1 visit, followed by 2,000 mg 1-h IV once weekly at C1W2, C1W3 and C1W4. There were no DLTs observed with Induction Regimen 1. Upon review, the next 6 patients enrolled and initiated treatment with Induction Regimen 2.

B. Induction Regimen 2

Seribantumab 3,000 mg 1-h IV once weekly at the C1W1, C1W2, C1W3 and C1W4 visits. There were no DLTs observed in the first 6 patients treated with Induction Regimen 2. Upon review, all subsequently enrolled patients treated under Protocol Version 2.0 initiated treatment with Induction Regimen 2.

With approval of Protocol Version 3.0, subsequently enrolled patients initiated treatment with the 12-Week Target Induction Regimen.

C. 12-Week Target Induction Regimen

Seribantumab 3,000 mg 1-h IV once weekly for a total of 12 weeks (C1W1, C1W2, C1W3, C1W4, C2W1, C2W2, C2W3, C2W4, C3W1, C3W2, C3W3, and C3W4 visits). Notably, patients enrolled to Induction Regimen 2, who were continuing weekly induction phase dosing at the time of approval for Protocol Version 3.0, were switched to the extended 12-Week Target Induction Regimen.

D. Q2 Week Dosing:

Prior to approval of Protocol Version 4.0, all patients who completed induction phase dosing with Induction Regimen 1, Induction Regimen 2 or the 12-week Target Induction Regimen, who continued study-directed treatment, subsequently received seribantumab 3,000 mg 1-h IV once every 2 weeks, initiating approximately 14 days after completion of the final weekly induction dosing. For these patients, dosing continued every 2 weeks until patients met one or more protocol-specific treatment discontinuation criteria.

Protocol Version 2.0 included Q2W dosing for 6 doses (consolidation dosing) followed by Q3W dosing (maintenance dosing) for the remainder of study participation. Q3W dosing was removed from the protocol with the approval of Protocol Version 3.0. No patients were treated with Q3W dosing.

E. Re-Induction Dosing:

Prior to approval of Protocol Version 4.0, patients continuing treatment with Q2W dosing, who were (a) demonstrating signs of worsening disease or clinical progression in between radiographic assessments, or (b) experiencing radiographic or clinical progression that has been documented and in the opinion of the investigator the patient may continue to derive benefit, or (c) had experienced a prolonged treatment interruption >3 weeks in the absence of treatment related toxicity (e.g., to manage COVID-19 complications), could be re-induced and switched back to a weekly dosing schedule after consultation. Unless patients required dose modifications during previous weekly dosing, re-induction consisted of seribantumab 3,000 mg 1-h IV weekly.

With the approval of Protocol Version 3.0, patients were required to meet the following criteria to be considered for weekly re-induction dosing: (1) patient informed of purpose, risk, and benefit of, and alternative therapies to re-induction with seribantumab; and patient provided verbal or written consent to re-induction with seribantumab, (2) absence of clinical symptoms or signs indicating clinically significant disease progression, (3) no clinically significant decline in performance status, (4) absence of rapid disease progression or threat to vital organs or critical anatomical sites (e.g., CNS metastasis, respiratory failure due to tumor involvement, spinal cord compression) requiring urgent alternative medical intervention, (5) no significant, unacceptable, or irreversible toxicities related to seribantumab, and (6) if dose reduction was required during previous weekly seribantumab dosing, weekly re-induction dosing is initiated at the final modified dose level that was previously administered.

8. Guidance for Switching Patients to Continuous Weekly Dosing

Upon approval of Protocol Version 4.0, patients enrolled under a previous protocol version are managed as follows. Patients who are actively receiving weekly induction dosing (i.e., Induction Regimen 2 or 12-Week Target Induction Regimen) continue weekly dosing at their current dose level until disease progression or unacceptable toxicity. Patients who have completed weekly induction dosing and are receiving every two week (Q2W) dosing under previous versions of the protocol are switched to weekly dosing provided the following criteria are met: (1) patient informed of purpose, risk, and benefit of, and alternative therapies to continuous weekly dosing with seribantumab; and patient provided verbal or written consent to continuous weekly dosing with seribantumab, (2) absence of clinical symptoms or signs indicating clinically significant disease progression, (3) no clinically significant decline in performance status, (4) absence of rapid disease progression or threat to vital organs or critical anatomical sites (e.g., CNS metastasis, respiratory failure due to tumor involvement, spinal cord compression) requiring urgent alternative medical intervention, (5) no significant, unacceptable, or irreversible toxicities related to seribantumab, and (6) if dose reduction was required during previous weekly seribantumab dosing, weekly dosing is re-initiated at the final modified dose level that was previously administered and tolerated by the patient.

Unless patients required dose modifications during previous weekly dosing, re-initiation of weekly dosing consists of seribantumab 3,000 mg 1-h IV weekly, with proper dose modifications and/or interruptions when a patient experiences any treatment related Grade 3 or Grade 4 hematologic or non-hematologic toxicity.

Those skilled in the art will recognize and are able to ascertain and implement using no more than routine experimentation, many equivalents of the specific embodiments described herein. Such equivalents are intended to be encompassed by the following claims. Any combinations of the embodiments disclosed in the dependent claims are within the scope of the disclosure.

All patents, patent applications and publications cited herein are incorporated herein by reference in their entireties.

SEQUENCE SUMMARY

| SEQ | | SOURCE | | |
|---|---|---|---|---|
| ID | | OR | | |
| NO: | DESIGNATION | FORMAT | TYPE | SEQUENCE |
| 1 | Heavy Chain | Human | DNA | gaggtgcagc tgctggagag cggcggaggg |
| | Variable Region | VH | | ctggtccagc caggcggcag cctgaggctg |
| | (VH) of | | | tcctgcgccg ccagcggctt caccttcagc |
| | seribantumab | | | cactacgtga tggcctgggt gcggcaggcc |
| | | | | ccaggcaagg gcctggaatg ggtgtccagc |
| | | | | atcagcagca gcggcggctg gaccctgtac |
| | | | | gccgacagcg tgaagggcag gttcaccatc |
| | | | | agcagggaca acagcaagaa caccctgtac |
| | | | | ctgcagatga acagcctgag ggccgaggac |
| | | | | accgccgtgt actactgcac caggggcctg |
| | | | | aagatggcca ccatcttcga ctactggggc |
| | | | | cagggcaccc tggtgaccgt gagcagc |
| 2 | Heavy Chain | Human | PROTEIN | Glu Val Gln Leu Leu Glu Ser Gly Gly Gly |
| | Variable Region | VH | | Leu Val Gln Pro Gly Gly Ser Leu Arg Leu |
| | (VH) of | | | Ser Cys Ala Ala Ser Gly Phe Thr Phe Ser |
| | seribantumab | | | His Tyr Val Met Ala Trp Val Arg Gln Ala |
| | | | | Pro Gly Lys Gly Leu Glu Trp Val Ser Ser |
| | | | | Ile Ser Ser Ser Gly Gly Trp Thr Leu Tyr |
| | | | | Ala Asp Ser Val Lys Gly Arg Phe Thr Ile |
| | | | | Ser Arg Asp Asn Ser Lys Asn Thr Leu Tyr |
| | | | | Leu Gln Met Asn Ser Leu Arg Ala Glu Asp |
| | | | | Thr Ala Val Tyr Tyr Cys Thr Arg Gly Leu |
| | | | | Lys Met Ala Thr Ile Phe Asp Tyr Trp Gly |
| | | | | Gln Gly Thr Leu Val Thr Val Ser Ser |
| 3 | Light Chain | Human | DNA | cagtccgccc tgacccagcc cgccagcgtg |
| | Variable Region | VL | | agcggcagcc caggccagag catcaccatc |
| | (VL) of | | | agctgcaccg gcaccagcag cgacgtgggc |
| | seribantumab | | | agctacaacg tggtgtcctg gtatcagcag |
| | | | | caccccggca aggcccccaa gctgatcatc |
| | | | | tacgaggtgt cccagaggcc cagcggcgtg |
| | | | | agcaacaggt tcagcggcag caagagcggc |
| | | | | aacaccgcca gcctgaccat cagcggcctg |
| | | | | cagaccgagg acgaggccga ctactactgc |
| | | | | tgcagctacg ccggcagcag catcttcgtg |
| | | | | atcttcggcg gagggaccaa ggtgaccgtc cta |
| 4 | Light Chain | Human | PROTEIN | Gln Ser Ala Leu Thr Gln Pro Ala Ser Val |
| | Variable Region | VL | | Ser Gly Ser Pro Gly Gln Ser Ile Thr Ile |
| | (VL) of | | | Ser Cys Thr Gly Thr Ser Ser Asp Val Gly |
| | seribantumab | | | Ser Tyr Asn Val Val Ser Trp Tyr Gln Gln |
| | | | | His Pro Gly Lys Ala Pro Lys Leu Ile Ile |
| | | | | Tyr Glu Val Ser Gln Arg Pro Ser Gly Val |
| | | | | Ser Asn Arg Phe Ser Gly Ser Lys Ser Gly |
| | | | | Asn Thr Ala Ser Leu Thr Ile Ser Gly Leu |
| | | | | Gln Thr Glu Asp Glu Ala Asp Tyr Tyr Cys |
| | | | | Cys Ser Tyr Ala Gly Ser Ser Ile Phe Val |
| | | | | Ile Phe Gly Gly Gly Thr Lys Val Thr Val |
| | | | | Leu |
| 5 | Heavy Chain | Human | PROTEIN | His Tyr Val Met Ala |
| | CDR1 (CDRH1) | CDRH1 | | |
| | of seribantumab | | | |
| 6 | Heavy Chain | Human | PROTEIN | Ser Ile Ser Ser Ser Gly Gly Trp Thr Leu |
| | CDR2 (CDRH2) | CDRH2 | | Tyr Ala Asp Ser Val Lys Gly |
| | of seribantumab | | | |
| 7 | Heavy Chain | Human | PROTEIN | Gly Leu Lys Met Ala Thr Ile Phe Asp Tyr |
| | CDR3 (CDRH3) | CDRH3 | | |
| | of seribantumab | | | |
| 8 | Light Chain | Human | PROTEIN | Thr Gly Thr Ser Ser Asp Val Gly Ser Tyr |
| | CDR1 (CDRL1) | CDRL1 | | Asn Val Val Ser |
| | of seribantumab | | | |
| 9 | Light Chain | Human | PROTEIN | Glu Val Ser Gln Arg Pro Ser |
| | CDR2 (CDRL2) | CDRL2 | | |
| | of seribantumab | | | |
| 10 | Light Chain | Human | PROTEIN | Cys Ser Tyr Ala Gly Ser Ser Ile Phe Val |
| | CDR3 (CDRL3) | CDRL3 | | Ile |
| | of seribantumab | | | |
| 11 | Human ERBB3 | Human | PROTEIN | Ser Glu Val Gly Asn Ser Gln Ala Val Cys |
| | | | | Pro Gly Thr Leu Asn Gly Leu Ser Val Thr |
| | | | | Gly Asp Ala Glu Asn Gln Tyr Gln Thr Leu |
| | | | | Tyr Lys Leu Tyr Glu Arg Cys Glu Val Val |
| | | | | Met Gly Asn Leu Glu Ile Val Leu Thr Gly |
| | | | | His Asn Ala Asp Leu Ser Phe Leu Gln Trp |
| | | | | Ile Arg Glu Val Thr Gly Tyr Val Leu Val |
| | | | | Ala Met Asn Glu Phe Ser Thr Leu Pro Leu |
| | | | | Pro Asn Leu Arg Val Val Arg Gly Thr Gln |
| | | | | Val Tyr Asp Gly Lys Phe Ala Ile Phe Val |
| | | | | Met Leu Asn Tyr Asn Thr Asn Ser Ser His |
| | | | | Ala Leu Arg Gln Leu Arg Leu Thr Gln Leu |
| | | | | Thr Glu Ile Leu Ser Gly Gly Val Tyr Ile |
| | | | | Glu Lys Asn Asp Lys Leu Cys His Met Asp |
| | | | | Thr Ile Asp Trp Arg Asp Ile Val Arg Asp |
| | | | | Arg Asp Ala Glu Ile Val Val Lys Asp Asn |
| | | | | Gly Arg Ser Cys Pro Pro Cys His Glu Val |
| | | | | Cys Lys Gly Arg Cys Trp Gly Pro Gly Ser |
| | | | | Glu Asp Cys Gln Thr Leu Thr Lys Thr Ile |
| | | | | Cys Ala Pro Gln Cys Asn Gly His Cys Phe |
| | | | | Gly Pro Asn Pro Asn Gln Cys Cys His Asp |
| | | | | Glu Cys Ala Gly Gly Cys Ser Gly Pro Gln |
| | | | | Asp Thr Asp Cys Phe Ala Cys Arg His Phe |
| | | | | Asn Asp Ser Gly Ala Cys Val Pro Arg Cys |
| | | | | Pro Gln Pro Leu Val Tyr Asn Lys Leu Thr |
| | | | | Phe Gln Leu Glu Pro Asn Pro His Thr Lys |
| | | | | Tyr Gln Tyr Gly Gly Val Cys Val Ala Ser |
| | | | | Cys Pro His Asn Phe Val Val Asp Gln Thr |
| | | | | Ser Cys Val Arg Ala Cys Pro Pro Asp Lys |
| | | | | Met Glu Val Asp Lys Asn Gly Leu Lys Met |
| | | | | Cys Glu Pro Cys Gly Gly Leu Cys Pro Lys |
| | | | | Ala Cys Glu Gly Thr Gly Ser Gly Ser Arg |
| | | | | Phe Gln Thr Val Asp Ser Ser Asn Ile Asp |
| | | | | Gly Phe Val Asn Cys Thr Lys Ile Leu Gly |
| | | | | Asn Leu Asp Phe Leu Ile Thr Gln Gly Asp |
| | | | | Pro Trp His Lys Ile Pro Ala Leu Asp Pro |
| | | | | Glu Lys Leu Asn Val Phe Arg Thr Val Arg |
| | | | | Glu Ile Thr Gly Tyr Leu Asn Ile Gln Ser |
| | | | | Trp Pro Pro His Met His Asn Phe Ser Val |
| | | | | Phe Ser Asn Leu Thr Thr Ile Gly Gly Arg |
| | | | | Ser Leu Tyr Asn Arg Gly Phe Ser Leu Leu |
| | | | | Ile Met Lys Asn Leu Asn Val Thr Ser Leu |
| | | | | Gly Phe Arg Ser Leu Lys Glu Ile Ser Ala |
| | | | | Gly Arg Ile Tyr Ile Ser Ala Asn Arg Gln |
| | | | | Leu Cys Tyr His His Ser Leu Asn Trp Thr |
| | | | | Lys Val Leu Arg Gly Pro Thr Glu Glu Arg |
| | | | | Leu Asp Ile Lys His Asn Arg Pro Arg Arg |
| | | | | Asp Cys Val Ala Glu Gly Lys Val Cys Asp |
| | | | | Pro Leu Cys Ser Ser Gly Gly Cys Trp Gly |
| | | | | Pro Gly Pro Gly Gln Cys Leu Ser Cys Arg |
| | | | | Asn Tyr Ser Arg Gly Gly Val Cys Val Thr |
| | | | | His Cys Asn Phe Leu Asn Gly Glu Pro Arg |
| | | | | Glu Phe Ala His Glu Ala Glu Cys Phe Ser |
| | | | | Cys His Pro Glu Cys Gln Pro Met Glu Gly |
| | | | | Thr Ala Thr Cys Asn Gly Ser Gly Ser Asp |
| | | | | Thr Cys Ala Gln Cys Ala His Phe Arg Asp |
| | | | | Gly Pro His Cys Val Ser Ser Cys Pro His |
| | | | | Gly Val Leu Gly Ala Lys Gly Pro Ile Tyr |
| | | | | Lys Tyr Pro Asp Val Gln Asn Glu Cys Arg |
| | | | | Pro Cys His Glu Asn Cys Thr Gln Gly Cys |
| | | | | Lys Gly Pro Glu Leu Gln Asp Cys Leu Gly |
| | | | | Gln Thr Leu Val Leu Ile Gly Lys Thr His |
| | | | | Leu Thr Met Ala Leu Thr Val Ile Ala Gly |
| | | | | Leu Val Val Ile Phe Met Met Leu Gly Gly |
| | | | | Thr Phe Leu Tyr Trp Arg Gly Arg Arg Ile |
| | | | | Gln Asn Lys Arg Ala Met Arg Arg Tyr Leu |
| | | | | Glu Arg Gly Glu Ser Ile Glu Pro Leu Asp |
| | | | | Pro Ser Glu Lys Ala Asn Lys Val Leu Ala |
| | | | | Arg Ile Phe Lys Glu Thr Glu Leu Arg Ser |
| | | | | Leu Lys Val Leu Gly Ser Gly Val Phe Gly |
| | | | | Thr Val His Lys Gly Val Trp Ile Pro Glu |
| | | | | Gly Glu Ser Ile Lys Ile Pro Val Cys Ile |
| | | | | Lys Val Ile Glu Asp Lys Ser Gly Arg Gln |
| | | | | Ser Phe Gln Ala Val Thr Asp His Met Leu |
| | | | | Ala Ile Gly Ser Leu Asp His Ala His Ile |
| | | | | Val Arg Leu Leu Gly Leu Cys Pro Gly Ser |
| | | | | Ser Leu Gln Leu Val Thr Gln Tyr Leu Pro |
| | | | | Leu Gly Ser Leu Leu Asp His Val Arg Gln |
| | | | | His Arg Gly Ala Leu Gly Pro Gln Leu Leu |
| | | | | Leu Asn Trp Gly Val Gln Ile Ala Lys Gly |
| | | | | Met Tyr Tyr Leu Glu Glu His Gly Met Val |
| | | | | His Arg Asn Leu Ala Ala Arg Asn Val Leu |
| | | | | Leu Lys Ser Pro Ser Gln Val Gln Val Ala |
| | | | | Asp Phe Gly Val Ala Asp Leu Leu Pro Pro |
| | | | | Asp Asp Lys Gln Leu Leu Tyr Ser Glu Ala |
| | | | | Lys Thr |
| 12 | Heavy Chain of | human | PROTEIN | Gln Pro Gln Ile Cys Thr Ile Asp Val Tyr |
| | seribantumab | heavy | | Met Val Met Val Lys Cys Trp Met Ile Asp |
| | | chain | | Glu Asn Ile Arg Pro Thr Phe Lys Glu Leu |
| | | | | Ala Asn Glu Phe Thr Arg Met Ala Arg Asp |
| | | | | Pro Pro Arg Tyr Leu Val Ile Lys Arg Glu |
| | | | | Ser Gly Pro Gly Ile Ala Pro Gly Pro Glu |
| | | | | Pro His Gly Leu Thr Asn Lys Lys Leu Glu |
| | | | | Glu Val Glu Leu Glu Pro Glu Leu Asp Leu |
| | | | | Asp Leu Asp Leu Glu Ala Glu Glu Asp Asn |
| | | | | Leu Ala Thr Thr Thr Leu Gly Ser Ala Leu |
| | | | | Ser Leu Pro Val Gly Thr Leu Asn Arg Pro |
| | | | | Arg Gly Ser Gln Ser Leu Leu Ser Pro Ser |
| | | | | Ser Gly Tyr Met Pro Met Asn Gln Gly Asn |
| | | | | Leu Gly Glu Ser Cys Gln Glu Ser Ala Val |
| | | | | Ser Gly Ser Ser Glu Arg Cys Pro Arg Pro |
| | | | | Val Ser Leu His Pro Met Pro Arg Gly Cys |
| | | | | Leu Ala Ser Glu Ser Ser Glu Gly His Val |
| | | | | Thr Gly Ser Glu Ala Glu Leu Gln Glu Lys |
| | | | | Val Ser Met Cys Arg Ser Arg Ser Arg Ser |
| | | | | Arg Ser Pro Arg Pro Arg Gly Asp Ser Ala |
| | | | | Tyr His Ser Gln Arg His Ser Leu Leu Thr |
| | | | | Pro Val Thr Pro Leu Ser Pro Pro Gly Leu |
| | | | | Glu Glu Glu Asp Val Asn Gly Tyr Val Met |
| | | | | Pro Asp Thr His Leu Lys Gly Thr Pro Ser |
| | | | | Ser Arg Glu Gly Thr Leu Ser Ser Val Gly |
| | | | | Leu Ser Ser Val Leu Gly Thr Glu Glu Glu |
| | | | | Asp Glu Asp Glu Glu Tyr Glu Tyr Met Asn |
| | | | | Arg Arg Arg Arg His Ser Pro Pro His Pro |
| | | | | Pro Arg Pro Ser Ser Leu Glu Glu Leu Gly |
| | | | | Tyr Glu Tyr Met Asp Val Gly Ser Asp Leu |
| | | | | Ser Ala Ser Leu Gly Ser Thr Gln Ser Cys |
| | | | | Pro Leu His Pro Val Pro Ile Met Pro Thr |
| | | | | Ala Gly Thr Thr Pro Asp Glu Asp Tyr Glu |
| | | | | Tyr Met Asn Arg Gln Arg Asp Gly Gly Gly |
| | | | | Pro Gly Gly Asp Tyr Ala Ala Met Gly Ala |
| | | | | Cys Pro Ala Ser Glu Gln Gly Tyr Glu Glu |
| | | | | Met Arg Ala Phe Gln Gly Pro Gly His Gln |
| | | | | Ala Pro His Val His Tyr Ala Arg Leu Lys |
| | | | | Thr Leu Arg Ser Leu Glu Ala Thr Asp Ser |
| | | | | Ala Phe Asp Asn Pro Asp Tyr Trp His Ser |
| | | | | Arg Leu Phe Pro Lys Ala Asn Ala Gln Arg |
| | | | | Thr |
| 13 | Light Chain of | light | PROTEIN | 1 EVQLLESGGG LVQPGGSLRL SCAASGFTFS |
| | seribantumab | heavy | | HYVMAWVRQA PGKGLEWVSS |
| | | chain | | 51 ISSSGGWTLY ADSVKGRFTI SRDNSKNTLY |
| | | | | LQMNSLRAED TAVYYCTRGL |
| | | | | 101 KMATIFDYWG QGTLVTVSSA STKGPSVFPL |
| | | | | APCSRSTSES TAALGCLVKD |
| | | | | 151 YFPEPVTVSW NSGALTSGVH TFPAVLQSSG |
| | | | | LYSLSSVVTV PSSNFGTQTY |
| | | | | 201 TCNVDHKPSN TKVDKTVERK CCVECPPCPA |
| | | | | PPVAGPSVFL FPPKPKDTLM |
| | | | | 251 ISRTPEVTCV VVDVSHEDPE VQFNWYVDGV |
| | | | | EVHNAKTKPR EEQFNSTFRV |
| | | | | 301 VSVLTVVHQD WLNGKEYKCK VSNKGLPAPI |
| | | | | EKTISKTKGQ PREPQVYTLP |
| | | | | 351 PSREEMTKNQ VSLTCLVKGF YPSDIAVEWE |
| | | | | SNGQPENNYK TTPPMLDSDG |
| | | | | 401 SFFLYSKLTV DKSRWQQGNV FSCSVMHEAL |
| | | | | HNHYTQKSLS LSPGK |
| | | | | 1 QSALTQPASV SGSPGQSITI SCTGTSSDVG |
| | | | | SYNVVSWYQQ HPGKAPKLII |
| | | | | 51 YEVSQRPSGV SNRFSGSKSG NTASLTISGL |
| | | | | QTEDEADYYC CSYAGSSIFV |
| | | | | 101 IFGGGTKVTV LGQPKAAPSV TLFPPSSEEL |
| | | | | QANKATLVCL VSDFYPGAVT |
| | | | | 151 VAWKADGSPV KVGVETTKPS KQSNNKYAAS |
| | | | | SYLSLTPEQW KSHRSYSCRV |
| | | | | 201 THEGSTVEKT VAPAECS |

1. A method of treating a subject having a tumor that comprises an NRG1 fusion gene, comprising administering to the subject a therapeutically effective amount of an ERBB3 (HER3) antibody, wherein the antibody is administered at a once weekly dose of 3,000 mg, and wherein the antibody comprises heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively.

2. The method of claim 1, wherein once weekly administration of the antibody is discontinued if it is insufficient to effect treatment.

3. The method of 2, wherein once weekly administration of the antibody is determined to be insufficient to effect treatment if there is clinical disease progression, increased symptoms, tolerance, and/or no clinical improvement, compared to baseline.

4. The method of claim 3, wherein the determination is assessed by radiographic assessment.

5. The method of claim 3, wherein the determination is assessed by “Response Evaluation Criteria in Solid Tumors” (RECIST) version 1.1 guidelines.

6. The method of claim 3, wherein the determination is assessed by one or more tumor markers.

7. The method of claim 6, wherein the one or more tumor markers is selected from the group consisting of carbohydrate antigen (CA19-9), cancer embryonic antigen (CEA), cancer antigen 125 (CA-125), and Cancer Antigen 15-3 (CA 15-3).

8. The method of claim 3, wherein the determination is assessed by liver function tests (LFTs).

9. The method of claim 1, wherein the once weekly antibody dose is reduced by 25% upon resuming treatment after the subject experiences a clinically significant adverse event.

10. The method of claim 9, wherein the once weekly antibody dose is reduced to 2,250 mg.

11. The method of claim 1, wherein the once weekly antibody dose is reduced by 50% upon resuming treatment after the subject experiences two or more clinically significant adverse events.

12. The method of claim 11, wherein the once weekly antibody dose is reduced to 1,500 mg.

13. The method of claim 1, wherein the antibody is administered intravenously.

14. The method of claim 13, wherein the antibody is administered intravenously over about one hour.

15. The method of claim 1, wherein the treatment produces at least one therapeutic effect chosen from a reduction in size of a tumor, reduction in number of metastatic lesions over time, complete response, partial response, and stable disease.

16. The method of claim 1, wherein the subject has been determined to have a tumor comprising an NRG1 fusion gene as measured by a tumor biopsy or liquid biopsy assay.

17. The method of claim 16, wherein the assay includes the polymerase chain reaction (PCR), fluorescence in situ hybridization (FISH), or next-generation sequencing (NGS).

18. The method of claim 17, wherein the NGS is RNA-based or DNA-based testing.

19. The method of claim 1, wherein the tumor is a locally advanced or metastatic solid tumor.

20. The method of claim 1, wherein the tumor is selected from the group consisting of squamous cell carcinoma, lung cancer (e.g., invasive mucinous adenocarcinoma (IMA), small-cell lung cancer, non-small cell lung cancer, squamous non-small cell lung cancer (NSCLC), non-squamous NSCLC), glioma, gastrointestinal cancer, ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, pancreatic ductal adenocarcinoma (PDAC), glioblastoma (glioblastoma multiforme), cervical cancer, stomach cancer, bladder cancer, gallbladder cancer (GBC), bile duct cancer (cholangiocarcinoma), hepatoma, breast cancer, colon carcinoma, and head and neck cancer (or carcinoma), diffuse large B-cell lymphoma (DLBCL), neuroendocrine tumor of the nasopharynx, gastric cancer, germ cell tumor, sarcoma, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, gastroesophageal junction (GEJ) cancer, Kaposi's sarcoma, epidermoid cancer, squamous cell cancer, T-cell lymphoma, environmentally-induced cancer, including those induced by asbestos, and a virus-related cancer or cancer of viral origin (e.g., human papilloma virus (HPV-related or -originating tumors)).

21. The method of claim 20, wherein the tumor type is invasive mucinous adenocarcinoma (IMA).

22. The method of claim 20, wherein the tumor type is ovarian cancer.

23. The method of claim 1, wherein the NRG1 fusion comprises a gene selected from the group consisting of: DOC4, CLU, STMN2, PCM1, CD74; SLC3A2; SDC4; ATP1B1; ROCK1; FOXA1; AKAP13; THBS1; PDE7A; THAP7; SMAD4; RAB3IL1; PMEPA1; STMN2; SLC3A2; VAMP2; RBPMS; WRN; RAB2IL1; SARAF; APP; KIF13B; ADAMS; CDH1; COX10-AS1; DIP2B; DPYSL2; GDF15; HMBOX1; MDK; MRPL13; NOTCH2; PARP8; POMK; SETD4; TNC; TSHZ2; VTCN1; WHSC1L1; INTS9; and ZMYM2.

24. The method of claim 1, wherein the antibody comprises heavy and light chain variable region amino acid sequences comprising SEQ ID NOs: 2 and 4, respectively.

25. The method of claim 1, wherein the antibody comprises heavy chain and light chain amino acid sequences comprising SEQ ID NOs: 12 and 13, respectively.

26. The method of claim 1, further comprising administration of a targeted therapy, radiation, chemotherapy, immunotherapy, and/or chemoimmunotherapy.

27. The method of claim 26, wherein the targeted therapy is against EGFR, ALK, ROS, NTRK, mTOR, PI3K, MEK, ERK, or MET.

28. The method of claim 1, further comprising administration of an anti-estrogen.

29. A kit for treating cancer in a subject having a tumor that comprises an NRG1 fusion gene, the kit comprising:
(a) a dose of an anti-ERBB3 antibody comprising heavy chain CDR1, CDR2, and CDR3 sequences comprising SEQ ID NOs: 5, 6, and 7, respectively, and light chain CDR1, CDR2, and CDR3 sequences comprising SEQ ID NOs: 8, 9, and 10, respectively; and
(b) instructions for using the antibody in the method of claim 1.
